SEQUENCE LISTING (451 sequences)

SEQ ID NO: 1 - protein, 500 aa, Artificial Sequence
  Synthetic
MRGELWLLVLVLREAARALSPQPGAGHDEGPGSGWAAKGTVRGWNRRARESPGHVSEPDRTQLSQDLGGGTLAMDTLPDNRTRVVEDNHSYYVSRLYGPSEPHSRELWVDVAEANRSQVKIHTILSNTHRQASRVVLSFDFPFYGHPLRQITIATGGFIFMGDVIHRMLTATQYVAPLMANFNPGYSDNSTVVYFDNGTVFVVQWDHVYLQGWEDKGSFTFQAALHHDGRIVFAYKEIPMSVPEISSSQHPVKTGLSDAFMILNPSPDVPESRRRSIFEYHRIELDPSKVTSMSAVEFTPLPTCLQHRSCDACMSSDLTFNCSWCHVLQRCSSGFDRYRQEWMDYGCAQEAEGRMCEDFQDEDHDSASPDTSFSPYDGDLTTTSSSLFIDSLTTEDDTKLNPYAGGDGLQNNLSPKTKGTPVHLGTIVGIVLAVLLVAAIILAGIYINGHPTSNAALFFIERRPHHWPAMKFRSHPDHSTYAEVEPSGHEKEGFMEAEQC

SEQ ID NO: 2 - protein, 529 aa, Artificial Sequence
  Synthetic
MARFPKADLAAAGVMLLCHFFTDQFQFADGKPGDQILDWQYGVTQAFPHTEEEVEVDSHAYSHRWKRNLDFLKAVDTNRASVGQDSPEPRSFTDLLLDDGQDNNTQIEEDTDHNYYISRIYGPSDSASRDLWVNIDQMEKDKVKIHGILSNTHRQAARVNLSFDFPFYGHFLREITVATGGFIYTGEVVHRMLTATQYIAPLMANFDPSVSRNSTVRYFDNGTALVVQWDHVHLQDNYNLGSFTFQATLLMDGRIIFGYKEIPVLVTQISSTNHPVKVGLSDAFVVVHRIQQIPNVRRRTIYEYHRVELQMSKITNISAVEMTPLPTCLQFNRCGPCVSSQIGFNCSWCSKLQRCSSGFDRHRQDWVDSGCPEESKEKMCENTEPVETSSRTTTTVGATTTQFRVLTTTRRAVTSQFPTSLPTEDDTKIALHLKDNGASTDDSAAEKKGGTLHAGLIIGILILVLIVATAILVTVYMYHHPTSAASIFFIERRPSRWPAMKFRRGSGHPAYAEVEPVGEKEGFIVSEQC

SEQ ID NO: 3 - protein, 480 aa, Artificial Sequence
  Synthetic
MARFPKADLAAAGVMLLCHFFTDQFQFADGKPGDQILDWQYGVTQAFPHTEEEVEVDSHAYSHRWKRNLDFLKAVDTNRASVGQDSPEPRSFTDLLLDDGQDNNTQIERVNLSFDFPFYGHFLREITVATGGFIYTGEVVHRMLTATQYIAPLMANFDPSVSRNSTVRYFDNGTALVVQWDHVHLQDNYNLGSFTFQATLLMDGRIIFGYKEIPVLVTQISSTNHPVKVGLSDAFVVVHRIQQIPNVRRRTIYEYHRVELQMSKITNISAVEMTPLPTCLQFNRCGPCVSSQIGFNCSWCSKLQRCSSGFDRHRQDWVDSGCPEESKEKMCENTEPVETSSRTTTTVGATTTQFRVLTTTRRAVTSQFPTSLPTEDDTKIALHLKDNGASTDDSAAEKKGGTLHAGLIIGILILVLIVATAILVTVYMYHHPTSAASIFFIERRPSRWPAMKFRRGSGHPAYAEVEPVGEKEGFIVSEQC

SEQ ID NO: 4 - protein, 118 aa, Artificial Sequence
  Synthetic
EVQLLESGGGLVQPGGSLRLSCAASGFTFTSYAMSWVRQAPGKGLEWVSAISAGGGYTYYADSVKGRFTISRDNSKNTLYLQMNSLRAEDTAVYYCARYDYNSYFDSWGQGTLVTVSS

SEQ ID NO: 5 - protein, 109 aa, Artificial Sequence
  Synthetic
QSTQPPSVSGAPGQRVTISCTGSSSNIGAGYDVHWYQQLPGTAPKLLIYGNNNRPSGVPDRFSGSKSGTSASLAITGLQAEDEADYYCQSYDRSLSGWVFGGGTKLTVL

SEQ ID NO: 6 - protein, 121 aa, Artificial Sequence
  Synthetic
QVQLVESGGGVVQPGRSLRLSCAASGYAFSSYGMHWVRQAPGKGLEWVAVISHSGSNKYYANSVKGRFTISRDNSKNTLYLQMNSLRAEDTAVYYCARGLAGWEVYPFDVWGQGTLVTVSS

SEQ ID NO: 7 - protein, 109 aa, Artificial Sequence
  Synthetic
QSTQPPSVSGAPGQRVTISCTGSSSNIGAGFDVHWYQQLPGTAPKLLIYDNTNRPSGVPDRFSGSKSGTSASLAITGLQAEDEADYYCQSYDYSLRVWVFGGGTKLTVL

SEQ ID NO: 8 - protein, 124 aa, Artificial Sequence
  Synthetic
EVQLLESGGGLVQPGGSLRLSCAASGFTFSNYAMSWVRQAPGKGLEWVSTISGGGTYYADSVKGRFTISRDNSKNTLYLQMNSLRAEDTAVYYCARSRYWYTKLISYTYGMDVWGQGTLVTVSS

SEQ ID NO: 9 - protein, 111 aa, Artificial Sequence
  Synthetic
QSVLTQPPSVSGAPGQRVTISCTGSSSNIGATYDVHWYQQLPGTAPKLLIYVNNNRPSGVPDRFSGSKSGTSASLAITGLQAEDEADYYCQSYDSSLRAWVFGGGTKLTVL

SEQ ID NO: 10 - protein, 118 aa, Artificial Sequence
  Synthetic
QVQLVESGGGVVQPGRSLRLSCAASGFTFSNYGMHWVRQAPGKGLEWVAVISYHGRNEYYADSVKGRFTISRDNSKNTLYLQMNSLRAEDTAVYYCARGDDQDAFDPWGQGTLVTVSS

SEQ ID NO: 11 - protein, 107 aa, Artificial Sequence
  Synthetic
DIQMTQSPSSLSASVGDRVTITCRASQSISNYLNWYQQKPGKAPKLLIYAASSLQSGVPSRFSGSGSGTDFTLTISSLQPEDFATYYCQQSFSAPYTFGQGTKVEIK

SEQ ID NO: 12 - protein, 127 aa, Artificial Sequence
  Synthetic
QVQLVESGGGVVQPGRSLRLSCAASGFIFSDYDMHWVRQAPGKGLEWVAVISHSGSNKYYADSVKGRFTISRDNSKNTLYLQMNSLRAEDTAVYYCARDRSVYLYYGYHYYEGFDVWGQGTLVTVSS

SEQ ID NO: 13 - protein, 107 aa, Artificial Sequence
  Synthetic
EIVMTQSPATLSVSPGERATLSCRASQSVYNNLAWYQQKPGQAPRLLIYGASTRATGIPARFSGSGSGTEFTLTISSLQSEDFAVYYCQQYNDLPLTFGQGTKVEIK

SEQ ID NO: 14 - protein, 116 aa, Artificial Sequence
  Synthetic
EVQLLESGGGLVQPGGSLRLSCAASGFTFSDYAMSWVRQAPGKGLEWVSSIYTSGGRTYYADSVKGRFTISRDNSKNTLYLQMNSLRAEDTAVYYCARGYRYFDHWGQGTLVTVSS

SEQ ID NO: 15 - protein, 107 aa, Artificial Sequence
  Synthetic
EIVMTQSPATLSVSPGERATLSCRASQSVRNNLAWYQQKPGQAPRLLIYGASTRATGIPARFSGSGSGTEFTLTISSLQSEDFAVYYCQQYYTWPRTFGQGTKVEIK

SEQ ID NO: 16 - protein, 120 aa, Artificial Sequence
  Synthetic
EVQLLESGGGLVQPGGSLRLSCAASGFTFPNYAMSWVRQAPGKGLEWVSTIYGRGERTYYADSVKGRFTISRDNSKNTLYLQMNSLRAEDTAVYYCARVSVVWSYAFDYWGQGTLVTVSS

SEQ ID NO: 17 - protein, 107 aa, Artificial Sequence
  Synthetic
DIQMTQSPSSLSASVGDRVTITCRASQDISTYLNWYQQKPGKAPKLLIYAASSLQSGVPSRFSGSGSGTDFTLTISSLQPEDFATYYCQQSYSYPLTFGQGTKVEIK

SEQ ID NO: 18 - protein, 119 aa, Artificial Sequence
  Synthetic
QLQLQESGPGLVKPSETLSLTCTVSGGSISSSNYYWGWIRQPPGKGLEWIGSVYYTGRTYYNPSLKSRVTISVDTSKNQFSLRLSSVTAADTAVYYCARVFPYGAVDVWGQGTLVTVSS

SEQ ID NO: 19 - protein, 109 aa, Artificial Sequence
  Synthetic
EIVLTQSPGTLSLSPGERATLSCRASQSVSGSYLAWYQQKPGQAPRLLIYGASSRATGIPDRFSGSGSGTDFTLTISRLEPEDFAVYYCQQYGGSLPYTFGQGTKVEIK

SEQ ID NO: 20 - protein, 118 aa, Artificial Sequence
  Synthetic
QVQLVESGGGVVQPGRSLRLSCAASGFTFSDYGMHWVRQAPGKGLEWVAVISYEGSNEYYADSVKGRFTISRDNSKNTLYLQMNSLRAEDTAVYYCARGSEGSGFDVWGQGTLVTVSS

SEQ ID NO: 21 - protein, 107 aa, Artificial Sequence
  Synthetic
DIQMTQSPSSLSASVGDRVTITCRASQRISNYLNWYQQKPGKAPKLLIYAASSLQSGVPSRFSGSGSGTDFTLTISSLQPEDFATYYCQQSYSPPYTFGQGTKVEIK

SEQ ID NO: 22 - protein, 120 aa, Artificial Sequence
  Synthetic
EVQLLESGGGLVQPGGSLRLSCAASGFTFSTYAMSWVRQAPGKGLEWVSSIYGSGGKTYYADSVKGRFTISRDNSKNTLYLQMNSLRAEDTAVYYCARTDYSLYYQFDYWGQGTLVTVSS

SEQ ID NO: 23 - protein, 107 aa, Artificial Sequence
  Synthetic
DIQMTQSPSSLSASVGDRVTITCRASQSISRYLNWYQQKPGKAPKLLIYAASSLQSGVPSRFSGSGSGTDFTLTISSLQPEDLATYYCQQSYSYPVTFGQGTKVEIK

SEQ ID NO: 24 - protein, 118 aa, Artificial Sequence
  Synthetic
QVQLVESGGGVVQPGRSLRLSCAASGFTFTNYGMHWVRQAPGKGLEWVAVISFDGSNKYYANSVKGRFTISRDNSKNTLYLQMNSLRAEDTAVYYCARGGDEDGFDVWGQGTLVTVSS

SEQ ID NO: 25 - protein, 107 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYRPPYTFGQGTKVEIK

SEQ ID NO: 26 - protein, 129 aa, Artificial Sequence
  Synthetic
EVQLLESGGGLVQPGGSLRLSCAASGFTFSNYAMSWVRQAPGKGLEWVSSITGSGEYTNYADSVKGRFTISRDNSKNTLYLQMNSLRAEDTAVYYCAREVADYWDGGVYYYYDGGFDVWGQGTLVTVSS

SEQ ID NO: 27 - protein, 109 aa, Artificial Sequence
  Synthetic
EIVLTQSPGTLSLSPGERATLSCRASQSVSSNYLAWYQQKPGQAPRLLIYGASSRATGIPDRFSGSGSGTDFTLTISRLEPEDFAVYYCQQYGRSAPITFGQGTKVEIK

SEQ ID NO: 28 - protein, 120 aa, Artificial Sequence
  Synthetic
EVQLLESGGGLVQPGGSLRLSCAASGFTFSSYAMSWVRQAPGKGLEWVSTISGTGGRTYYADSVKGRFTISRDNSKNTLYLQMNSLRAEDTAVYYCARVDHYSGYAFDLWGQGTLVTVSS

SEQ ID NO: 29 - protein, 107 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSYPLTFGQGTKVEIK

SEQ ID NO: 30 - protein, 122 aa, Artificial Sequence
  Synthetic
EVQLLESGGGLVQPGGSLRLSCAASGFTFSSYAMSWVRQAPGKGLEWVSTIYGGGSYTYYADSVKGRFTISRDNSKNTLYLQMNSLRAEDTAVYYCARAEVDDYWYLYMDVWGQGTLVTVSS

SEQ ID NO: 31 - protein, 107 aa, Artificial Sequence
  Synthetic
DIQMTQSPSSLSASVGDRVTITCRASQDIGNYLNWYQQKPGKAPKLLIYAASSLQSGVPSRFSGSGSGTDFTLTISSLQPEDFATYYCQQSYTFPLTFGQGTKVEIK

SEQ ID NO: 32 - protein, 120 aa, Artificial Sequence
  Synthetic
EVQLLESGGGLVQPGGSLRLSCAASGFTFSTYAMSWVRQAPGKGLEWVSTIYGSGGRTYYADSVKGRFTISRDNSKNTLYLQMNSLRAEDTAVYYCARTDHYSYYVMDYWGQGTLVTVSS

SEQ ID NO: 33 - protein, 107 aa, Artificial Sequence
  Synthetic
DIQMTQSPSSLSASVGDRVTITCRASQSISKYLNWYQQKPGKAPKLLIYSASSLQSGVPSRFSGSGSGTDFTLTISSLQPEDCATYYCQQSYSFPLTFGQGTKVEIK

SEQ ID NO: 34 - protein, 121 aa, Artificial Sequence
  Synthetic
EVQLLESGGGLVQPGGSLRLSCAASGFTFSDYAMSWVRQAPGKGLEWVSAIYGSGSYTYYADSVKGRFTISRDNSKNTLYLQMNSLRAEDTAVYYCARTAYSYPYVYFDFWGQGTLVTVSS

SEQ ID NO: 35 - protein, 107 aa, Artificial Sequence
  Synthetic
DIQMTQSPSSLSASVGDRVTITCRASQTITSYLNWYQQKPGKAPKLLIYAASSLQSGVPSRFSGSGSGTDFTLTISSLQPEDFATYYCQQSYTYPLTFGQGTKVEIK

SEQ ID NO: 36 - protein, 126 aa, Artificial Sequence
  Synthetic
EVQLLESGGGLVQPGGSLRLSCAASGFTFGNYAMSWVRQAPGKGLEWVSVISGGGTNTYYADSVKGRFTISRDNSKNTLYLQMNSLRAEDTAVYYCARSDSYYGVYDGYYYGMDVWGQGTLVTVSS

SEQ ID NO: 37 - protein, 107 aa, Artificial Sequence
  Synthetic
DIQMTQSPSSLSASVGDRVTITCRASQTISRYLNWHQQKPGKAPKLLIYAASSLQSGVPSRFSGSGSGTDFTLTISSLQPEDFATYYCQQTYSTPWTFGQGTKVEIK

SEQ ID NO: 38 - protein, 119 aa, Artificial Sequence
  Synthetic
EVQLLESGGGLVQPGGSLRLSCAASGFTFSSYAMSWVRQAPGKGLEWVSTIYASGATTYYADSVKGRFTISRDNSKNTLYLQMNSLRAEDTAVYYCARLKLYHAAFDIWGQGTLVTVSS

SEQ ID NO: 39 - protein, 107 aa, Artificial Sequence
  Synthetic
DIQMTQSPSSLSASVGDRVTITCRASQDISSYLNWYQQKPGKAPKLLIYTASSLQSGVPSRFSGSGSGTDFTLTISSLQPEDFATYYCQQSYSYPLTFGQGTKVEIK

SEQ ID NO: 40 - protein, 120 aa, Artificial Sequence
  Synthetic
EVQLLESGGGLVQPGGSLRLSCAASGFTFRDYAMSWVRQAPGKGLEWVSTIYGSGSRTYYADSVKGRFTISRDNSKNTLYLQMNSLRAEDTAVYYCARVDHVHGYAFDYWGQGTLVTVSS

SEQ ID NO: 41 - protein, 107 aa, Artificial Sequence
  Synthetic
DIQMTQSPSSLSASVGDRVTITCRASQSISKYLNWYQQKPGKAPKLLIYAASSLQSGVPSRFSGSGSGTDFTLTISSLQPEDFATYYCQQSYSFPPTFGQGTKVEIK

SEQ ID NO: 42 - protein, 128 aa, Artificial Sequence
  Synthetic
EVQLLESGGGLVQPGGSLRLSCAASGFTFSNYAMSWVRQAPGKGLEWVSTIYESGGYTYYADSVKGRFTISRDNSKNTLYLQMNSLRAEDTAVYYCARVESYGYGEYITYNYYGFDVWGQGTLVTVSS

SEQ ID NO: 43 - protein, 109 aa, Artificial Sequence
  Synthetic
EIVLTQSPGTLSLSPGERATLSCRASQSVATGYLAWYQQKPGQAPRLLIYGASSRATGIPDRFSGSGSGTDFTLTISRLEPEDFAVYYCQQYESSPPFTFGQGTKVEIK

SEQ ID NO: 44 - protein, 127 aa, Artificial Sequence
  Synthetic
QVQLVQSGAEVKKPGSSVKVSCKASGGTFTSYGIHWVRQAPGQGLEWMGRIVPILGTTNYAQKFQGRVTITADESTSTAYMELSSLRSEDTAVYYCARDPAVTLDTYVYADHGFDVWGQGTLVTVSS

SEQ ID NO: 45 - protein, 107 aa, Artificial Sequence
  Synthetic
EIVMTQSPATLSVSPGERATLSCRASQSVYNNLAWYQQKPGQAPRLLIYGASTRATGIPARFSGSGSGTEFTLTISSLQSEDFAVYYCQQYNDWPLTFGQGTKVEIK

SEQ ID NO: 46 - protein, 126 aa, Artificial Sequence
  Synthetic
EVQLLESGGGLVQPGGSLRLSCAASGFTFKNYAMSWVRQAPGKGLEWVSGISEGGANTYYADSVKGRFTISRDNSKNTLYLQMNSLRAEDTAVYYCAREPPKYGAYSYYGYGFDPWGQGTLVTVSS

SEQ ID NO: 47 - protein, 108 aa, Artificial Sequence
  Synthetic
EIVLTQSPGTLSLSPGERATLSCRASQSVGSNYLAWYQQKPGQAPRLLIYGASSRATGIPDRFSGSGSGTDFTLTISRLEPEDFAVYYCQQYGSSPYTFGQGTKVEIK

SEQ ID NO: 48 - protein, 118 aa, Artificial Sequence
  Synthetic
EVQLLESGGGLVQPGGSLRLSCAASGFTFKGYAMSWVRQAPGKGLEWVSSISVSGAGTYYADSVKGRFTISRDNSKNTLYLQMNSLRAEDTAVYYCARYVRGVSFDVWGQGTLVTVSS

SEQ ID NO: 49 - protein, 109 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYETSPPVTFGQGTKVEIK

SEQ ID NO: 50 - protein, 119 aa, Artificial Sequence
  Synthetic
EVQLLESGGGLVQPGGSLRLSCAASGFTFSNYAMSWVRQAPGKGLEWVSAIGGSGGGTYYADSVKGRFTISRDNSKNTLYLQMNSLRAEDTAVYYCARYGWEYQAFDYWGQGTLVTVSS

SEQ ID NO: 51 - protein, 107 aa, Artificial Sequence
  Synthetic
DIQMTQSPSSLSASVGDRVTITCRASQSISRYLNWYQQKPGKAPKLLIYAASSLQSGVPSRFSGSGSGTDFTLTISSLQPEDFATYYCQQSYSTPWTFGQGTKVEIK

SEQ ID NO: 52 - protein, 119 aa, Artificial Sequence
  Synthetic
EVQLLESGGGLVQPGGSLRLSCAASGFTFSNYAMSWVRQAPGKGLEWVSGITAGGGNTYYADSVKGRFTISRDNSKNTLYLQMNSLRAEDTAVYYCARIAGLFDGFDVWGQGTLVTVSS

SEQ ID NO: 53 - protein, 108 aa, Artificial Sequence
  Synthetic
EIVLTQSPGTLSLSPGERATLSCRASQSVRSSYLAWYQQKPGQAPRLLIYGASSRATGIPDRFSGSGSGTDFTLTISRLEPEDFAVYYCQQYASLPLTFGQGTKVEIK

SEQ ID NO: 54 - protein, 119 aa, Artificial Sequence
  Synthetic
EVQLLESGGGLVQPGGSLRLSCAASGFTFSNYAMSWVRQAPGKGLEWVSGISASGGGTYYADSVKGRFTISRDNSKNTLYLQMNSLRAEDTAVYYCARSSRYGDTFDVWGQGTLVTVSS

SEQ ID NO: 55 - protein, 107 aa, Artificial Sequence
  Synthetic
EIVMTQSPATLSVSPGERATLSCRASLSVGSNLAWYQQKPGQAPRLLIYGASTRATGIPARFSGSGSGTEFTLTISSLQSEDFAVYYCQQYNNLPLTFGQGTKVEIK

SEQ ID NO: 56 - protein, 124 aa, Artificial Sequence
  Synthetic
EVQLLESGGGLVQPGGSLRLSCAASGFTFTDYAMSWVRQAPGKGLEWVSGISVSGGYTYYADSVKGRFTISRDNSKNTLYLQMNSLRAEDTAVYYCARGLYAHHTYYYGGFDYWGQGTLVTVSS

SEQ ID NO: 57 - protein, 107 aa, Artificial Sequence
  Synthetic
EIVMTQSPATLSVSPGERATLSCRASQSVGSNLAWYQQKPGQAPRLLIYGASTRATGIPARFSGSGSGTEFTLTISSLQSEDFAVYYCQQYHNWPPTFGQGTKVEIK

SEQ ID NO: 58 - protein, 127 aa, Artificial Sequence
  Synthetic
EVQLLESGGGLVQPGGSLRLSCAASGFTFRSYAMSWVRQAPGKGLEWVSTIYGTGEDTYYADSVKGRFTISRDNSKNTLYLQMNSLRAEDTAVYYCARDYFWSPRGSIYDYDYFDVWGQGTLVTVSS

SEQ ID NO: 59 - protein, 107 aa, Artificial Sequence
  Synthetic
DIQMTQSPSSLSASVGDRVTITCRASQSISRYLNWYQQKPGKAPKLLIYSASSLQSGVPSRFSGSGSGTDFTLTISSLQPEDFATYYCQQYFSAPYTFGQGTKVEIK

SEQ ID NO: 60 - protein, 118 aa, Artificial Sequence; identical to SEQ ID NO: 48
  Synthetic

SEQ ID NO: 61 - protein, 109 aa, Artificial Sequence
  Synthetic
EIVLTQSPGTLSLSPGERATLSCRASQSVSSNYLAWYQQKPGQAPRLLIYGASSRATGIPDRFSGSGSGTDFTLTISRLEPEDFAVYYCQQYGSSPAVTFGQGTKVEIK

SEQ ID NO: 62 - protein, 127 aa, Artificial Sequence
  Synthetic
EVQLLESGGGLVQPGGSLRLSCAASGFTFSDYAMSWVRQAPGKGLEWVSAISSSGGTTYYADSVKGRFTISRDNSKNTLYLQMNSLRAEDTAVYYCARDAGWSYYGLWIYYYYMDVWGQGTLVTVSS

SEQ ID NO: 63 - protein, 108 aa, Artificial Sequence
  Synthetic
EIVLTQSPGTLSLSPGERATLSCRASQSVRSNYLAWYQQKPGQAPRLLIYGASSRATGIPDRFSGSGSGTDFTLTISRLEPEDFAVYYCQQYGASPQTFGQGTKVEIK

SEQ ID NO: 64 - protein, 124 aa, Artificial Sequence
  Synthetic
EVQLLESGGGLVQPGGSLRLSCAASGFTFKDYAMSWVRQAPGKGLEWVSGISGSGGYTYYADSVKGRFTISRDNSKNTLYLQMNSLRAEDTAVYYCARKLGGFDENYTYGMDVWGQGTLVTVSS

SEQ ID NO: 65 - protein, 109 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPEYTFGQGTKVEIK

SEQ ID NO: 66 - protein, 121 aa, Artificial Sequence
  Synthetic
EVQLLESGGGLVQPGGSLRLSCAASGFTFSTYAMSWVRQAPGKGLEWVSGISAGGGARTYYADSVKGRFTISRDNSKNTLYLQMNSLRAEDTAVYYCARVHHFGYYSLDVWGQGTLVTVSS

SEQ ID NO: 67 - protein, 107 aa, Artificial Sequence
  Synthetic
DIQMTQSPSSLSASVGDRVTITCRASQTISNYLNWYQQKPGKAPKLLIYAASSLQSGVPSRFSGSGSGTDFTLTISSLQPEDFATYYCQQSYTYPLTFGQGTKVEIK

SEQ ID NO: 68 - protein, 118 aa, Artificial Sequence
  Synthetic
EVQLLESGGGLVQPGGSLRLSCAASGFTFSSYAMSWVRQAPGKGLEWVSVIYGSGGRTYYADSVKGRFTISRDNSKNTLYLQMNSLRAEDTAVYYCARHDPRYQLDVWGQGTLVTVSS

SEQ ID NO: 69 - protein, 107 aa, Artificial Sequence
  Synthetic
DIQMTQSPSSLSASVGDRVTITCRASQSIGSYLNWYQQKPGKAPKLLIYTASSLQSGVPSRFSGSGSGTDFTLTISSLQPEDFATYYCQQSYTYPLTFGQGTKVEIK

SEQ ID NO: 70 - protein, 118 aa, Artificial Sequence
  Synthetic
QVQLVESGGGVVQPGRSLRLSCAASGFTFSDYGMHWVRQAPGKGLEWVAVISYDGSRKYYANSVKGRFTISRDNSKNTLYLQMNSLRAEDTAVYYCARGSYSDGFDVWGQGTLVTVSS

SEQ ID NO: 71 - protein, 107 aa, Artificial Sequence
  Synthetic
DIQMTQSPSSLSASVGDRVTITCRASQSISRYLNWYQQKTGKAPKLLIYGASSLQSGVPSRFSGSGSGTDFTLTISSLQPEDFATYYCQQSYSTPYTFGQGTKVEIK

SEQ ID NO: 72 - protein, 125 aa, Artificial Sequence
  Synthetic
QVQLVQSGAEVKKPGSSVKVSCKASGGTFSTEYISWVRQAPGQGLEWMGRIIPVLGITNYAQKFQGRVTITADESTSTAYMELSSLRSEDTAVYYCARHLGPPYLVTYSHGFDVWGQGTLVTVSS

SEQ ID NO: 73 - protein, 107 aa, Artificial Sequence
  Synthetic
EIVMTQSPATLSVSPGERATLSCRASQSLGTNLAWYQQKPGQAPRLLIYGASTRATGIPARFSGSGSGTEFTLTISSLQSEDFAVYYCQQYYEAPPTFGQGTKVEIK

SEQ ID NO: 74 - protein, 119 aa, Artificial Sequence
  Synthetic
EVQLLESGGGLVQPGGSLRLSCAASGFTFSNYAMSWVRQAPGKGLEWVSAIYGSGGKTYYADSVKGRFTISRDNSKNTLYLQMNSLRAEDTAVYYCARLKYGFYQFDYWGQGTLVTVSS

SEQ ID NO: 75 - protein, 107 aa, Artificial Sequence
  Synthetic
DIQMTQSPSSLSASVGDRVTITCRASQSIRNYLNWYEQKPGKAPKLLIYAASSLQSGVPSRFSGSGSGTDFTLTISSLQPEDFATYYCQQSYSYPLTFGQGTKVEIK

SEQ ID NO: 76 - protein, 120 aa, Artificial Sequence
  Synthetic
EVQLLESGGGLVQPGGSLKLSCAASGFTFSNYAMSWVRQAPGKGLEWVSVIYAGGARTYYADSVKGRFTISRDNSKNTLYLQMNSLRAEDTAVYYCARWGGDGFYAMDYWGQGTLVTVSS

SEQ ID NO: 77 - protein, 107 aa, Artificial Sequence
  Synthetic
DIQMTQSPSSLSASVGDRVTITCRASQTISTYLNWYQQKPGKAPKLLIYAASILQSGVPSRFSGSGSGTDFTLTISSLQPEDFATYYCQQSYTVPLTFGQGTKVEIK

SEQ ID NO: 78 - protein, 124 aa, Artificial Sequence
  Synthetic
EVQLLESGGGLVQPGGSLRLSCAASGFTFSNYAMSWVRQAPGKGLEWVSAISGSGGTTYYADSVKGRFTISRDNSKNTLYLQMNSLRAEDTAVYYCARIKKYGSAGYTVGFDSWGQGTLVTVSS

SEQ ID NO: 79 - protein, 107 aa, Artificial Sequence
  Synthetic
EIVMTQSPATLSVSPGERATLSCRASQSVGSYLAWYQQKPGQAPRLLIYGASTRATGIPARFSGSGSGTEFTLTISSLQSEDFAVYYCQQYYNAPITFGQGTKVEIK

SEQ ID NO: 80 - protein, 124 aa, Artificial Sequence
  Synthetic
QLQLQESGPGLVKPSETLSLTCTVSGGSISSSSYYWGWIRQPPGKGLEWIGNIYYTGTTYYNPSLKSRVTISVDTSKNQFSLKLSSVTAADTAVYYCARIKVPWYYSSYYFDYWGQGTLVTVSS

SEQ ID NO: 81 - protein, 111 aa, Artificial Sequence
  Synthetic
QSVLTQPPSVSGAPGQRVTISCTGSSSNIGAGYDVHWYQQLPGTAPKLLIYGNKNRPSGVPDRFSGSKSGTSASLAITGLQAEDEADYYCQSYDTSLSAWVFGGGTKLTVL

SEQ ID NO: 82 - protein, 117 aa, Artificial Sequence
  Synthetic
EVQLLESGGGLVQPGGSLRLSCAASGFTFKNYAMSWVRQAPGKGLEWVSAISASGGYTYYADSVKGRFTISRDNSKNTLYLQMNSLRAEDTAVYYCARGRWGAFDYWGQGTLVTVSS

SEQ ID NO: 83 - protein, 111 aa, Artificial Sequence
  Synthetic
QSVLTQPPSVSGAPGQRVTISCTGSSSNIGSGYDVHWYQQLPGTAPKLLIYGNRNRPSGVPDRFSGSKSGTSASLAITGLQAEDEADYYCQSYDSSLKTWVFGGGTKLTVL

SEQ ID NO: 84 - protein, 117 aa, Artificial Sequence
  Synthetic
EVQLLESGGGLVQPGGSLKLSCAASGFTFSDYAMSWVRQAPGKGLEWVSGISRGGARTYYADSVKGRFTISRDNSKNTLYLQMNSLRAEDTAVYYCARYLFGHYMDWGQGTLVTVSS

SEQ ID NO: 85 - protein, 111 aa, Artificial Sequence
  Synthetic
QSVLTQPPSVSGAPGQRVTISCTGSSSNIGAGYDVHWYQQLPGTAPKLLIYGNTNRPSGVPDRFSGSKSGTSASLAITGLQAEDEADYYCQSYDGRLGVSVFGGGTKLTVL

SEQ ID NO: 86 - protein, 118 aa, Artificial Sequence
  Synthetic
QVQLVESGGGVVQPGRSLRLSCAASGFTFSRYAMHWVRQAPGKGLEWVAVISHSGSTKYYANSVKGRFTISRDNSKNTLYLQMNSLRAEDTAVYYCARSRGWRGFDSWGQGTLVTVSS

SEQ ID NO: 87 - protein, 110 aa, Artificial Sequence
  Synthetic
QSVLTQPPSVSGAPGQRVTISCTGSSSNIGAYVVHWYQQLPGTAPKLLIYGNTNRPSGVPDRFSGSKSGTSASLAITGLQAEDEADYYCQSYDRTLSSYVFGGGTKLTVL

SEQ ID NO: 88 - protein, 127 aa, Artificial Sequence
  Synthetic
QVQLVESGGGVVQPGRSLRLSCAASGFAFSGYGMHWVRQAPGKGLEWVAVISHHGSYKYYANSVKGRFTISRDNSKNTLYLQMNSLRAEDTAVYYCARGGADVGYFYSTFYYYMDVWGQGTLVTVSS

SEQ ID NO: 89 - protein, 111 aa, Artificial Sequence
  Synthetic
QSVLTQPPSVSGAPGQRVTISCTGSSSNIGAAFDVHWYQQLPGTAPKLLIYDNYNRPSGVPDRFSGSKSGTSASLAITGLQAEDEADYYCQSYDSSLSVWVFGGGTKLTVL

SEQ ID NO: 90 - protein, 120 aa, Artificial Sequence
  Synthetic
EVQLLESGGGLVQPGGSLRLSCAASGFTFSNYAMSWVRQAPGKGLEWVSAISRGGGYTYYADSVKGRFTISRDNSKNTLYLQMNSLRAEDTAVYYCARWGGDGFYAMDYWGQGTLVTVSS

SEQ ID NO: 91 - protein, 111 aa, Artificial Sequence
  Synthetic
QSVLTQPPSVSGAPGQRVTISCTGSSSNIGAGYDVHWYQQLPGTAPKLLIYANNNRPSGVPDRFSGSKSGTSASLAITGLQAEDEADYYCQSYDYSLSGWVFGGGTKLTVL

SEQ ID NO: 92 - protein, 122 aa, Artificial Sequence
  Synthetic
QLQLQESGPGLVKPSETLSLTCTVSGGSISSRGYYWAWIKQPPGKGLEWIGSIYYSGSTYYNPSLKSRVTISVDTSKNQFSLKLSSVTAADTAVYYCARLVSYGAYYVFDYWGQGTLVTVSS

SEQ ID NO: 93 - protein, 111 aa, Artificial Sequence
  Synthetic
QSVLTQPPSVSGAPGQRVTISCTGSSSNIGAGYDVHWYQQLPGTAPKLLIYGNRNRPSGVPDRFSGSKSGTSASLAITGLQAEDEADYYCQSYDTRLSAWVFGGGTKLTVL

SEQ ID NO: 94 - protein, 115 aa, Artificial Sequence
  Synthetic
EVQLLESGGGLVRPGGSLRLSCAASGFTFSSYAMSWVRQAPGKGLEWVSAISGSGGSTYYADSVKGRFTISRDNSKNTLYLQMNSLRAEDTAVYYCARDKRLTYWGQGTMVTVSS

SEQ ID NO: 95 - protein, 110 aa, Artificial Sequence
  Synthetic
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSARVFGGGTKVTVL

SEQ ID NO: 96 - protein, 118 aa, Artificial Sequence
  Synthetic
QVQLVQSGAEVKKPGSSVKVSCKASGGTFSSYAINWVRQAPGQGLEWMGRIIPLLETADYAQKFQGRVTITADESTSTAYMELSSLRSEDTAVYYCAREWPGEYFDVWGQGTLVTVSS

SEQ ID NO: 97 - protein, 108 aa, Artificial Sequence
  Synthetic
EIVLTQSPGTLSLSPGERATLSCRASQSVASSYLAWYQQKPGQAPRLLIYGASSRATGIPDRFSGSGSGTDFTLTISRLEPEDFAVYYCQQYGSSQLTFGQGTKVEIK

SEQ ID NO: 98 - protein, 118 aa, Artificial Sequence
  Synthetic
QVQLVESGGGVVQPGRSLRLSCAASGFTFRDYGMHWVRQAPGKGLEWVAVISYHGRNEYYANSVKGRFTISRDNSKNTLYLQMNSLRAEDTAVYYCARGAYGDDFDVWGQGTLVTVSS

SEQ ID NO: 99 - protein, 107 aa, Artificial Sequence
  Synthetic
DIQMTQSPSSLSASVGDRVTITCRASQSISSYLNWYQQKTGKAPKLLIYAASSLQSGVPSRFSGSGSGTDFTLTISSLQPEDFATYYCQQSYSAPYTFGQGTKVEIK

SEQ ID NO: 100 - protein, 127 aa, Artificial Sequence
  Synthetic
EVQLLESGGGLVQPGGSLRLSCAASGFTFSNYAMSWVRQAPGKGLEWVSTISGGGRTTNYADSVKGRFTISRDNSKNTLYLQMNSLRAEDTAVYYCARSTPIPSYYPYIYSYYFDVWGQGTLVTVSS

SEQ ID NO: 101 - protein, 107 aa, Artificial Sequence
  Synthetic
EIVMTQSPATLSVSPGERATLSCRASQSVSNNLAWYQQKPGQAPRLLIYGASTRATGIPARFSGSGSGTEFTLTISSLQSEDFAVYYCQQYGNYPPTFGQGTKVEIK

SEQ ID NO: 102 - protein, 121 aa, Artificial Sequence
  Synthetic
EVQLLESGGGLVQPGGSLRLSCAASGFTFSDYAMSWVRQAPGKGLEWVSSISSSGANTYYADSGKGRFTISRDNSKNILYLQMNSLRAEDTAVYYCARSVVTWVTYAFDYWGQGTLVTVSS

SEQ ID NO: 103 - protein, 111 aa, Artificial Sequence
  Synthetic
QSVLTQPPSVSGAPGQRVTISCTGSSSNIGAGYDVHWYQQLPGTAPKLLIYDNTNRPSGVPDRFSGSKSGTSASLAITGLQAEDEADYYCQSYDSGLSGWVFGGGTKLTVL

SEQ ID NO: 104 - protein, 121 aa, Artificial Sequence
  Synthetic
EVQLLESGGGLVQPGGSLRLSCAASGFTFSNYAMSWVRQAPGKGLEWVSAISATGGATYYADSVKGRFTISRDNSKNTLYLQMNSLRAEDTAVYYCARSVRERYTYYMDYWGQGTLVTVSS

SEQ ID NO: 105 - protein, 111 aa, Artificial Sequence
  Synthetic
QSVLTQPPSVSGAPGQRVTISCTGSSSNIGAGYDVHWYQQLPGTAPKLLIYDNTNRPSGVPDRFSGSKSGTSASLAITGLQAEDEADYYCQSYDVSLGVWVFGGGTKLTVL

SEQ ID NO: 106 - protein, 118 aa, Artificial Sequence
  Synthetic
QVQLVQSGAEVKKPGSSVKVSCKASGGTFSNYYISWVRQAPGQGLEWMGGIIPVFGVAHYAQKFQGRVTITADESTSTAYMELSSLRSEDTAVYYCARGYPGEYFDYWGQGTLVTVSS

SEQ ID NO: 107 - protein, 109 aa, Artificial Sequence
  Synthetic
EIVLTQSPGTLSLSPGERATLSCRASQSVSSSYLAWYQQKPGQAPRLLIYAASSRATGIPDRFSGSGSGTDFTLTISRLEPEDFAVYYCQHYGGSPGYTFGQGTKVEIK

SEQ ID NO: 108 - protein, 120 aa, Artificial Sequence
  Synthetic
EVQLLESGGGLVQPGGSLRLSCAASGFTFGSYAMSWVRQAPGKGLEWVSSIYAGGGRTYYADSVKGRFTISRDNSKNTLYLQMNSLRAEDTAVYYCARWGGDGFYAMDYWGQGTLVTVSS

SEQ ID NO: 109 - protein, 107 aa, Artificial Sequence
  Synthetic
DIQMTQSPSSLSASVGDRVTITCRASQTISNYLNWYQQKPGKAPKLLIYGASSLQSGVPSRFSGSGSGTDFTLTISSLQPEDFATYYCQQSFTFPLTFGQGTKVEIK

SEQ ID NO: 110 - protein, 121 aa, Artificial Sequence
  Synthetic
EVQLLESGGGLVQPGGSLRLSCAASGFTFSDYAMSWVRQAPGKGLEWVSTIYSGGVRTYYADSVKGRFTISRDNSKNTLYLQMNSLRAEDTAVYYCARGLVSFRGYAFDYWGQGTLVTVSS

SEQ ID NO: 111 - protein, 107 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYTIPPTFGQGTKVEIK

SEQ ID NO: 112 - protein, 121 aa, Artificial Sequence
  Synthetic
EVQLLESGGGLVQPGGSLRLSCAASGFTFRNYAMSWVRQAPGKGLEWVSSISGGGINTYYADSVKGRFTISRDNSKNTLYLQMNSLRAEDTAVYYCARVRSYSGPGYFDYWGQGTLVTVSS

SEQ ID NO: 113 - protein, 107 aa, Artificial Sequence
  Synthetic
EIVMTQSPATLSVSPGDRATLSCRASRSVSSNLAWYQQKPGQAPRLLIYGASTRATGIPARFSGSGSGTEFTLTISSLQSEDFAVYYCQQYYNWPLTFGQGTKVEIK

SEQ ID NO: 114 - protein, 118 aa, Artificial Sequence
  Synthetic
EVQLLESGGGLVQPGGSLRLSCAASGFTFSNYAMSWVRQAPGKGLEWVSVIYGSGARTYYADSVKGRFTISRDNSKNTLYLQMNSLRAEDTAVYYCARISPHYDFDVWGQGTLVTVSS

SEQ ID NO: 115 - protein, 107 aa, Artificial Sequence
  Synthetic
DIQMTQSPSSLSASVGDRVTITCRASQTISKYLNWYQQKPGKAPKLLIYAASSLQSGVPSRFSGSGSGTDFTLTISSLQPEDFATYYCQQSYSYPITFGQGTKVEIK

SEQ ID NO: 116 - protein, 120 aa, Artificial Sequence
  Synthetic
EVQLLESGGGLVQPGGSLRLSCAASGFTFSNYAMSWVRQAPGKGLEWVSAISGGGSRTYYADSVKGRFTISRDNSKNTLYLQMNSLRAEDTAVYYCAREARVLGYSLDYWGQGTLVTVSS

SEQ ID NO: 117 - protein, 107 aa, Artificial Sequence
  Synthetic
DIQMTQSPSSLSASVGDRVTITCRASQRIGKYLNWYQQKPGKAPKLLIYAASSLQSGVPSRFSGSGSGTDFTLTISSLQPEDFATYYCQQSYSYPLTFGQGTKVEIK

SEQ ID NO: 118 - protein, 119 aa, Artificial Sequence
  Synthetic
EVQLLESGGGLVQPGGSLRLSCAASGFTFSNYAMSWVRQAPGKGLEWVSAIYGSGGRTYYADSVKGRFTISRDNSKNTLYLQMNSLRAEDTAVYYCARLRYGFYQFDYWGQGTLVTVSS

SEQ ID NO: 119 - protein, 107 aa, Artificial Sequence; identical to SEQ ID NO: 75
  Synthetic

SEQ ID NO: 120 - protein, 120 aa, Artificial Sequence
  Synthetic
EVQLLESGGGLVQPGGSLRLSCAASGFTFTDYAMSWVRQAPGKGLEWVSTIYSSGHRTYYADSVKGRFTISRDNSKNTLYLQMNSLRAEDTAVYYCARLYPLHSYAFDHWGQGTLVTVSS

SEQ ID NO: 121 - protein, 107 aa, Artificial Sequence
  Synthetic
DIQMTQSHSSLSASVGDRVTITCRASQSISRYLNWYQQKPGKAPKLLIYAASSLQSGVPSRFSGSGSGTDFTLTISSLQPEDFATYYCQQSYSVPLTFGQGTKVEIK

SEQ ID NO: 122 - protein, 125 aa, Artificial Sequence
  Synthetic
EVQLLESGGGLVQPGGSLRLSCAASGFTFKSYAMSWVRQAPGKGLEWVSLISSSGENTYYADSVKGRFTISRDNSKNTLYLQMNSLRAEDTAVYYCARVRFGYGSWRYRKYMDVWGQGTLVTVSS

SEQ ID NO: 123 - protein, 107 aa, Artificial Sequence
  Synthetic
DIQMTQSPSSLSASVGDRVTITCRASQSIATYLNWYQQKPGKAPKLLIYGASSLQSGVPSRFSGSGSGTDFTLTISSLQPEDFATYYCQQSYSTPLTFGQGTKVEIK

SEQ ID NO: 124 - protein, 120 aa, Artificial Sequence
  Synthetic
EVQLLESGGGLVQPGGSLRLSCAASGFTFSSYAMSWVRQAPGKGLEWVSTISASGATYYADSVKGRFTISRDNSKNTLYLQMNSLRAEDTAVYYCARGPYSYYYYSFDYWGQGTLVTVSS

SEQ ID NO: 125 - protein, 107 aa, Artificial Sequence
  Synthetic
DIQMTQSPSSLSASVGDRVTITCRASQTISTYLNWYQQKPGKAPKLLIYAASSLQSGVPSRFSGSGSGTDFTLTISSLQPEDFATYYCQQSYRYPLTFGQGTKVEIK

SEQ ID NO: 126 - protein, 118 aa, Artificial Sequence
  Synthetic
EVQLLESGGGLVQPGGSLRLSCAASGFTFSSYAMSWVRQAPGKGLEWVSTIYVGGHRTYYADSVKGRFTISRDNSKNTLYLQMNSLRAEDTAVYYCARVGWSWGFDPWGQGTLVTVSS

SEQ ID NO: 127 - protein, 107 aa, Artificial Sequence
  Synthetic
DIQMTQSPSSLSASVGDRVTITCRASQTISKYLNWYQQKPGKAPKLLIYSASSLQSGVPSRFSGSGSGTDFTLTISSLQPEDFATYYCQQSYTYPLTFGQGTKVEIK

SEQ ID NO: 128 - protein, 124 aa, Artificial Sequence
  Synthetic
EVQLVESGGGLVKPGGSLRLSCAASGFTFTNAWMSWVRQAPGKGLEWVGRIKSITEGGTTDYAAPVKGRFTISRDDSKNTLYLQMNSLKTEDTAVYYCARVWHTDDYYEGFDVWGQGTLVTVSS

SEQ ID NO: 129 - protein, 108 aa, Artificial Sequence
  Synthetic
EIVLTQSPGTLSLSPGERATLSCRASQSVSGSYLAWYQQKPGQAPRLLIYDASSRATGIPDRFSGSGSGTDFTLTISRLEPEDFAVYYCQQYGTTPQTFGQGTKVEIK

SEQ ID NO: 130 - protein, 120 aa, Artificial Sequence
  Synthetic
EVQLLESGGGLVQPGGSLRLSCAASGFTFRSYAMSWVRQAPGKGLEWVSTIYGSGARTYYADSVKGRFTISRDNSKNTLYLQMNSLRAEDTAVYYCARWGGDGFYAMDYWGQGTLVTVSS

SEQ ID NO: 131 - protein, 107 aa, Artificial Sequence
  Synthetic
DIQMTQSPSSLSASVGDRVTITCRASQSIYTYLNWYQQKPGKAPKLLIYAASSLQSGVPSRFSGSGSGTDFTLTISSLQPEDFATYYCQQSYSFPLTFGQGTKVEIK

SEQ ID NO: 132 - protein, 118 aa, Artificial Sequence
  Synthetic
EVQLLESGGGLVQPGGSLRLSCAASGFTFSSYAMSWVRQAPGKGLEWVSGISTSGGTTYYADSVKGRFTISRDNSKNTLYLQMNSLRAEDTAVYYCARVVGRAVFDIWGQGTLVTVSS

SEQ ID NO: 133 - protein, 108 aa, Artificial Sequence
  Synthetic
EIVLTQSPGTLSLSPGERATLSCRASQSVSSTYLAWYQQKPGQAPRLLIYAASSRATGIPDRFSGSGSGTDFTLTISRLEPEDFAVYYCQQYAALPITFGQGTKVEIK

SEQ ID NO: 134 - protein, 120 aa, Artificial Sequence
  Synthetic
QVQLVQSGAEVKKPGSSVKVSCKASGGTFSDEAIHWVRQAPGQGLEWMGRIIPVLGIASYAQKFQGRVTITADESTSTAYMELSSLRSEDTAVYYCARGLSRKAFYFDYWGQGTLVTVSS

SEQ ID NO: 135 - protein, 107 aa, Artificial Sequence
  Synthetic
DIQMTQSPSSLSASVGDRVTITCRASQTIGNYLNWYQQKPGKAPKLLIYVASSLQSGVPSRFSGSGSGTDFTLTISSLQPEDFATYYCQQSYRYPYTFGQGTKVEIK

SEQ ID NO: 136 - protein, 126 aa, Artificial Sequence
  Synthetic
EVQLLESGGGLVQPGGSLRLSCAASGFTFSNYAMSWVRQAPGKGLEWVSTIYRGAGTYYADSVKGRFTISRDNSKNTLYLQMNSLRAEDTAVYYCARDASWYGPFSYYYYYGFDVWGQGTLVTVSS

SEQ ID NO: 137 - protein, 107 aa, Artificial Sequence
  Synthetic
EIVMTQSPATLSVSPGERATLSCRASQSVYTNLAWYQQKPGQAPRLLIYDASTRATGIPARFSGSGSGTEFTLTISSLQSEDFAVYYCQQYNDWPLTFGQGTKVEIK

SEQ ID NO: 138 - protein, 124 aa, Artificial Sequence
  Synthetic
EVQLLESGGGLVQPGGSLRLSCAASGFTFSDYAMSWVRQAPGKGLEWVSIITESGVNTYYADSVKGRFTISRDNSKNTLYLQMNSLRAEDTAVYYCARHPSVGSASRSYYFDVWGQGTLVTVSS

SEQ ID NO: 139 - protein, 108 aa, Artificial Sequence
  Synthetic
EIVLTQSPGTLSLSPGERATLSCRASQSVGSSYLAWYQQKPGQAPRLLIYAASSRATGIPDRFSGSGSGTDFTLTISRLEPEDFAVYYCQQYYSAPLTFGQGTKVEIK

SEQ ID NO: 140 - protein, 127 aa, Artificial Sequence
  Synthetic
EVQLLESGGGLVQPGGSLRLSCAASGFTFTDYAMSWVRQAPGKGLEWVSGISGSGGVGTYYADSVKGRFTISRDNSKNTLYLQMNSLRAEDTAVYYCARGEYGSSIAYSYYYGFDVWGQGTLVTVSS

SEQ ID NO: 141 - protein, 107 aa, Artificial Sequence
  Synthetic
EIVMTQSPATLSVSPGERATLSCRASQSVYNNLAWYQQKPGQAPRLLIYDASTRATGIPARFSGSGSGTEFTLTISSLQSEDFAVYYCQQYNDWPYTFGQGTKVEIK

SEQ ID NO: 142 - protein, 120 aa, Artificial Sequence; identical to SEQ ID NO: 116
  Synthetic

SEQ ID NO: 143 - protein, 107 aa, Artificial Sequence; identical to SEQ ID NO: 117
  Synthetic

SEQ ID NO: 144 - protein, 119 aa, Artificial Sequence
  Synthetic
EVQLLESGGGLVQPGGSLRLSCAASGFTFSGYAMSWVRQAPGKGLEWVSAIRGGGGTYYADSVKGRFTISRDNSKNTLYLQMNSLRAEDTAVYYCARVGPLRLYGFDYWGQGTLVTVSS

SEQ ID NO: 145 - protein, 107 aa, Artificial Sequence
  Synthetic
DIQMTQSPSSLSASVGDRVTITCRASQSISNYLNWYQQKPGKAPKLLIYAASSLQSGVPSRFSGSGSGTDFTLTISSLQPEDFATYYCQQSYSYPLTFGQGTKVEIK

SEQ ID NO: 146 - protein, 126 aa, Artificial Sequence
  Synthetic
EVQLLESGGGLVQPGGSLRLSCAASGFTFSSYAMSWVRQAPGKGLEWVSVISGSGGNTYYADSVKGRFTISRDNSKNTLYLQMNSLRAEDTAVYYCAREVRHDSYYYYYYSGMDVWGQGTLVTVSS

SEQ ID NO: 147 - protein, 107 aa, Artificial Sequence
  Synthetic
EIVMTQSPATLSVSPGERATLSCRASQSVYNNLAWYQQKPGQAPRLLIYDASTRATGIPARFSGSGSGTEFTLTISSLQSEDFAVYYCQQYSDYPFTFGQGTKVEIK

SEQ ID NO: 148 - protein, 125 aa, Artificial Sequence
  Synthetic
QVQLVQSGAEVKKPGSSVKVSCKASGGTFSNYAINWVRQAPGQGLEWMGRIIPTLGIANYAQKFQGRVTITADESTSTAYMELSSLRSEDTAVYYCARAAYFAGPAARYGYFDIWGQGTLVTVSS

SEQ ID NO: 149 - protein, 108 aa, Artificial Sequence
  Synthetic
EIVLTQSPGTLSLSPGERATLSCRASQSVRSNYLAWYQQKPGQAPRLLIYDASSRATGIPDRFSGSGSGTDFTLTISRLEPEDFAVYYCQQYAYAPWTFGQGTKVEIK

SEQ ID NO: 150 - protein, 118 aa, Artificial Sequence
  Synthetic
EVQLLESGGGLVQPGGSLRLSCAASGFTFRGYAMSWVRQAPGKGLEWVSSISVSGAGTYYADSVKGRFTISRDNSKNTLYLQMNSLRAEDTAVYYCARYVRGVSFDVWGQGTLVTVSS

SEQ ID NO: 151 - protein, 109 aa, Artificial Sequence; identical to SEQ ID NO: 61
  Synthetic

SEQ ID NO: 152 - protein, 119 aa, Artificial Sequence; identical to SEQ ID NO: 118
  Synthetic

SEQ ID NO: 153 - protein, 107 aa, Artificial Sequence; identical to SEQ ID NO: 75
  Synthetic

SEQ ID NO: 154 - protein, 121 aa, Artificial Sequence; identical to SEQ ID NO: 110
  Synthetic

SEQ ID NO: 155 - protein, 107 aa, Artificial Sequence; identical to SEQ ID NO: 111
  Synthetic

SEQ ID NO: 156 - protein, 118 aa, Artificial Sequence; identical to SEQ ID NO: 70
  Synthetic

SEQ ID NO: 157 - protein, 107 aa, Artificial Sequence
  Synthetic
DIQMTQSPSSLSASVGDRMTITCRASQSISNYLNWYQQKPGKAPKLLIYAASSLQSGVPSRFSGSGSGTDFTLTISSLQPEDFATYYCQQSYAPPYTFGQGTKVEIK

SEQ ID NO: 158 - protein, 119 aa, Artificial Sequence
  Synthetic
EVQLLESGGGLVQPGGSLRLSCAASGFTFSSYAMSWVRQAPGKGLEWVSGISESGGGTYYADSVKGRFTISRDNSKNTLYLQMNSLRAEDTAVYYCARLVPWQLAFDVWGQGTLVTVSS

SEQ ID NO: 159 - protein, 107 aa, Artificial Sequence
  Synthetic
EIVMTQSPATLSVSPGERATLSCRASQSVYNNLAWYQQKPGQAPRLLIYDASTRATGIPARFSGSGSGTEFTLTISSLQSEDFAVYYCQQYYYWPITFGQGTKVEIK

SEQ ID NO: 160 - protein, 126 aa, Artificial Sequence; identical to SEQ ID NO: 146
  Synthetic

SEQ ID NO: 161 - protein, 107 aa, Artificial Sequence; identical to SEQ ID NO: 147
  Synthetic

SEQ ID NO: 162 - protein, 126 aa, Artificial Sequence
  Synthetic
QVQLVQSGAEVKKPGSSVKVSCKASGGTFSSEAISWVRQAPGQGLEWMGRIIPISGRPNYAQKFQGRVTITADESTSTAYMELSSLRSEDTAVYYCAREASYYVAGYYRYYGFDVWGQGTLVTVSS

SEQ ID NO: 163 - protein, 107 aa, Artificial Sequence
  Synthetic
EIVMTQSPATLSVSPGERATLSCRASRSLSNNLAWYQQKPGQAPRLLIYDASTRATGIPARFSGSGSGTEFTLTISSLQSEDFAVYYCQQYNAWPYTFGQGTKVEIK

SEQ ID NO: 164 - protein, 126 aa, Artificial Sequence; identical to SEQ ID NO: 146
  Synthetic

SEQ ID NO: 165 - protein, 107 aa, Artificial Sequence; identical to SEQ ID NO: 147
  Synthetic

SEQ ID NO: 166 - protein, 126 aa, Artificial Sequence
  Synthetic
QVQLVESGGGLVKPGGSLRLSCAASGFTFSNAWMSWVRQAPGKGLEWVGRIKSKIEGGTTDYAPPVKGRFTISRDDSKNTLYLQMNSLKTEDTAVYYCARDPTWYVSGHYYGFDVWGQGTLVTVSS

SEQ ID NO: 167 - protein, 107 aa, Artificial Sequence
  Synthetic
DIQMTQSPSSLSASVGDRVTITCRASQSIANYLNWYQQKPGKAPKLLIYAASSLQSGVPSRFSGSGSGTDFTLTISSLQPEDFATYYCQQYFSTPPTFGQGTKVEIK

SEQ ID NO: 168 - protein, 121 aa, Artificial Sequence
  Synthetic
EVQLLESGGGLVQPGGSLRLSCAASGFTFGNYAMSWVRQAPGKGLEWVSTISAGGSRTYYADSVKGRFTISRDNSKNTLYLQMNSLRAEDTAVYYCARGWYPILYYAFDYWGQGTLVTVSS

SEQ ID NO: 169 - protein, 107 aa, Artificial Sequence
  Synthetic
DIQMTQSPSSLSASVGDRVTITCRASQSIGNYLNWYQQKPGKAPKLLIYSASSLQSGVPSRFSGSGSGTDFTLTISSLQPEDFATYYCQQTYTIPLTFGQGTKVEIK

SEQ ID NO: 170 - protein, 122 aa, Artificial Sequence
  Synthetic
EVQLLESGGGLVQPGGSLRLSCAASGFTFSSYAMSWVRQAPGKGLEWVSTIYRSGGRTYYADSVKGRFTISRDNSKNTLYLQMNSLRAEDTAVYYCARGAYPLGSYYGFDPWGQGTLVTVSS

SEQ ID NO: 171 - protein, 107 aa, Artificial Sequence
  Synthetic
DIQMTQSPSSLSASVGDRVTITCRASQSIATYLNWYQQKPGKAPKLLIYAASSLQSGVPSRFSGSGSGTDFTLTISSLQPEDFATYYCQQSYTYPVTFGQGTKVEIK

SEQ ID NO: 172 - protein, 121 aa, Artificial Sequence
  Synthetic
EVQLLESGGGLVQPGGSLRLSCAASGFTFSNYAMSWVRQAPGKGLEWVSTIYGSGVRTYYADGVKGRFTISRDNSKNTLYLQMNSLRAEDTAVYYCARGWIPHSFYAFDYWGQGTLVTVSS

SEQ ID NO: 173 - protein, 107 aa, Artificial Sequence
  Synthetic
DIQMTQSPSSLSASVGDRVTITCRASQSISSYLNWYQQKPGKAPKLLIYGASSLQSGVPSRFSGSGSGTDFTLTISSLQPEDFATYYCQQSFTAPVTFGQGTKVEIK

SEQ ID NO: 174 - protein, 121 aa, Artificial Sequence
  Synthetic
EVQLLESGGGLVQPGGSLRLSCAASGFTFTNYAMSWVRQAPGKGLEWVSSISGSGISTYYADSVKGRFTISRDNSKNTLYLQMNSLRAEDTAVYYCARGPGPGYWYAFDVWGQGTLVTVSS

SEQ ID NO: 175 - protein, 107 aa, Artificial Sequence
  Synthetic
DIQMTQSPSSLSASVGDRVTITCRASQTISRYLNWYQQKPGKAPKLLIYAASSLQSGVPSRFSGSGSGTDFTLTISSLQPEDFATYYCQQSYTYPITFGQGTKVEIK

SEQ ID NO: 176 - protein, 120 aa, Artificial Sequence
  Synthetic
QVQLVQSGAEVKKPGSSVKVSCKASGGTFSDEAIHWVRQAPGQGLEWMGRIIPVLGIASYAQKFQGRVTITADESTSTAYMELSSLRSEDTAVYYCARGLSRRAFYFDYWGQGTLVTVSS

SEQ ID NO: 177 - protein, 107 aa, Artificial Sequence; identical to SEQ ID NO: 135
  Synthetic

SEQ ID NO: 178 - protein, 121 aa, Artificial Sequence; identical to SEQ ID NO: 174
  Synthetic

SEQ ID NO: 179 - protein, 107 aa, Artificial Sequence
  Synthetic
DIQMTQSPSSLSASVGDRVTITCRASQTISRYLNWYQQKPGKAPKLLIYSASSLQSGVPSRFSGSGSGTDFTLTISSLQPEDFATYYCQQSYTYPITFGQGTKVEIK

SEQ ID NO: 180 - protein, 121 aa, Artificial Sequence
  Synthetic
EVQLLESGGGLVQPGGSLRLSCAASGFTFSDYAMSWVRQAPGKGLEWVSTIYSGGVRTYYADSVKGRFTISRDNSKNTLYLQMNSLRAEDTAVYYCARGLVSFKGYAFDYWGQGTLVTVSS

SEQ ID NO: 181 - protein, 107 aa, Artificial Sequence; identical to SEQ ID NO: 111
  Synthetic

SEQ ID NO: 182 - protein, 118 aa, Artificial Sequence
  Synthetic
EVQLLESGGGLVQPGGSLRLSCAASGFTFSSYAMSWVRQAPGKGLEWVSAIYGAGGKTYYADSVKGRFTISRDNSKNTLYLQMNSLRAEDTAVYYCARISHGWGFDVWGQGTLVTVSS

SEQ ID NO: 183 - protein, 107 aa, Artificial Sequence
  Synthetic
DIQMTQSPSSLSASVGDRVTITCRASQDISNYLNWYQQKPGKAPKLLIYAASSLQSGVPSRFSGSGSGTDFTLTISSLQPEDFATYYCQQSYTYPLTFGQGTKVEIK

SEQ ID NO: 184 - protein, 120 aa, Artificial Sequence
  Synthetic
EVQLLESGGGLVQPGGSLRLSCAASGFTFSNYAMSWVRQAPGKGLEWVSVIYGSGTRTYYADSVKGRFTISRDNSKNTLYLQMNSLRAEDTAVYYCARVASSYHYAFDYWGQGTLVTVSS

SEQ ID NO: 185 - protein, 107 aa, Artificial Sequence
  Synthetic
DIQMTQSPSSLSASVGDRVTITCRASQSISTYLNWYQQKPGKAPKLLIYAASSLQSGVSSRFSGSGSGTDFTLTISSLQPEDFATYYCQQSYTFPLTFGQGTKVEIK

SEQ ID NO: 186 - protein, 120 aa, Artificial Sequence
  Synthetic
EVQLLESGGGLVQPGGSLRLSCAASGFTFSNYAMSWVRQAPGKGLEWVSVIYGSGGRTYYADSVKGRFTISRDNSKNTLYLQMNSLRAEDTAVYYCARVEDTSYYGMDYWGQGTLVTVSS

SEQ ID NO: 187 - protein, 107 aa, Artificial Sequence
  Synthetic
DIQMTQSPSSLSASVGDRVTITCRASQTIGIYLNWYQQKPGKAPTLLIYAASSLQSGVPSRFSGSGSGTDFTLTISSLQPEDFATYYCQQSYTYPLTFGQGTKVEIK

SEQ ID NO: 188 - protein, 119 aa, Artificial Sequence
  Synthetic
EVQLLESGGGLVQPGGSLRLSCAASGFTFSNYAMSWVRQAPGKGLEWVSAIYGSGGRTYYADSVKGRFTISRDNSKNTLYLQMNSLRAEDTAVYYCARLEAGFYSLDIWGQGTLVTVSS

SEQ ID NO: 189 - protein, 107 aa, Artificial Sequence
  Synthetic
DIQMTQSPSSLSASVGDRVTITCRASQTIGTYLNWYQQKPGKAPKLLIYAASSLQSGVPSRFSGSGSGTDFTLTISSLQPEDFATYYCQQSYSYPLTFGQGTKVEIK

SEQ ID NO: 190 - protein, 119 aa, Artificial Sequence
  Synthetic
EVQLLESGGGLVQPGGSLKLSCAASGFTFGSYAMSWVRQAPGKGLEWVSVISSGGSYTYYADSVKGRFTISRDNSKNTLYLQMNSLRAEDTAVYYCARVSGVVGALDIWGQGTLVTVSS

SEQ ID NO: 191 - protein, 107 aa, Artificial Sequence
  Synthetic
EIVMTQSPATLSVSPGERATLSCRASQSVSNNLAWYQQKPGQAPRLLIYGASTRATGIPARFSGSGSGTEFTLTISSLQSEDFAVYYCQQYNNWPYTFGQGTKVEIK

SEQ ID NO: 192 - protein, 120 aa, Artificial Sequence
  Synthetic
EVQLLESGGGLVQPGGSLRLSCAASGFTFSNYAMSWVRQAPGKGLEWVSTIYSSGSRTYYADSVKGRFTISRDNSKNTLYLQMNSLRAEDTAVYYCARWGGDGFYAMDYWGQGTLVTVSS

SEQ ID NO: 193 - protein, 107 aa, Artificial Sequence
  Synthetic
DIQMTQSPSSLSASVGDRVTITCRASQSISTYLNWYQQKPGKAPKLLIYAASSLQSGVPSRFSGSGSGTDFTLTISSLQPEDFATYYCQQSYTVPLTFGQGTKVEIK

SEQ ID NO: 194 - protein, 121 aa, Artificial Sequence; identical to SEQ ID NO: 180
  Synthetic

SEQ ID NO: 195 - protein, 107 aa, Artificial Sequence; identical to SEQ ID NO: 111
  Synthetic

SEQ ID NO: 196 - protein, 118 aa, Artificial Sequence
  Synthetic
EVQLLESGGGLVQPGGSLRLSCAASGFTFSSYAMSWVRQAPGKGLEWVSAIYGSGGDTYYADSVKGRFTISRDNSKNTLYLQMNSLRAEDTAVYYCARTEYDGYFDVWGQGTLVTVSS

SEQ ID NO: 197 - protein, 107 aa, Artificial Sequence
  Synthetic
DIQMTQSPSSLSASVGDRVTITCRASQDIYNYLNWYQQKPGKAPKLLIYGASSLQSGVPSRFSGSGSGTDFTLTISSLQPEDFATYYCQQSDGFPPTFGQGTKVEIK

SEQ ID NO: 198 - protein, 119 aa, Artificial Sequence
  Synthetic
EVQLLESGGGLVQPGGSLRLSCAASGFTFTDYAMSWVRQAPGKGLEWVSVIYGSGGRTYYADSVKGRFTISRDNSKNTLYLQMNSLRAEDTAVYYCARVEDSHYSFDVWGQGTLVTVSS

SEQ ID NO: 199 - protein, 107 aa, Artificial Sequence
  Synthetic
DIQMTQSPSSLSASVGDRVTITCRASQSISNYLNWYQQKPGKAPKLLIYAASSLQSGVPSRFSGSGSGTDFTLTISSLQPEDFATYYCQQSYTVPWTFGQGTKVEIK

SEQ ID NO: 200 - protein, 126 aa, Artificial Sequence
  Synthetic
QVQLVESGGGVVQPGRSLRLSCAASGFPFSDYAMHWLRQAPGKGLEWVAVISYDGNIEYYANSVKGRFTISRDNSKNTLYLQMNSLRAEDTAVYYCARDRIGSSTYYGYYNGFDVWGQGTLVTVSS

SEQ ID NO: 201 - protein, 107 aa, Artificial Sequence
  Synthetic
EIVMTQSPATLSVSPGERATLSCRASQSVSSNLAWYQQKPGQAPRLLIYGASTRATGIPARFSGSGSGTEFTLTISSLQSEDFAVYYCQQYNDYPLTFGQGTKVEIK

SEQ ID NO: 202 - protein, 117 aa, Artificial Sequence
  Synthetic
EVQLLESGGGLVQPGGSLRLSCAASGFTFSNYAMSWVRQAPGKGLEWVSAIKGSGGGTYYADSVKGRFTISRDNSKNTLYLQMNSLRAEDTAVYYCARGGYLWLDPWGQGTLVTVSS

SEQ ID NO: 203 - protein, 107 aa, Artificial Sequence
  Synthetic
EIVMTQSPATLSVSPGERATLSCRASQSISSNLAWYQQKPGQAPRLLIYDASTRATGIPARFSGSGSGTEFTLTISSLQSEDFAVYYCQQYYNWPLTFGQGTKVEIK

SEQ ID NO: 204 - protein, 125 aa, Artificial Sequence
  Synthetic
EVQLLESGGGLVQPGGSLRLSCAASGFTFSNYAMSWVRQAPGKGLEWVSAISGSGTYTYYADSVKGRFTISRDNSKNTLYLQMNSLRAEDTAVYYCARVRISGYGPGYFGGFDVWGQGTLVTVSS

SEQ ID NO: 205 - protein, 107 aa, Artificial Sequence
  Synthetic
EIVMTQSPATLSVSPGERATLSCRASQSVYSNLAWYQQKPGQAPRLLIYGASTRATGIPARFSGSGSGTEFTLTISSLQSEDFAVYYCQQYSNWPLTFGQGTKVEIK

SEQ ID NO: 206 - protein, 125 aa, Artificial Sequence
  Synthetic
EVQLLESGGGLVQPGGSLRLSCAASGFTFSRYAMSWVRQAPGKGLEWVSIISGTGGNTYYADSVKGRFTISRDNSKNTLYLQMNSLRAEDTAVYYCARIDTFAYYYSLYRAFDLWGQGTLVTVSS

SEQ ID NO: 207 - protein, 107 aa, Artificial Sequence
  Synthetic
EIVMTQSPATLSVSPGERATLSCRASQSVGGNLAWYQQKPGQAPRLLIYDASTRATGIPARFSGSGSGTEFTLTISSLQSEDFAVYYCQQYNDWPLTFGQGTKVEIK

SEQ ID NO: 208 - protein, 124 aa, Artificial Sequence
  Synthetic
EVQLLESGGGLVQPGGSLRLSCAASGFTFSDYAMSWVRQAPGKGLEWVSSISEGGGSTYYADSVKGRFTISRDNSKNTLYLQMNSLRAEDTAVYYCARTKRYFAGGSYYWMDVWGQGTLVTVSS

SEQ ID NO: 209 - protein, 107 aa, Artificial Sequence
  Synthetic
DIQMTQSPSSLSASVGDRVTITCRASQRISSYLNWYQQKPGKAPKLLIYAASSLQSGVPSRFSGSGSGTDFALTISSLQPEDFATYYCQQSYSYPITFGQGTKVEIK

SEQ ID NO: 210 - protein, 120 aa, Artificial Sequence
  Synthetic
EVQLLESGGGLVQPGGSLRLSCAASGFTFSSYAMSWVRQAPGKGLEWVSVISGGSGHTTYYADSVKGRFTISRDNSKNTLYLQMNSLRAEDTAVYYCARHGGGRFAMDVWGQGTLVTVSS

SEQ ID NO: 211 - protein, 108 aa, Artificial Sequence
  Synthetic
EIVLTQSPGTLSLSPGERATLSCRASQSVASPYLAWYQQKPGQAPRLLIYGASSRATGIPDRFSGSGSGTDFTLTISRLEPEDFAVYYCQQYYESPITFGQGTKVEIK

SEQ ID NO: 212 - protein, 127 aa, Artificial Sequence
  Synthetic
EVQLLESGGGLVQPGGSLRLSCAASGFTFSTYAMSWVRQAPGKGLEWVSSISGSGGKTYYADSVKGRFTISRDNSKNTLYLQMNSLRAEDTAVYYCARDIYGITYDGYTYYDGMDVWGQGTLVTVSS

SEQ ID NO: 213 - protein, 107 aa, Artificial Sequence
  Synthetic
DIQMTQSPSSLSASVGDRVTITCRASQSIYNYLNWHQQKPGKAPKLLIYAASSLQSGVPSRFSGSGSGTDFTLTISSLQPEDFATYYCQQSDSPPWTFGQGTKVEIK

SEQ ID NO: 214 - protein, 118 aa, Artificial Sequence; identical to SEQ ID NO: 106
  Synthetic

SEQ ID NO: 215 - protein, 109 aa, Artificial Sequence; identical to SEQ ID NO: 107
  Synthetic

SEQ ID NO: 216 - protein, 123 aa, Artificial Sequence
  Synthetic
EVQLLESGGGLVQPGGSLRLSCAASGFTFSNYAMSWVRQAPGKGLEWVSTISGSGANTYYADSVKGRFTISRDNSKNTLYLQMNSLRAEDTAVYYCARHHDIAPSWIYYFDYWGQGTLVTVSS

SEQ ID NO: 217 - protein, 108 aa, Artificial Sequence
  Synthetic
EIVLTQSPGTLSLSPGERATLSCRARQSVPSNYLAWYQQKPGQAPRLLIYGASSRATGIPDRFSGSGSGTDFTLTISRLEPEDFAVYYCQQYYSSPLTFGQGTKVEIK

SEQ ID NO: 218 - protein, 121 aa, Artificial Sequence
  Synthetic
EVQLLESGGGLVQPGGSLRLSCAASGFTFPDYAMSWVRQAPGKGLEWVSTIYAGGGRTYYADSVKGRFTISRDNSKNTLYLQMNSLRAEDTAVYYCARGLTRHYGYSFDHWGQGTLVTVSS

SEQ ID NO: 219 - protein, 107 aa, Artificial Sequence
  Synthetic
DIQMTQSPSSLSASVGDRVTITCRASQTISSYLNWYQQKPGKAPKLLIYSASSLQSGVPSRFSGSGSGTDFTLTISSLQPEDFATYYCQQSYTFPLTFGQGTKVEIK

SEQ ID NO: 220 - protein, 120 aa, Artificial Sequence
  Synthetic
EVQLLESGGGLVQPGGSLRLSCAASGFTFSNYAMSWVRQAPGKGLEWVSSIYGSGHRTYYADSVKGRFTISRDNSKNTLYLQMNSLRAEDTAVYYCARVASSAEYAFDHWGQGTLVTVSS

SEQ ID NO: 221 - protein, 107 aa, Artificial Sequence
  Synthetic
DIQMTQSPSSLSASVGDRVTITCRASQSISNYLNWYQQKSGKAPKLLIYAASSLQSGVPSRFSGSGSGTDFTLTISSLQPEDFATYYCQQSYTVPLTFGQGTKVEIK

SEQ ID NO: 222 - protein, 126 aa, Artificial Sequence; identical to SEQ ID NO: 46
  Synthetic

SEQ ID NO: 223 - protein, 109 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSPTYTFGQGTKVEIK

SEQ ID NO: 224 - protein, 118 aa, Artificial Sequence
  Synthetic
QVQLVESGGGVVQPGRSLRLSCAASGFSFSDYGMHWVRQAPGKGLEWVAVISYEGSNEYYADSVKGRFTISRDNSKNTLYLQMNSLRAEDTAVYYCARGSEGSGLDVWGQGTLVTVSS

SEQ ID NO: 225 - protein, 107 aa, Artificial Sequence
  Synthetic
DIQMTQSPSSLSASVGDRVTITCRASQRIYNYLNWYQQKPGKAPKLLIYAASSLQSGVPSRFSGSSSGTDFTLTISSLQPEDFATYYCQQAYSPPYTFGQGTKVEIK

SEQ ID NO: 226 - protein, 120 aa, Artificial Sequence
  Synthetic
EVQLLESGGGLVQPGGSLRLSCAASGFTFSNYAMSWVRQAPGKGLEWVSAISGGGSRTFYADSVKGRFTISRDNSKNTLYLQMDSLRAEDTAVYYCAREARVLGYSFDYWGQGTLVTVSS

SEQ ID NO: 227 - protein, 107 aa, Artificial Sequence
  Synthetic
DIQMTQSPSSLSASVGDRVTITCRASQRIGTYLNWYQQKPGKAPKLLIYAASSLQSGVPSRFSGSGSGTDFTLTISSLQPEDFATYYCQQSYSYPLTFGQGTKVEIK

SEQ ID NO: 228 - protein, 118 aa, Artificial Sequence
  Synthetic
QVQLVESGGGVVQPGRSLRLSCAASGFTFSDYGMHWVRQAPGKGLEWVAVISYEGSNEYYADSVKGRFTISRDNSKNTLYLQMNSLRAEDTAVYYCARGSDGDGFDVWGQGTLVTVSS

SEQ ID NO: 229 - protein, 107 aa, Artificial Sequence
  Synthetic
DIQMTQSPSSLSASVGDRVTITCRASQRIANYLNWYQQKPGKAPKLLIYAASSLQSGVPSRFSGSGSGTDFTLTISSLQPEDFATYYCQQSYSPPYTFGQGTKVEIK

SEQ ID NO: 230 - protein, 118 aa, Artificial Sequence
  Synthetic
QVQLVESGGGVVQPGRSLRLSCAASGFTFSDYGMHWVRQAPGKGLEWVAVISYEGSREYYADSVKGRFTISRDNSKNTLYLQMNSLRAEDTAVYYCARGSEGDGFDVWGQGTLVTVSS

SEQ ID NO: 231 - protein, 107 aa, Artificial Sequence
  Synthetic
DIQMTQSPSSLSASVGDRVTITCRASQRISNYLNWYQQKPGKAPKLLIYAASSLQSGVPSRFSGSGSGTDFTLTISSLQPEDFATYYCQQSYSAPYTFGQGTKVEIK

SEQ ID NO: 232 - protein, 118 aa, Artificial Sequence
  Synthetic
QVQLVESGGGVVQPGRSLRLSCAASGFTFDDYGMHWVRQAPGKGLEWVAVISYEGSNQYYADSVKGRFTISRDNSKNTLYLQMNSLRAEDTAVYYCARGSDGSGFDVWGQGTLVTVSS

SEQ ID NO: 233 - protein, 107 aa, Artificial Sequence
  Synthetic
DIQMTQSPSSLSASVGDRVTITCRASQDISNYLNWYQQKPGKAPKLLIYAASSLQSGVPSRFSGSGSGTDFTLTISSLQPEDFATYYCQQAYSPPYTFGQGTKVEIK

SEQ ID NO: 234 - protein, 118 aa, Artificial Sequence
  Synthetic
QVQLVESGGGVVQPGRSLRLSCAASGFAFSDYGMHWVRQAPGKGLEWVAVISYEGSREYYADSVKGRFTISRDNSKNTLYLQMNSLRAEDTAVYYCARGSEGDGFDVWGQGTLVTVSS

SEQ ID NO: 235 - protein, 107 aa, Artificial Sequence
  Synthetic
DIQMTQSPSSLSASVGDRVTITCRASQRIYNYLNWYQQKPGKAPKLLIYAASSLQSGVPSRFSGSGSGTDFTLTISSLQPEDFATYYCQQSYSPPYTFGQGTKVEIK

SEQ ID NO: 236 - protein, 118 aa, Artificial Sequence
  Synthetic
QVQLVESGGGVVQPGRSLRLSCAASGFAFSDYGMHWVRQAPGKGLEWVAVISYEGNNEYYADSVKGRFTISRDNSKNTLYLQMNSLRAEDTAVYYCARGSDGDGFDVWGQGTLVTVSS

SEQ ID NO: 237 - protein, 107 aa, Artificial Sequence
  Synthetic
DIQMTQSPSSLSASVGDRVTITCRASQRIGNYLNWYQQKPGKAPKLLIYAASSLQSGVPSRFSGSGSGTDFTLTISSLQPEDFATYYCQQSYSPPYTFGQGTKVEIK

SEQ ID NO: 238 - protein, 120 aa, Artificial Sequence
  Synthetic
EVQLLESGGGLVQPGGSLRLSCAASGFTFSAYAMSWVRQAPGKGLEWVSTISGGGSRTFYADSVKGRFTISRDNSKNTLYLQMNSLRAEDTAVYYCAREAVVLGYSLDYWGQGTLVTVSS

SEQ ID NO: 239 - protein, 107 aa, Artificial Sequence
  Synthetic
DIQMTQSPSSLSASVGDRVTITCRASQGIGKYLNWYQQKPGKAPKLLIYAASSLQSGVPSRFSGSGSGTDFTLTISSLQPEDFATYYCQQSYSYPLTFGQGTKVEIK

SEQ ID NO: 240 - protein, 120 aa, Artificial Sequence
  Synthetic
EVQLLESGGGLVQPGGSLRLSCAASGFTFSNYAMSWVRQAPGKGLEWVSIISGGGSRTYYADSVKGRFTISRDNSKNTLYLQMNSLRAEDTAVYYCAREARVLGYSLDYWGQGTLVTVSS

SEQ ID NO: 241 - protein, 107 aa, Artificial Sequence
  Synthetic
DIQMTQSPSSLSASVGDRVTITCRASQRISKYLNWYQQKPGKAPKLLIYAASSLQSGVPSRFSGSGSGTDFTLTISSLQPEDFATYYCQQSFSYPLTFGQGTKVEIK

SEQ ID NO: 242 - protein, 120 aa, Artificial Sequence
  Synthetic
EVQLLESGGGLVQPGGSLRLSCAASGFTFSGYAMSWVRQAPGKGLEWVSIISGGGSRTYYADSVKGRFTISRDNSKNTLYLQMNSLRAEDTAVYYCAREARVLGYSLDYWGQGTLVTVSS

SEQ ID NO: 243 - protein, 107 aa, Artificial Sequence
  Synthetic
DIQMTQSPSSLSASVGDRVTITCRASQRISKYLNWYQQKPGKAPKLLIYAASSLQSGVPSRFSGSGSGTDFTLTISSLQPEDFATYYCQQSYIYPLTFGQGTKVEIK

SEQ ID NO: 244 - protein, 120 aa, Artificial Sequence
  Synthetic
EVQLLESGGGLVQPGGSLRLSCAASGFTFSTYAMSWVRQAPGKGLEWVSIISGGGSRTYYADSVKGRFTVSRDNSKNTLYLQMNSLRAEDTAVYYCAREHRVLGYSLDYWGQGTLVTVSS

SEQ ID NO: 245 - protein, 107 aa, Artificial Sequence; identical to SEQ ID NO: 243
  Synthetic

SEQ ID NO: 246 - protein, 120 aa, Artificial Sequence
  Synthetic
EVQLLESGGGLVQPGGSLRLSCAASGFTFGNYAMSWVRQAPGKGLEWVSIISGGGSRTYYADSVKGRFTISRDNSKNTLYLQMNSLRAEDTAVYYCAREARVLGYSLDYWGQGTLVTVSS

SEQ ID NO: 247 - protein, 107 aa, Artificial Sequence; identical to SEQ ID NO: 243
  Synthetic

SEQ ID NO: 248 - protein, 120 aa, Artificial Sequence
  Synthetic
EVQLLESGGGLVQPGGSLRLSCAASGFTFGNYAMSWVRQAPGKGLEWVSAISGGGSRTFYADSVKGRFTISRDNSKNTLYLQMNSLRAEDTAVYYCAREARVLFYSLDYWGQGTLVTVSS

SEQ ID NO: 249 - protein, 107 aa, Artificial Sequence
  Synthetic
DIQMTQSPSSLSASVGDRVTITCRASQRIGIYLNWYQQKPGKAPKLLIYAASSLQSGVPSRFSGSGSGTDFTLTISSLQPEDFATYYCQQSYSYPLTFGQGTKVEIK

SEQ ID NO: 250 - protein, 118 aa, Artificial Sequence; identical to SEQ ID NO: 230
  Synthetic

SEQ ID NO: 251 - protein, 107 aa, Artificial Sequence
  Synthetic
DIQMTQSPSSLSASVGDRVTITCRASQRIYNYLNWYQQKPGKAPKLLIYAASSLQSGVPSRFSGSGSGTDFTLTISSLQPEDFATYYCQQGYSPPYTFGQGTKVEIK

SEQ ID NO: 252 - protein, 118 aa, Artificial Sequence
  Synthetic
QVQLVESGGGVVQPGRSLRLSCAASGFIFSDYGMHWVRQAPGKGLEWVAVISYEGSREYYADSVKGRFTISRDNSKNTLYLQMNSLRAEDTAVYYCARGSEGDGFDVWGQGTLVTVSS

SEQ ID NO: 253 - protein, 107 aa, Artificial Sequence
  Synthetic
DIQMTQSPSSLSASVGDRVTITCRASQRISSYLNWYQQKPGKAPKLLIYAASSLQSGVPSRFSGSGSGTDFTLTISSLQPEDFATYYCQQSYSAPYTFGQGTKVEIK

SEQ ID NO: 254 - protein, 118 aa, Artificial Sequence
  Synthetic
QVQLVESGGGVVQPGRSLRLSCAASGFTFDDYGMHWVRQAPGKGLEWVAVISYEGSNQYYADSVKGRFTISRDNSKNTLYLQMNSLRAEDTAVYYCARGSEGDGFDVWGQGTLVTVSS

SEQ ID NO: 255 - protein, 107 aa, Artificial Sequence
  Synthetic
DIQMTQSPSSLSASVGDRVTITCRASQRIGNYLNWYQQKPGKAPKLLIYAASSLQSGVPSRFSGSGSGTDFTLTISSLQPEDFATYYCQQGYSPPYTFGQGTKVEIK

SEQ ID NO: 256 - protein, 118 aa, Artificial Sequence; identical to SEQ ID NO: 252
  Synthetic

SEQ ID NO: 257 - protein, 107 aa, Artificial Sequence
  Synthetic
DIQMTQSPSSLSASVGDRVTITCRASQRISNYLNWYQQKPGKAPKLLIYAASSLQSGVPSRFSGSGSGTDFTLTISSLQPEDFATYYCQQGYSPPYTFGQGTKVEIK

SEQ ID NO: 258 - protein, 118 aa, Artificial Sequence; identical to SEQ ID NO: 252
  Synthetic

SEQ ID NO: 259 - protein, 107 aa, Artificial Sequence
  Synthetic
DIQMTQSPSSLSASVGDRVTITCRASQRISKYLNWYQQKPGKAPKLLIYAASSLQSGVPSRFSGSGSGTDFTLTISSLQPEDFATYYCQQSYSPPYTFGQGTKVEIK

SEQ ID NO: 260 - protein, 118 aa, Artificial Sequence
  Synthetic
QVQLVESGGGVVQPGRSLRLSCAASGFTFGDYGMHWVRQAPGKGLEWVAVISYEGSREYYADSVKGRFTISRDNSKNTLYLQMNSLRAEDTAVYYCARGSEGDGFDVWGQGTLVTVSS

SEQ ID NO: 261 - protein, 107 aa, Artificial Sequence; identical to SEQ ID NO: 235
  Synthetic

SEQ ID NO: 262 - protein, 118 aa, Artificial Sequence; identical to SEQ ID NO: 252
  Synthetic

SEQ ID NO: 263 - protein, 107 aa, Artificial Sequence
  Synthetic
DIQMTQSPSSLSASVGDRVTITCRASQTISNYLNWYQQKPGKAPKLLIYAASSLQSGVPSRFSGSGSGTDFTLTISSLQPEDFATYYCQQSYSPPYTFGQGTKVEIK

SEQ ID NO: 264 - protein, 120 aa, Artificial Sequence
  Synthetic
EVQLLESGGGLVQPGGSLRLSCAASGFTFSNYAMSWVRQAPGKGLEWVSAISGGGSYTYYADSVKGRFTISRDNSKNTLYLQMNSLRAEDTAVYYCAREERVLGYSLDYWGQGTLVTVSS

SEQ ID NO: 265 - protein, 107 aa, Artificial Sequence
  Synthetic
DIQMTQSPSSLSASVGDRVTITCRASQRIYKYLNWYQQKPGKAPKLLIYAASSLQSGVPSRFSGSGSGTDFTLTISSLQPEDFATYYCQQSFSYPLTFGQGTKVEIK

SEQ ID NO: 266 - protein, 5 aa, Artificial Sequence
  Synthetic
SYAMS

SEQ ID NO: 267 - protein, 17 aa, Artificial Sequence
  Synthetic
TIYASGATTYYADSVKG

SEQ ID NO: 268 - protein, 10 aa, Artificial Sequence
  Synthetic
LKLYHAAFDI

SEQ ID NO: 269 - protein, 11 aa, Artificial Sequence
  Synthetic
RASQDISSYLN

SEQ ID NO: 270 - protein, 7 aa, Artificial Sequence
  Synthetic
TASSLQS

SEQ ID NO: 271 - protein, 9 aa, Artificial Sequence
  Synthetic
QQSYSYPLT

SEQ ID NO: 272 - protein, 5 aa, Artificial Sequence
  Synthetic
NYAMS

SEQ ID NO: 273 - protein, 17 aa, Artificial Sequence
  Synthetic
AISASGGYTYYADSVKG

SEQ ID NO: 274 - protein, 8 aa, Artificial Sequence
  Synthetic
GRWGAFDY

SEQ ID NO: 275 - protein, 14 aa, Artificial Sequence
  Synthetic
TGSSSNIGSGYDVH

SEQ ID NO: 276 - protein, 7 aa, Artificial Sequence
  Synthetic
GNRNRPS

SEQ ID NO: 277 - protein, 11 aa, Artificial Sequence
  Synthetic
QSYDSSLKTWV

SEQ ID NO: 278 - protein, 5 aa, Artificial Sequence
  Synthetic
DYGMH

SEQ ID NO: 279 - protein, 17 aa, Artificial Sequence
  Synthetic
VISYHGRNEYYANSVKG

SEQ ID NO: 280 - protein, 9 aa, Artificial Sequence
  Synthetic
GAYGDDFDV

SEQ ID NO: 281 - protein, 11 aa, Artificial Sequence
  Synthetic
RASQSISSYLN

SEQ ID NO: 282 - protein, 7 aa, Artificial Sequence
  Synthetic
AASSLQS

SEQ ID NO: 283 - protein, 9 aa, Artificial Sequence
  Synthetic
QQSYSAPYT

SEQ ID NO: 284 - protein, 5 aa, Artificial Sequence
  Synthetic
DYGMH

SEQ ID NO: 285 - protein, 17 aa, Artificial Sequence
  Synthetic
VISYEGSNEYYADSVKG

SEQ ID NO: 286 - protein, 9 aa, Artificial Sequence
  Synthetic
GSEGSGFDV

SEQ ID NO: 287 - protein, 11 aa, Artificial Sequence
  Synthetic
RASQRISNYLN

SEQ ID NO: 288 - protein, 7 aa, Artificial Sequence
  Synthetic
AASSLQS

SEQ ID NO: 289 - protein, 9 aa, Artificial Sequence
  Synthetic
QQSYSPPYT

SEQ ID NO: 290 - protein, 5 aa, Artificial Sequence
  Synthetic
DYGMH

SEQ ID NO: 291 - protein, 17 aa, Artificial Sequence
  Synthetic
VISYDGSRKYYANSVKG

SEQ ID NO: 292 - protein, 9 aa, Artificial Sequence
  Synthetic
GSYSDGFDV

SEQ ID NO: 293 - protein, 11 aa, Artificial Sequence
  Synthetic
RASQSISRYLN

SEQ ID NO: 294 - protein, 7 aa, Artificial Sequence
  Synthetic
GASSLQS

SEQ ID NO: 295 - protein, 9 aa, Artificial Sequence
  Synthetic
QQSYSTPYT

SEQ ID NO: 296 - protein, 5 aa, Artificial Sequence
  Synthetic
TEYIS

SEQ ID NO: 297 - protein, 17 aa, Artificial Sequence
  Synthetic
RIIPVLGITNYAQKFQG

SEQ ID NO: 298 - protein, 16 aa, Artificial Sequence
  Synthetic
HLGPPYLVTYSHGFDV

SEQ ID NO: 299 - protein, 11 aa, Artificial Sequence
  Synthetic
RASQSLGTNLA

SEQ ID NO: 300 - protein, 7 aa, Artificial Sequence
  Synthetic
GASTRAT

SEQ ID NO: 301 - protein, 9 aa, Artificial Sequence
  Synthetic
QQYYEAPPT

SEQ ID NO: 302 - protein, 5 aa, Artificial Sequence
  Synthetic
NYAMS

SEQ ID NO: 303 - protein, 17 aa, Artificial Sequence
  Synthetic
SISGGGINTYYADSVKG

SEQ ID NO: 304 - protein, 12 aa, Artificial Sequence
  Synthetic
VRSYSGPGYFDY

SEQ ID NO: 305 - protein, 11 aa, Artificial Sequence
  Synthetic
RASRSVSSNLA

SEQ ID NO: 306 - protein, 7 aa, Artificial Sequence
  Synthetic
GASTRAT

SEQ ID NO: 307 - protein, 9 aa, Artificial Sequence
  Synthetic
QQYYNWPLT

SEQ ID NO: 308 - protein, 5 aa, Artificial Sequence
  Synthetic
NYAMS

SEQ ID NO: 309 - protein, 17 aa, Artificial Sequence
  Synthetic
AISGGGSRTYYADSVKG

SEQ ID NO: 310 - protein, 11 aa, Artificial Sequence
  Synthetic
EARVLGYSLDY

SEQ ID NO: 311 - protein, 11 aa, Artificial Sequence
  Synthetic
RASQRIGKYLN

SEQ ID NO: 312 - protein, 7 aa, Artificial Sequence
  Synthetic
AASSLQS

SEQ ID NO: 313 - protein, 9 aa, Artificial Sequence
  Synthetic
QQSYSYPLT

SEQ ID NO: 314 - protein, 5 aa, Artificial Sequence
  Synthetic
SYAMS

SEQ ID NO: 315 - protein, 17 aa, Artificial Sequence
  Synthetic
LISSSGENTYYADSVKG

SEQ ID NO: 316 - protein, 16 aa, Artificial Sequence
  Synthetic
VRFGYGSWRYRKYMDV

SEQ ID NO: 317 - protein, 11 aa, Artificial Sequence
  Synthetic
RASQSIATYLN

SEQ ID NO: 318 - protein, 7 aa, Artificial Sequence
  Synthetic
GASSLQS

SEQ ID NO: 319 - protein, 9 aa, Artificial Sequence
  Synthetic
QQSYSTPLT

SEQ ID NO: 320 - protein, 5 aa, Artificial Sequence
  Synthetic
DEAIH

SEQ ID NO: 321 - protein, 17 aa, Artificial Sequence
  Synthetic
RIIPVLGIASYAQKFQG

SEQ ID NO: 322 - protein, 11 aa, Artificial Sequence
  Synthetic
GLSRRAFYFDY

SEQ ID NO: 323 - protein, 11 aa, Artificial Sequence
  Synthetic
RASQTIGNYLN

SEQ ID NO: 324 - protein, 7 aa, Artificial Sequence
  Synthetic
VASSLQS

SEQ ID NO: 325 - protein, 9 aa, Artificial Sequence
  Synthetic
QQSYRYPYT

SEQ ID NO: 326 - protein, 5 aa, Artificial Sequence
  Synthetic
DYGMH

SEQ ID NO: 327 - protein, 17 aa, Artificial Sequence
  Synthetic
VISYEGSNEYYADSVKG

SEQ ID NO: 328 - protein, 9 aa, Artificial Sequence
  Synthetic
GSEGSGLDV

SEQ ID NO: 329 - protein, 11 aa, Artificial Sequence
  Synthetic
RASQRIYNYLN

SEQ ID NO: 330 - protein, 7 aa, Artificial Sequence
  Synthetic
AASSLQS

SEQ ID NO: 331 - protein, 9 aa, Artificial Sequence
  Synthetic
QQAYSPPYT

SEQ ID NO: 332 - protein, 5 aa, Artificial Sequence
  Synthetic
NYAMS

SEQ ID NO: 333 - protein, 17 aa, Artificial Sequence
  Synthetic
AISGGGSRTFYADSVKG

SEQ ID NO: 334 - protein, 11 aa, Artificial Sequence
  Synthetic
EARVLGYSFDY

SEQ ID NO: 335 - protein, 11 aa, Artificial Sequence
  Synthetic
RASQRIGTYLN

SEQ ID NO: 336 - protein, 7 aa, Artificial Sequence
  Synthetic
AASSLQS

SEQ ID NO: 337 - protein, 9 aa, Artificial Sequence
  Synthetic
QQSYSYPLT

SEQ ID NO: 338 - protein, 5 aa, Artificial Sequence
  Synthetic
DYGMH

SEQ ID NO: 339 - protein, 17 aa, Artificial Sequence
  Synthetic
VISYEGSNEYYADSVKG

SEQ ID NO: 340 - protein, 9 aa, Artificial Sequence
  Synthetic
GSDGDGFDV

SEQ ID NO: 341 - protein, 11 aa, Artificial Sequence
  Synthetic
RASQRIANYLN

SEQ ID NO: 342 - protein, 7 aa, Artificial Sequence
  Synthetic
AASSLQS

SEQ ID NO: 343 - protein, 9 aa, Artificial Sequence
  Synthetic
QQSYSPPYT

SEQ ID NO: 344 - protein, 5 aa, Artificial Sequence
  Synthetic
DYGMH

SEQ ID NO: 345 - protein, 17 aa, Artificial Sequence
  Synthetic
VISYEGSREYYADSVKG

SEQ ID NO: 346 - protein, 9 aa, Artificial Sequence
  Synthetic
GSEGDGFDV

SEQ ID NO: 347 - protein, 11 aa, Artificial Sequence
  Synthetic
RASQRISNYLN

SEQ ID NO: 348 - protein, 7 aa, Artificial Sequence
  Synthetic
AASSLQS

SEQ ID NO: 349 - protein, 9 aa, Artificial Sequence
  Synthetic
QQSYSAPYT

SEQ ID NO: 350 - protein, 5 aa, Artificial Sequence
  Synthetic
DYGMH

SEQ ID NO: 351 - protein, 17 aa, Artificial Sequence
  Synthetic
VISYEGSNQYYADSVKG

SEQ ID NO: 352 - protein, 9 aa, Artificial Sequence
  Synthetic
GSDGSGFDV

SEQ ID NO: 353 - protein, 11 aa, Artificial Sequence
  Synthetic
RASQDISNYLN

SEQ ID NO: 354 - protein, 7 aa, Artificial Sequence
  Synthetic
AASSLQS

SEQ ID NO: 355 - protein, 9 aa, Artificial Sequence
  Synthetic
QQAYSPPYT

SEQ ID NO: 356 - protein, 5 aa, Artificial Sequence
  Synthetic
DYGMH

SEQ ID NO: 357 - protein, 17 aa, Artificial Sequence
  Synthetic
VISYEGSREYYADSVKG

SEQ ID NO: 358 - protein, 9 aa, Artificial Sequence
  Synthetic
GSEGDGFDV

SEQ ID NO: 359 - protein, 11 aa, Artificial Sequence
  Synthetic
RASQRIYNYLN

SEQ ID NO: 360 - protein, 7 aa, Artificial Sequence
  Synthetic
AASSLQS

SEQ ID NO: 361 - protein, 9 aa, Artificial Sequence
  Synthetic
QQSYSPPYT

SEQ ID NO: 362 - protein, 5 aa, Artificial Sequence
  Synthetic
DYGMH

SEQ ID NO: 363 - protein, 17 aa, Artificial Sequence
  Synthetic
VISYEGNNEYYADSVKG

SEQ ID NO: 364 - protein, 9 aa, Artificial Sequence
  Synthetic
GSDGDGFDV

SEQ ID NO: 365 - protein, 11 aa, Artificial Sequence
  Synthetic
RASQRIGNYLN

SEQ ID NO: 366 - protein, 7 aa, Artificial Sequence
  Synthetic
AASSLQS

SEQ ID NO: 367 - protein, 9 aa, Artificial Sequence
  Synthetic
QQSYSPPYT

SEQ ID NO: 368 - protein, 5 aa, Artificial Sequence
  Synthetic
AYAMS

SEQ ID NO: 369 - protein, 17 aa, Artificial Sequence
  Synthetic
TISGGGSRTFYADSVKG

SEQ ID NO: 370 - protein, 11 aa, Artificial Sequence
  Synthetic
EAVVLGYSLDY

SEQ ID NO: 371 - protein, 11 aa, Artificial Sequence
  Synthetic
RASQGIGKYLN

SEQ ID NO: 372 - protein, 7 aa, Artificial Sequence
  Synthetic
AASSLQS

SEQ ID NO: 373 - protein, 9 aa, Artificial Sequence
  Synthetic
QQSYSYPLT

SEQ ID NO: 374 - protein, 5 aa, Artificial Sequence
  Synthetic
NYAMS

SEQ ID NO: 375 - protein, 17 aa, Artificial Sequence
  Synthetic
IISGGGSRTYYADSVKG

SEQ ID NO: 376 - protein, 11 aa, Artificial Sequence
  Synthetic
EARVLGYSLDY

SEQ ID NO: 377 - protein, 11 aa, Artificial Sequence
  Synthetic
RASQRISKYLN

SEQ ID NO: 378 - protein, 7 aa, Artificial Sequence
  Synthetic
AASSLQS

SEQ ID NO: 379 - protein, 9 aa, Artificial Sequence
  Synthetic
QQSFSYPLT

SEQ ID NO: 380 - protein, 5 aa, Artificial Sequence
  Synthetic
GYAMS

SEQ ID NO: 381 - protein, 17 aa, Artificial Sequence
  Synthetic
IISGGGSRTYYADSVKG

SEQ ID NO: 382 - protein, 11 aa, Artificial Sequence
  Synthetic
EARVLGYSLDY

SEQ ID NO: 383 - protein, 11 aa, Artificial Sequence
  Synthetic
RASQRISKYLN

SEQ ID NO: 384 - protein, 7 aa, Artificial Sequence
  Synthetic
AASSLQS

SEQ ID NO: 385 - protein, 9 aa, Artificial Sequence
  Synthetic
QQSYIYPLT

SEQ ID NO: 386 - protein, 5 aa, Artificial Sequence
  Synthetic
TYAMS

SEQ ID NO: 387 - protein, 17 aa, Artificial Sequence
  Synthetic
IISGGGSRTYYADSVKG

SEQ ID NO: 388 - protein, 11 aa, Artificial Sequence
  Synthetic
EHRVLGYSLDY

SEQ ID NO: 389 - protein, 11 aa, Artificial Sequence
  Synthetic
RASQRISKYLN

SEQ ID NO: 390 - protein, 7 aa, Artificial Sequence
  Synthetic
AASSLQS

SEQ ID NO: 391 - protein, 9 aa, Artificial Sequence
  Synthetic
QQSYIYPLT

SEQ ID NO: 392 - protein, 5 aa, Artificial Sequence
  Synthetic
NYAMS

SEQ ID NO: 393 - protein, 17 aa, Artificial Sequence
  Synthetic
IISGGGSRTYYADSVKG

SEQ ID NO: 394 - protein, 11 aa, Artificial Sequence
  Synthetic
EARVLGYSLDY

SEQ ID NO: 395 - protein, 11 aa, Artificial Sequence
  Synthetic
RASQRISKYLN

SEQ ID NO: 396 - protein, 7 aa, Artificial Sequence
  Synthetic
AASSLQS

SEQ ID NO: 397 - protein, 9 aa, Artificial Sequence
  Synthetic
QQSYIYPLT

SEQ ID NO: 398 - protein, 5 aa, Artificial Sequence
  Synthetic
NYAMS

SEQ ID NO: 399 - protein, 17 aa, Artificial Sequence
  Synthetic
AISGGGSRTFYADSVKG

SEQ ID NO: 400 - protein, 11 aa, Artificial Sequence
  Synthetic
EARVLFYSLDY

SEQ ID NO: 401 - protein, 11 aa, Artificial Sequence
  Synthetic
RASQRIGIYLN

SEQ ID NO: 402 - protein, 7 aa, Artificial Sequence
  Synthetic
AASSLQS

SEQ ID NO: 403 - protein, 9 aa, Artificial Sequence
  Synthetic
QQSYSYPLT

SEQ ID NO: 404 - protein, 5 aa, Artificial Sequence
  Synthetic
DYGMH

SEQ ID NO: 405 - protein, 17 aa, Artificial Sequence
  Synthetic
VISYEGSREYYADSVKG

SEQ ID NO: 406 - protein, 9 aa, Artificial Sequence
  Synthetic
GSEGDGFDV

SEQ ID NO: 407 - protein, 11 aa, Artificial Sequence
  Synthetic
RASQRIYNYLN

SEQ ID NO: 408 - protein, 7 aa, Artificial Sequence
  Synthetic
AASSLQS

SEQ ID NO: 409 - protein, 9 aa, Artificial Sequence
  Synthetic
QQGYSPPYT

SEQ ID NO: 410 - protein, 5 aa, Artificial Sequence
  Synthetic
DYGMH

SEQ ID NO: 411 - protein, 17 aa, Artificial Sequence
  Synthetic
VISYEGSREYYADSVKG

SEQ ID NO: 412 - protein, 9 aa, Artificial Sequence
  Synthetic
GSEGDGFDV

SEQ ID NO: 413 - protein, 11 aa, Artificial Sequence
  Synthetic
RASQRISSYLN

SEQ ID NO: 414 - protein, 7 aa, Artificial Sequence
  Synthetic
AASSLQS

SEQ ID NO: 415 - protein, 9 aa, Artificial Sequence
  Synthetic
QQSYSAPYT

SEQ ID NO: 416 - protein, 5 aa, Artificial Sequence
  Synthetic
DYGMH

SEQ ID NO: 417 - protein, 17 aa, Artificial Sequence
  Synthetic
VISYEGSNQYYADSVKG

SEQ ID NO: 418 - protein, 9 aa, Artificial Sequence
  Synthetic
GSEGDGFDV

SEQ ID NO: 419 - protein, 11 aa, Artificial Sequence
  Synthetic
RASQRIGNYLN

SEQ ID NO: 420 - protein, 7 aa, Artificial Sequence
  Synthetic
AASSLQS

SEQ ID NO: 421 - protein, 9 aa, Artificial Sequence
  Synthetic
QQGYSPPYT

SEQ ID NO: 422 - protein, 5 aa, Artificial Sequence
  Synthetic
DYGMH

SEQ ID NO: 423 - protein, 17 aa, Artificial Sequence
  Synthetic
VISYEGSREYYADSVKG

SEQ ID NO: 424 - protein, 9 aa, Artificial Sequence
  Synthetic
GSEGDGFDV

SEQ ID NO: 425 - protein, 11 aa, Artificial Sequence
  Synthetic
RASQRISNYLN

SEQ ID NO: 426 - protein, 7 aa, Artificial Sequence
  Synthetic
AASSLQS

SEQ ID NO: 427 - protein, 9 aa, Artificial Sequence
  Synthetic
QQGYSPPYT

SEQ ID NO: 428 - protein, 5 aa, Artificial Sequence
  Synthetic
DYGMH

SEQ ID NO: 429 - protein, 17 aa, Artificial Sequence
  Synthetic
VISYEGSREYYADSVKG

SEQ ID NO: 430 - protein, 9 aa, Artificial Sequence
  Synthetic
GSEGDGFDV

SEQ ID NO: 431 - protein, 11 aa, Artificial Sequence
  Synthetic
RASQRISKYLN

SEQ ID NO: 432 - protein, 7 aa, Artificial Sequence
  Synthetic
AASSLQS

SEQ ID NO: 433 - protein, 9 aa, Artificial Sequence
  Synthetic
QQSYSPPYT

SEQ ID NO: 434 - protein, 5 aa, Artificial Sequence
  Synthetic
DYGMH

SEQ ID NO: 435 - protein, 17 aa, Artificial Sequence
  Synthetic
VISYEGSREYYADSVKG

SEQ ID NO: 436 - protein, 9 aa, Artificial Sequence
  Synthetic
GSEGDGFDV

SEQ ID NO: 437 - protein, 11 aa, Artificial Sequence
  Synthetic
RASQRIYNYLN

SEQ ID NO: 438 - protein, 7 aa, Artificial Sequence
  Synthetic
AASSLQS

SEQ ID NO: 439 - protein, 9 aa, Artificial Sequence
  Synthetic
QQSYSPPYT

SEQ ID NO: 440 - protein, 5 aa, Artificial Sequence
  Synthetic
DYGMH

SEQ ID NO: 441 - protein, 17 aa, Artificial Sequence
  Synthetic
VISYEGSREYYADSVKG

SEQ ID NO: 442 - protein, 9 aa, Artificial Sequence
  Synthetic
GSEGDGFDV

SEQ ID NO: 443 - protein, 11 aa, Artificial Sequence
  Synthetic
RASQTISNYLN

SEQ ID NO: 444 - protein, 7 aa, Artificial Sequence
  Synthetic
AASSLQS

SEQ ID NO: 445 - protein, 9 aa, Artificial Sequence
  Synthetic
QQSYSPPYT

SEQ ID NO: 446 - protein, 5 aa, Artificial Sequence
  Synthetic
NYAMS

SEQ ID NO: 447 - protein, 17 aa, Artificial Sequence
  Synthetic
AISGGGSYTYYADSVKG

SEQ ID NO: 448 - protein, 11 aa, Artificial Sequence
  Synthetic
EERVLGYSLDY

SEQ ID NO: 449 - protein, 11 aa, Artificial Sequence
  Synthetic
RASQRIYKYLN

SEQ ID NO: 450 - protein, 7 aa, Artificial Sequence
  Synthetic
AASSLQS

SEQ ID NO: 451 - protein, 9 aa, Artificial Sequence
  Synthetic
QQSFSYPLT

(19) United States
(12) Patent Application Publication
(10) Pub. No.: US 2025/0333497 A1
(43) Pub. Date: Oct. 30, 2025

(54) ACTIVATING ANTIBODIES OF RECEPTOR PROTEINS PLXDC1 AND PLXDC2

(71) Applicants: THE REGENTS OF THE UNIVERSITY OF CALIFORNIA, Oakland, CA (US); ATENGEN, INC., Los Angeles, CA (US)

(72) Inventors: Hui SUN, Los Angeles, CA (US); Adrian Chichuen AU, Los Angeles, CA (US); Guo CHENG, Los Angeles, CA (US); Pu SUN, Los Angeles, CA (US)

(73) Assignees: THE REGENTS OF THE UNIVERSITY OF CALIFORNIA, Oakland, CA (US); ATENGEN, INC., Los Angeles, CA (US)

(21) Appl. No.: 18/857,970

(22) PCT Filed: Apr. 19, 2022
(86) PCT No.: PCT/US2022/025355

§ 371 Date: Oct. 18, 2024

(51) Int. Cl. C07K 16/28; A61P 35/00

(52) U.S. Cl. CPC C07K 16/28; A61P 35/00; C07K 2317/31; C07K 2317/33; C07K 2317/565; C07K 2317/75; C07K 2317/76

(57) ABSTRACT

PLXDC1 and PLXDC2 represent a new cell-surface receptor family (collectively referred to as PLXDC proteins). The present disclosure reports that Domain A of the PLXDC proteins functions as an inhibitory domain, as the deletion thereof activates PLXDC signaling. Antibodies and antigen-binding fragments that bind to Domain A can therefore relieve its inhibitory function and activate PLXDC signaling, leading to killing of endothelial cells in pathogenic blood vessel that express the PLXDC protein. Methods are described for efficient screening of PLXDC-activating antibodies that bind to Domain A. In particular, the method entails the use of a small molecule agent that binds the PLXDC protein, making Domain A more accessible to a test antibody. With the new method, antibodies that bind to Domain A and can activate PLXDC signaling have been successfully identified. These antibodies, as well as their antigen-binding fragments, are useful for treating diseases characterized with PLXDC-expressing pathogenic blood vessels.

### BACKGROUND

Angiogenesis plays a key role in the pathogenesis of some major human diseases. In addition to tumor growth and metastasis, angiogenesis is a major driving force in several blinding diseases including diabetic retinopathy, age-related macular degeneration (AMD), and retinopathy of prematurity. AMD and diabetic retinopathy are the leading causes of blindness in the elderly and populations at the working age in the United States, respectively. Retinopathy of prematurity is a common reason that causes the loss of vision for newborn babies.

Angiogenesis also plays a role in pathogenesis of cancer, e.g., tumor development, since newly-formed blood vessels supply the tumor with growth nutrients and signals that allow the tumor to grow and spread. Accordingly, cutting off a tumor's supply of nutrients and primary mechanism for traveling to distant sites is an attractive therapeutic strategy. However, current anti-angiogenic strategies only target newly formed blood vessels, and are unable to target existing blood vessels that contribute to disease progression.

Different disease progression patterns can be induced by anti-angiogenic therapies, which may lead to worse outcomes in terms of drug resistance, invasion, and metastasis. Furthermore, targeting angiogenesis does not treat existing blood vessels that may have, for example, already vascularized a tumor. There is a need in the art for complementary therapies that, in contrast to anti-angiogenic therapies, can target existing blood vessels and treat cancer and other disorders arising from angiogenesis (collectively referred to herein as pathogenic blood vessel disorders).

### SUMMARY

The current disclosure provides an advancement over the conventional anti-angiogenic strategies that target the generation of new blood vessels, by providing compositions and methods that also can selectively target and kill existing pathogenic blood vessels.

PLXDC1 and PLXDC2 represent a new cell-surface receptor family. They are hereby referred to as the PLXDC family of receptors, or simply PLXDC or PLXDC proteins. The present disclosure reports that Domain A of the PLXDC proteins functions as an inhibitory domain, as the deletion of Domain A activates PLXDC signaling. Antibodies and antigen-binding fragments that bind to Domain A can therefore inhibit its inhibitory function and activate PLXDC signaling, leading to killing of pathogenic blood vessel cells that express the PLXDC protein. Methods are described for efficient screening of PLXDC-activating antibodies that bind to Domain A. In particular, the method entails the use of a small molecule agent that binds the PLXDC protein, making Domain A more accessible to a test antibody. With the new method, antibodies that bind to Domain A and can activate PLXDC signaling have been successfully identified. These antibodies, as well as their antigen-binding fragments, are useful for treating diseases characterized with PLXDC-expressing pathogenic blood vessels.

In accordance with one embodiment of the present disclosure, provided is a method for inhibiting the growth of or killing cells in a pathogenic blood vessel in a patient in need thereof, comprising administering to the patient an antibody or antigen-binding fragment thereof that binds Domain A of a plexin domain-containing (PLXDC) protein. In some embodiments, the antibody or antigen-binding fragment thereof inhibits dimerization of the Domain A. In some embodiments, the antibody or antigen-binding fragment thereof binds to at least an amino acid residue involved in Domain A dimerization.

In some embodiments, the antibody or antigen-binding fragment thereof does not bind any one of Domains B-E of the PLXDC protein. In some embodiments, the antibody or antigen-binding fragment thereof activates PLXDC signaling upon binding to the PLXDC protein.

In some embodiments, the antibody or antigen binding fragment thereof binds to the PLXDC protein with a higher affinity in the presence of a small molecule compound that binds and activates the PLXDC protein, as compared to when the small molecule compound is not present. In some embodiments, the antibody or antigen binding fragment thereof is not capable of mediating antibody-dependent cell-mediated cytotoxicity (ADCC).

In some embodiments, the antibody is a bispecific antibody that further has a second specificity to an immune cell. In some embodiments, the PLXDC protein is PLXDC1 or PLXDC2.

In some embodiments, the antibody or antigen binding fragment thereof is an antibody selected from Table 4 or an antigen binding fragment thereof, is an antibody or antigen binding fragment thereof that includes the complementarity-determining regions (CDR) VH CDR1, VH CDR2, VH CDR3, VL CDR1, VL CDR2 and VL CDR3 of an antibody selected from Table 4, or is an antibody or antigen binding fragment thereof that competes with an antibody selected from Table 4 in binding to PLXDC1. In some embodiments, the antibody or antigen binding fragment thereof inhibits dimerization of the Domain A and does not include all of the CDRs of any one of antibodies A001 to A010.

In some embodiments, the patient has a disorder selected from the group consisting of diabetic retinopathy, age-related macular degeneration (AMD), retinopathy of prematurity, cancer and combinations thereof.

Also provided, in one embodiment, is a recombinant antibody or antigen-binding fragment thereof that binds Domain A of a plexin domain-containing (PLXDC) protein. In some embodiments, the recombinant antibody or antigen-binding fragment inhibits dimerization of the Domain A. In some embodiments, the recombinant antibody or antigen-binding fragment binds to at least an amino acid residue involved in Domain A dimerization.

In some embodiments, the recombinant antibody or antigen-binding fragment does not bind any one of Domains B-E of the PLXDC protein. In some embodiments, the recombinant antibody or antigen-binding fragment activates PLXDC signaling upon binding to the PLXDC protein.

In some embodiments, the recombinant antibody or antigen-binding fragment binds to the PLXDC protein with a higher affinity in the presence of a small molecule compound that binds and activates the PLXDC protein, as compared to when the small molecule compound is not present. In some embodiments, the recombinant antibody or antigen-binding fragment is not capable of mediating antibody-dependent cell-mediated cytotoxicity (ADCC). In some embodiments, the antibody is a bispecific antibody that further has a second specificity to an immune cell.

In some embodiments, the PLXDC protein is PLXDC1 or PLXDC2. In some embodiments, the recombinant antibody or antigen-binding fragment is an antibody selected from Table 4 or an antigen binding fragment thereof, is an antibody or antigen binding fragment thereof that includes the complementarity-determining regions (CDR) VH CDR1, VH CDR2, VH CDR3, VL CDR1, VL CDR2 and VL CDR3 of an antibody selected from Table 4, or is an antibody or antigen binding fragment thereof that competes with an antibody selected from Table 4 in binding to PLXDC1. In some embodiments, the recombinant antibody or antigen-binding fragment inhibits dimerization of the Domain A and does not include all of the CDRs of any one of antibodies A001 to A010.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrates the domain structure, and the role of Domain A in activating the PLXDC protein. FIG. 1A is a schematic diagram of the domain structure of PLXDC protein, which is a single transmembrane domain protein that consists of extracellular Domains A, B, C, D, the transmembrane domain (TM), and an intracellular domain. Domain A is an inhibitory domain and deletion of domain A leads to the activation of the receptor. FIG. 1B is a schematic diagram showing the binding of antibody specific for domain A can also relieve the inhibition by domain A and lead to receptor activation.

FIG. 2 shows that expression of Domain A-deleted PLXDC1 led to death of adherent cells. PLXDC-mediated cell death pathway is similar to anoikis and only applies to adherent cells such as endothelial cells, not to nonadherent cells. This experiments shows that expression of domain A-deleted PLXDC1 (PLXDC1-dA), but not full-length PLXDC1 (PLXDC1), led to cell death in an adherent cell model. In contrast, both domain A-deleted PLXDC1 (PLXDC1-dA) and full-length PLXDC1 (PLXDC1) do not cause cell death in a nonadherent cell model.

FIG. 3 shows that antibodies A001-A003 specifically bound to Domain A of PLXDC1 and not any other domains.

FIG. 4 shows that antibodies A004-A007 specifically bound to Domain A of PLXDC1 and not any other domains.

FIG. 5 shows that addition of a small molecule agonist greatly enhanced the binding of the activating antibodies to PLXDC1.

### DETAILED DESCRIPTION

### I. Definitions

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

As used herein, the terms “treat” or “treatment” refer to both therapeutic treatment and prophylactic or preventative measures, wherein the object is to prevent or slow down (lessen) an undesired physiological change or disorder, such as the progression of cancer or of a pathogenic blood vessel disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (X) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COH end. The isotype of an antibody can be IgM, IgD, IgG, IgA, or IgE and is defined by the heavy chains present of which there are five classifications: mu (φ), delta (d), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Bispecific antibodies can be constructed as: a whole IgG, Fab′2, Fab′PEG, a diabody, or alternatively as scFv.

Certain aspects relate to antibody fragments, such as antibody fragments that bind to PLXDC. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20- fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

### II. Activating Antibodies of PLXDC

Tumor development and survival rely on vascularization for the supply of growth factors and nutrients and as a mechanism for metastasizing to distant sites. Conventional antiangiogenic drugs inhibit angiogenesis and hence the growth of the tumor, but cannot kill the tumor because they cannot effectively kill existing tumor blood vessels. The instant inventors conducted research into the plexin domain-containing (PLXDC) proteins (e.g., PLXDC1 and PLXDC2) that are expressed in pathogenic blood vessels. Activation of the plexin domain-containing proteins (PLXDC) leads to effective killing of the endothelial cells, thereby providing a novel modality for eliminating or reducing a primary mechanism of pathogenicity.

The term “plexin domain-containing protein” or “PLXDC” refers to a small transmembrane receptor family that includes PLXDC1 and PLXDC2. This family of proteins have a large extracellular portion, a transmembrane domain, and share high sequence homology. Without limitation, the PLXDC protein can be PLXDC 1 or PLXDC2. PLXDC 1 has a protein sequence as shown in NCBI Reference Sequence: NP_065138.2 (SEQ ID NO:1), PLXDC2 has a sequence of NCBI Reference Sequence: NP_116201.7 (SEQ ID NO:2, isoform 1) or NP_001269665 (SEQ ID NO:3, isoform 2). Their sequences and estimated domain structures are shown in Tables 1 and 2 below.

TABLE 1
PLXDC sequences, and alignment and estimated domain structures

PLXDC1 (SEQ ID NO: 1):
MRGELWLLVLVLREAARALSPQPGAGHDEGPGSGWAAKGTVRGWNRRARESPGHVSEPDRTQLSQDLGGGTLA
MDTLPDNRTRVVEDNHSYYVSRLYGPSEPHSRELWVDVAEANRSQVKIHTILSNTHRQASRVVLSFDFPFYGH
PLRQITIATGGFIFMGDVIHRMLTATQYVAPLMANFNPGYSDNSTVVYFDNGTVFVVQWDHVYLQGWEDKGSF
TFQAALHHDGRIVFAYKEIPMSVPEISSSQHPVKTGLSDAFMILNPSPDVPESRRRSIFEYHRIELDPSKVTS
MSAVEFTPLPTCLQHRSCDACMSSDLTFNCSWCHVLQRCSSGFDRYRQEWMDYGCAQEAEGRMCEDFQDEDHD
SASPDTSFSPYDGDLTTTSSSLFIDSLTTEDDTKLNPYAGGDGLQNNLSPKTKGTPVHLGTIVGIVLAVLLVA
AIILAGIYINGHPTSNAALFFIERRPHHWPAMKFRSHPDHSTYAEVEPSGHEKEGFMEAEQC
PLXDC2, isoform 1 (SEQ ID NO: 2):
MARFPKADLAAAGVMLLCHFFTDQFQFADGKPGDQILDWQYGVTQAFPHTEEEVEVDSHAYSHRWKRNLDELK
AVDTNRASVGQDSPEPRSFTDLLLDDGQDNNTQIEEDTDHNYYISRIYGPSDSASRDLWVNIDQMEKDKVKIH
GILSNTHRQAARVNLSFDFPFYGHFLREITVATGGFIYTGEVVHRMLTATQYIAPLMANFDPSVSRNSTVRYF
DNGTALVVQWDHVHLQDNYNLGSFTFQATLLMDGRIIFGYKEIPVLVTQISSTNHPVKVGLSDAFVVVHRIQQ
IPNVRRRTIYEYHRVELQMSKITNISAVEMTPLPTCLQFNRCGPCVSSQIGFNCSWCSKLQRCSSGFDRHRQD
WVDSGCPEESKEKMCENTEPVETSSRTTTTVGATTTQFRVLTTTRRAVTSQFPTSLPTEDDTKIALHLKDNGA
STDDSAAEKKGGTLHAGLIIGILILVLIVATAILVTVYMYHHPTSAASIFFIERRPSRWPAMKFRRGSGHPAY
AEVEPVGEKEGFIVSEQC
PLXDC2, isoform 2 (SEQ ID NO: 3):
MARFPKADLAAAGVMLLCHFFTDQFQFADGKPGDQILDWQYGVTQAFPHTEEEVEVDSHAYSHRWKRNLDELK
AVDTNRASVGQDSPEPRSFTDLLLDDGQDNNTQIERVNLSFDFPFYGHFLREITVATGGFIYTGEVVHRMLTA
TQYIAPLMANFDPSVSRNSTVRYFDNGTALVVQWDHVHLQDNYNLGSFTFQATLLMDGRIIFGYKEIPVLVTQ
ISSTNHPVKVGLSDAFVVVHRIQQIPNVRRRTIYEYHRVELQMSKITNISAVEMTPLPTCLQFNRCGPCVSSQ
IGFNCSWCSKLQRCSSGFDRHRQDWVDSGCPEESKEKMCENTEPVETSSRTTTTVGATTTQFRVLTTTRRAVT
SQFPTSLPTEDDTKIALHLKDNGASTDDSAAEKKGGTLHAGLIIGILILVLIVATAILVTVYMYHHPTSAASI
FFIERRPSRWPAMKFRRGSGHPAYAEVEPVGEKEGFIVSEQC
Alignment (SEQ ID NOS: 1-3, respectively, are aligned below):

| | | |
|---|---|---|
| PLXDC1 | --------MRGELWLLVLVLREAARALSPQPGAGHDEGPGSGWA-------------- | 36 |
| PLXDC2_1 | MARFPKADLAAAGVMLLCHFFTDQFQFADGKPGD----QILDWQYGVTQAFPHTEEEVE | 55 |
| PLXDC2_2 | MARFPKADLAAAGVMLLCHFFTDQFQFADGKPGD----QILDWQYGVTQAFPHTEEEVE | 55 |
| | ________________________________ | |
| | Domain A | |
| PLXDC1 | --AKGTVRGWNRRARESPGHVSEPDRTQLSQDLG----GGTLAMDTLPDNRTR-VVEDNH | 89 |
| PLXDC2_1 | VDSHAYSHRWKRNLDELK--AVDTNRASVGQDSPEPRSFTDLLLDDGQDNNTQIEEDTDH | 113 |
| PLXDC2_2 | VDSHAYSHRWKRNLDELK--AVDTNRASVGQDSPEPRSFTDLLLDDGQDNNTQIE----- | 108 |
| | ____________________________________________________________ | |
| | Domain A | |
| PLXDC1 | SYYVSRLYGPSEPHSRELWVDVAEANRSQVKIHTILSNTHRQASRVVLSFDFPFYGHPLR | 149 |
| PLXDC2_1 | NYYISRIYGPSDSASRDLWVNIDQMEKDKVKIHGILSNTHRQAARVNLSFDFPFYGHFLR | 173 |
| PLXDC2_2 | --------------------------------------------RVNLSFDFPFYGHFLR | 124 |
| | ______________________________________====================== | |
| | Domain A Domain B | |
| PLXDC1 | QITIATGGFIFMGDVIHRMLTATQYVAPLMANFNPGYSDNSTVVYFDNGTVFVVQWDHVY | 209 |
| PLXDC2_1 | EITVATGGFIYTGEVVHRMLTATQYIAPLMANFDPSVSRNSTVRYFDNGTALVVQWDHVH | 233 |
| PLXDC2_2 | EITVATGGFIYTGEVVHRMLTATQYIAPLMANFDPSVSRNSTVRYFDNGTALVVQWDHVH | 184 |
| | ============================================================ | |
| | Domain B | |
| PLXDC1 | LQGWEDKGSFTFQAALHHDGRIVFAYKEIPMSVPEISSSQHPVKTGLSDAFMILNPSPDV | 269 |
| PLXDC2_1 | LQDNYNLGSFTFQATLLMDGRIIFGYKEIPVLVTQISSTNHPVKVGLSDAFVVVHRIQQI | 293 |
| PLXDC2_2 | LQDNYNLGSFTFQATLLMDGRIIFGYKEIPVLVTQISSTNHPVKVGLSDAFVVVHRIQQI | 244 |
| | =================================\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ } | |
| | Domain B Domain C | |
| PLXDC1 | PESRRRSIFEYHRIELDPSKVTSMSAVEFTPLPTCLQHRSCDACMSSDLTFNCSWCHVLQ | 329 |
| PLXDC2_1 | PNVRRRTIYEYHRVELQMSKITNISAVEMTPLPTCLQFNRCGPCVSSQIGENCSWCSKLQ | 353 |
| PLXDC2_2 | PNVRRRTIYEYHRVELQMSKITNISAVEMTPLPTCLQFNRCGPCVSSQIGENCSWCSKLQ | 304 |
| | \hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }~~~~~~~~~~~~~~~~~~~~~~~~~~~~~~~~~~~~~ | |
| | Domain C Domain D | |
| PLXDC1 | RCSSGFDRYRQEWMDYGCAQEAEGRMCEDFQDEDHDSASPDT------SFSPYDGDLITT | 383 |
| PLXDC2_1 | RCSSGFDRHRQDWVDSGCPEESKEKMCENTEPVETSSRTTTTVGATTTQFRVLT-TTRRA | 412 |
| PLXDC2_2 | RCSSGFDRHRQDWVDSGCPEESKEKMCENTEPVETSSRTTTTVGATTTQFRVLT-TTRRA | 363 |
| | ~~~~~~~~~~~~~~~~~~~~~~~~~*********************************** | |
| | Domain D Domain E | |
| PLXDC1 | SSSLFIDSLTTEDDTKLNPYAGGDGLQ-NNLSPKTKGTPVHLGTIVGIVLAVLLVAAIIL | 442 |
| PLXDC2_1 | VTSQFPTSLPTEDDTKIALHLKDNGASTDDSAAEKKGGTLHAGLIIGILILVLIVATAIL | 472 |
| PLXDC2_2 | VTSQFPTSLPTEDDTKIALHLKDNGASTDDSAAEKKGGTLHAGLIIGILILVLIVATAIL | 423 |
| | ********************************************================ | |
| | Domain E Transmembrane | |
| PLXDC1 | AGIYINGHPTSNAALFFIERRPHHWPAMKFRSHPDHSTYAEVEPSGHEKEGFMEAEQC | 500 |
| PLXDC2_1 | VTVYMYHHPTSAASIFFIERRPSRWPAMKFRRGSGHPAYAEVEPVG-EKEGFIVSEQC | 529 |
| PLXDC2_2 | VTVYMYHHPTSAASIFFIERRPSRWPAMKFRRGSGHPAYAEVEPVG-EKEGFIVSEQC | 480 |
| | =====_____________________________________________________ | |
| | Intracellular | |

TABLE 2
Listing of domains and locations

| Protein (SEQ ID NO:) | Domain | Amino acid residues |
|---|---|---|
| PLXDC1 (SEQ ID NO: 1) | Domain A | 19-127 |
| | Domain B | 128-242 |
| | Domain C | 243-292 |
| | Domain D | 293-359 |
| | Domain E | 360-427 |
| PLXDC2 isoform 1 (SEQ ID NO: 2) | Domain A | 31-151 |
| | Domain B | 152-266 |
| | Domain C | 267-316 |
| | Domain D | 317-383 |
| | Domain E | 384-454 |
| PLXDC2 isoform 2 (SEQ ID NO: 3) | Domain A | 31-108 |
| | Domain B | 109-207 |
| | Domain C | 208-267 |
| | Domain D | 268-334 |
| | Domain E | 335-405 |

PLXDC 1 and PLXDC2 are highly specifically expressed in the tumor blood vessels of diverse types of cancer, and in the pathogenic blood vessels in diabetic retinopathy. This high enrichment is not present in healthy blood vessels. High PLXDC1 expression has also been identified in choroidal neovascularization (pathogenic angiogenesis in age-related macular degeneration (AMD) and ischemia-induced retinopathy (pathogenic angiogenesis in retinopathy of prematurity). Killing cells that express PLXDC with an activating antibody, therefore, can effectively treat the related diseases.

Identification of an activating antibody, however, has tremendous challenges. A neutralizing antibody inhibits ligand/receptor interaction, such as Humira (inhibiting TNF-α, a ligand), Avastin (inhibiting VEGF, a ligand), Herceptin (inhibiting HER2, a receptor), and Keytruda (inhibiting PD-1, a receptor). A targeting antibody, on the other hand, may exert its function through mechanisms such as antibody-drug conjugates and antibody-dependent cell-mediated cytotoxicity (ADCC). No activating antibodies have been identified, in particular against single transmembrane cell-surface receptors like PLXDC1/PLXDC2.

Targeting antibodies of PLXDC have been developed as a potential anti-angiogenic therapy. In Bagley et al., Microvasc Res. 2011 November; 82(3):253-62, an anti-PLXDC1 antibody was identified that mediated antibody-dependent cellular cytotoxicity (ADCC) and phagocytosis. Such cancer immunotherapy approaches, however, have not yielded positive therapeutic results. There is a need, therefore, to identify true PLXDC-activating antibodies.

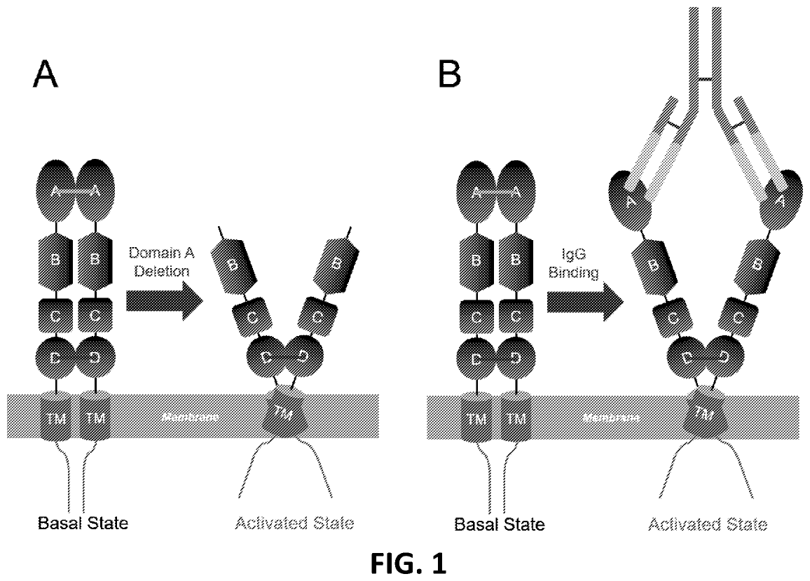

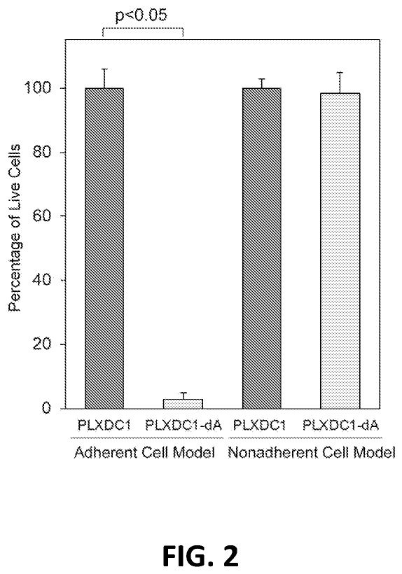

The instant inventors made an unexpected discovery that Domain A of the PLXDC functions as an inhibitory domain. Deletion of Domain A led to receptor activation and triggered the downstream cell death pathway (FIG. 1, right panel, and FIG. 2). It is contemplated that Domain A exerts the inhibitory function by binding to the Domain A of another PLXDC protein (FIG. 1, left panel), thereby keeping both copies of the PLXDC protein in a basal/dormant state. An antibody that interferes with or inhibits Domain A dimerization, therefore, can effectively block the inhibitory function of Domain A, leading to activation of the PLXDC protein.

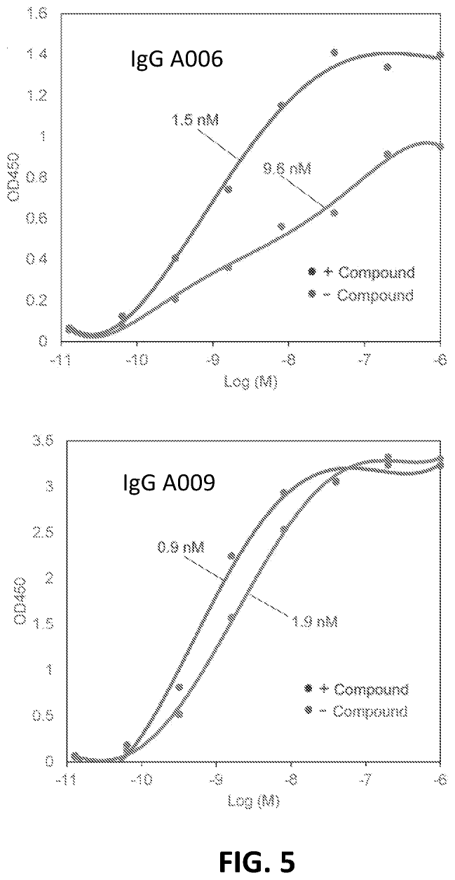

It was also observed, however, that screening for Domain A-inhibiting antibodies is highly challenging as well, likely because PLXDC proteins are present in dimerized forms, which blocks entry of the test antibodies. In this context, the instant inventors have designed a greatly improved antibody screening assay. In an example assay, the screening is conducted in the presence of a small molecule PLXDC agonist agent, which can promote the activated conformation of the receptor. Screening for antibodies that preferentially bind to the activated conformation can lead to the identification of agonist antibodies. The small molecule agent binds to the PLXDC protein and dissociates dimerized Domain A's, making Domain A accessible to the test antibody. As demonstrated in Example 5 (FIG. 5), when the small molecule agonist is present, the Domain A-binding antibodies exhibited greatly higher affinity to the PLXDC protein. In a preferred embodiment, the small molecule PLXDC-binding agent does not bind to Domain A, and nevertheless is capable of interrupting or loosening up Domain A dimerization. An example of the small molecule PLXDC-binding agent is Compound 369, with the structure shown below. The preparation and use of Compound 369 are described in PCT Patent Application WO 2021/076930, the content of which is incorporated to the present disclosure by reference.

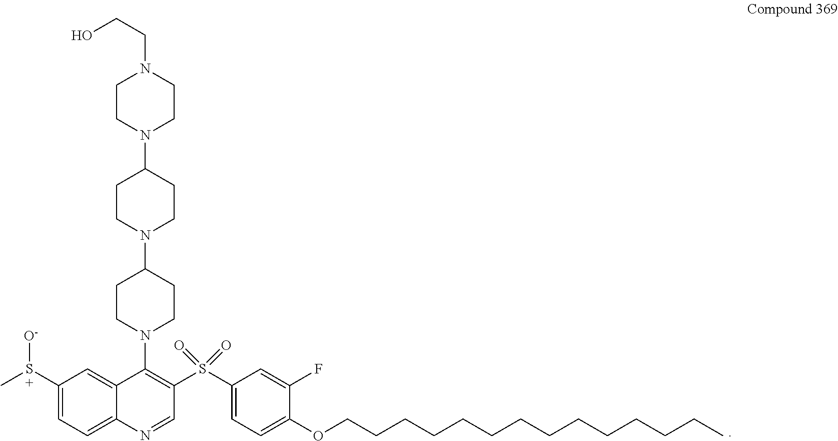

With such a greatly improved screening assay, the inventors were able to quickly evaluate antibodies from a large custom human antibody library. Thirty-one clones (Table 1) were confirmed to have potent affinity to PLXDC Domain A and can effectively activate PLXDC signaling, leading to blood vessel cell killing.

In accordance with one embodiment of the present disclosure, provided is an antibody or antigen-binding fragment thereof that binds Domain A of a plexin domain-containing (PLXDC) protein. The antibody or fragment may be an isolated antibody or fragment, or a recombinant antibody or fragment, to be distinguishable from naturally occurring ones.

As provided in Table 2, Domain A of PLXDC1 includes amino acids 19-127 of SEQ ID NO:1, Domain A of PLXDC2 isoform 1 includes amino acids 31-151 of SEQ ID NO:2, and Domain A of PLXDC2 isoform 2 includes amino acids 31-108 of SEQ ID NO:3.

In some embodiments, the antibody or antigen-binding fragment thereof inhibits dimerization of the Domain A. Domain A dimerization, it is contemplated, requires amino acid residues involved in binding between two copies of the domain. Certain other amino acid residues, whether or not within Domain A, may also impact the dimerization as they may be important to maintain the proper three-dimensional structure of the domain or PLXDC protein which is required for Domain A dimerization.

Nevertheless, in a preferred embodiment, the antibody or antigen-binding fragment thereof binds to at least one amino acid involved in Domain A-Domain A binding. In some embodiments, the antibody or antigen-binding fragment thereof binds to at least 2, or at least 3, 4, 5, 6, 7, or 8 amino acids involved in Domain A-Domain A binding.

In some embodiments, the antibody or antigen-binding fragment thereof binds amino acid residue 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, and/or 127 of PLXDC1 (SEQ ID NO:1). In some embodiments, the antibody or antigen-binding fragment thereof binds amino acid residue 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 and/or 151 of PLXDC2 isoform 1 (SEQ ID NO:2). In some embodiments, the antibody or antigen-binding fragment thereof binds amino acid residue 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107 and/or 108 of PLXDC2 isoform 2 (SEQ ID NO:3).

In some embodiments, the antibody or antigen-binding fragment thereof binds to the monomeric form of PLXDC with greater affinity than the dimeric form. For example, the K_{D} for the monomeric form may be less than about 10⁻⁴, 10⁻⁵, 10⁻⁶, 10⁻⁷, 10⁻⁸, 10⁻⁹, 10⁻¹⁰, 10⁻¹¹, 10⁻¹², 10¹³, 10⁻¹⁴, or 10⁻¹⁵ M (or any derivable range therein), and the K_{D} for the dimeric form may be greater than about 10⁻¹, 10⁻², 10⁻³, 10⁻⁴, 10⁻⁵, or 10⁻⁶ M (or any derivable range therein).

In some embodiments, the antibody or antigen-binding fragment thereof does not bind to another domain of the PLXDC protein. In some embodiments, the antibody or antigen-binding fragment thereof does not bind to Domain B of the PLXDC protein. In some embodiments, the antibody or antigen-binding fragment thereof does not bind to Domain C of the PLXDC protein. In some embodiments, the antibody or antigen-binding fragment thereof does not bind to Domain D of the PLXDC protein. In some embodiments, the antibody or antigen-binding fragment thereof does not bind to Domain E of the PLXDC protein. In some embodiments, the antibody or antigen-binding fragment thereof does not bind to any one of Domains B-E of the PLXDC protein.

The specific binding to Domain A of a PLXDC protein can be tested with a method described herein. In some embodiments, the method employs a PLXDC with the Domain A deleted. Binding to the wild-type PLXDC along with not binding to the truncated form indicates that the antibody or fragment specifically binds to Domain A. In some embodiments, the binding assay is conducted in the presence of a small molecule agent that binds PLXDC, making Domain A more accessible to the antibody. An example is Compound 369 as described above.

In some embodiments, the antibody or antigen-binding fragment thereof is capable of activating PLXDC signaling upon binding to the PLXDC protein. In some embodiments, the binding is able to cause activation of one or more genes in the tumor endothelial cell, which may be regulated by PLXDC, leading to tumor blood vessel death and tumor necrosis.

Non-limiting examples of such genes include ADAM17 (ADAM metallopeptidase domain 17), BAG4 (BCL2 associated athanogene 4), BIRC2 (baculoviral IAP repeat containing 2), BIRC3 (baculoviral IAP repeat containing 3), CASP8 (caspase 8), CAV1 (caveolin 1), CHUK (component of inhibitor of nuclear factor kappa B kinase complex), CYLD (CYLD lysine 63 deubiquitinase), FADD (Fas associated via death domain), IKBKB (inhibitor of nuclear factor kappa B kinase subunit beta), IKBKG (inhibitor of nuclear factor kappa B kinase regulatory subunit gamma), ITCH (itchy E3 ubiquitin protein ligase), MADD (MAP kinase activating death domain), MAP2K3 (mitogen-activated protein kinase kinase 3), MAP2K7 (mitogen-activated protein kinase kinase 7), MAP3K1 (mitogen-activated protein kinase kinase kinase 1), MAP3K3 (mitogen-activated protein kinase kinase kinase 3), MAP3K5 (mitogen-activated protein kinase kinase kinase 5), MAP3K7 (mitogen-activated protein kinase kinase kinase 7), MAP4K2 (mitogen-activated protein kinase kinase kinase kinase 2), MAP4K3 (mitogen-activated protein kinase kinase kinase kinase 3), MAP4K4 (mitogen-activated protein kinase kinase kinase kinase 4), MAP4K5 (mitogen-activated protein kinase kinase kinase kinase 5), NFKB1 (nuclear factor kappa B subunit 1), NRK (Nik related kinase), NSMAF (neutral sphingomyelinase activation associated factor), PRKCI (protein kinase C iota), PRKCZ (protein kinase C zeta), RACK1 (receptor for activated C kinase 1), RBCK1 (RANBP2-type and C3HC4-type zinc finger containing 1), RELA (RELA proto-oncogene, NF-κB subunit), RFFL (ring finger and FYVE like domain containing E3 ubiquitin protein ligase), RIPK1 (receptor interacting serine/threonine kinase 1), RNF11 (ring finger protein 11), RNF31 (ring finger protein 31), SHARPIN (SHANK associated RH domain interactor), SMPD1 (sphingomyelin phosphodiesterase 1), SMPD2 (sphingomyelin phosphodiesterase 2), SQSTM1 (sequestosome 1), STAT1 (signal transducer and activator of transcription 1), TAB1 (TGF-beta activated kinase 1 (MAP3K7) binding protein 1), TAB2 (TGF-beta activated kinase 1 (MAP3K7) binding protein 2), TAB3 (TGF-beta activated kinase 1 (MAP3K7) binding protein 3), TAX1BP1 (Taxi binding protein 1), TNF (tumor necrosis factor), TNFAIP3 (TNF alpha induced protein 3), TNFRSF1A (TNF receptor superfamily member 1A), TNFRSF1B (TNF receptor superfamily member 1B), TNIK (TRAF2 and NCK interacting kinase), TRADD (TNFRSF1A associated via death domain), TRAF1 (TNF receptor associated factor 1), TRAF2 (TNF receptor associated factor 2), TRAF5 (TNF receptor associated factor 5), and TXN (thioredoxin).

In some embodiments, the antibody or antigen binding fragment thereof is capable of mediating antibody-dependent cell-mediated cytotoxicity (ADCC), such as a suitable Fc fragment that is ADCC-competent. In some embodiments, the antibody or antigen binding fragment thereof is not capable of mediating ADCC.

Also provided are bispecific antibodies that incorporate the antibody or antigen binding fragment thereof disclosed herein. In some embodiments, the bispecific antibody further has a second specificity to an immune cell. Example antigens on an immune cell include CD3, CD16, CD19, CD28, CD64, PD-1, PD-L1, CTLA-4, and SIRPa.

### Example Domain A-Binding PLXDC-Activating Antibodies

Example antibodies that are capable of binding to Domain A and activating PLXDC1 are disclosed in the experimental examples (e.g., Table 4). In some embodiments, the antibody or antigen binding fragment thereof comprises a heavy chain variable region (VH) and a light chain variable region (VL), the VH comprises a VH complementarity-determining region (CDR) CDR1, a VH CDR2, a VH CDR3, the VL comprises a VL CDR1, a VL CDR2, and a VL CDR3. In some embodiments, the VH CDR1, VH CDR2, VH CDR3, VL CDR1, VL CDR2, and VL CDR3 are those of any antibody selected from Table 4, such as those provided in Table 5.

In one embodiment, the VH CDR1, VH CDR2, VH CDR3, VL CDR1, VL CDR2, and VL CDR3 are those of antibody A001. In one embodiment, the VH CDR1, VH CDR2, VH CDR3, VL CDR1, VL CDR2, and VL CDR3, respectively, include the amino acid sequences of SEQ ID NO:266-271. In one embodiment, the VH and VL of the antibody or antigen binding fragment thereof, respectively, include the amino acid sequences of SEQ ID NO:38-39, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A002. In one embodiment, the VH CDR1, VH CDR2, VH CDR3, VL CDR1, VL CDR2, and VL CDR3, respectively, include the amino acid sequences of SEQ ID NO:272-277. In one embodiment, the VH and VL of the antibody or antigen binding fragment thereof, respectively, include the amino acid sequences of SEQ ID NO:82-83, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A003. In one embodiment, the VH CDR1, VH CDR2, VH CDR3, VL CDR1, VL CDR2, and VL CDR3, respectively, include the amino acid sequences of SEQ ID NO:278-283. In one embodiment, the VH and VL of the antibody or antigen binding fragment thereof, respectively, include the amino acid sequences of SEQ ID NO:98-99, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A004. In one embodiment, the VH CDR1, VH CDR2, VH CDR3, VL CDR1, VL CDR2, and VL CDR3, respectively, include the amino acid sequences of SEQ ID NO:284-289. In one embodiment, the VH and VL of the antibody or antigen binding fragment thereof, respectively, include the amino acid sequences of SEQ ID NO:20-21, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A005. In one embodiment, the VH CDR1, VH CDR2, VH CDR3, VL CDR1, VL CDR2, and VL CDR3, respectively, include the amino acid sequences of SEQ ID NO:290-295. In one embodiment, the VH and VL of the antibody or antigen binding fragment thereof, respectively, include the amino acid sequences of SEQ ID NO:70-71, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A006. In one embodiment, the VH CDR1, VH CDR2, VH CDR3, VL CDR1, VL CDR2, and VL CDR3, respectively, include the amino acid sequences of SEQ ID NO:296-301. In one embodiment, the VH and VL of the antibody or antigen binding fragment thereof, respectively, include the amino acid sequences of SEQ ID NO:72-73, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A007. In one embodiment, the VH CDR1, VH CDR2, VH CDR3, VL CDR1, VL CDR2, and VL CDR3, respectively, include the amino acid sequences of SEQ ID NO:302-307. In one embodiment, the VH and VL of the antibody or antigen binding fragment thereof, respectively, include the amino acid sequences of SEQ ID NO:112-113, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A008. In one embodiment, the VH CDR1, VH CDR2, VH CDR3, VL CDR1, VL CDR2, and VL CDR3, respectively, include the amino acid sequences of SEQ ID NO:308-313. In one embodiment, the VH and VL of the antibody or antigen binding fragment thereof, respectively, include the amino acid sequences of SEQ ID NO:116-117, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A009. In one embodiment, the VH CDR1, VH CDR2, VH CDR3, VL CDR1, VL CDR2, and VL CDR3, respectively, include the amino acid sequences of SEQ ID NO:314-319. In one embodiment, the VH and VL of the antibody or antigen binding fragment thereof, respectively, include the amino acid sequences of SEQ ID NO:122-123, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A010. In one embodiment, the VH CDR1, VH CDR2, VH CDR3, VL CDR1, VL CDR2, and VL CDR3, respectively, include the amino acid sequences of SEQ ID NO:320-325. In one embodiment, the VH and VL of the antibody or antigen binding fragment thereof, respectively, include the amino acid sequences of SEQ ID NO:176-177, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A011. In one embodiment, the VH CDR1, VH CDR2, VH CDR3, VL CDR1, VL CDR2, and VL CDR3, respectively, include the amino acid sequences of SEQ ID NO:326-331. In one embodiment, the VH and VL of the antibody or antigen binding fragment thereof, respectively, include the amino acid sequences of SEQ ID NO:224-225, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A012. In one embodiment, the VH CDR1, VH CDR2, VH CDR3, VL CDR1, VL CDR2, and VL CDR3, respectively, include the amino acid sequences of SEQ ID NO:332-337. In one embodiment, the VH and VL of the antibody or antigen binding fragment thereof, respectively, include the amino acid sequences of SEQ ID NO:226-227, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A013. In one embodiment, the VH CDR1, VH CDR2, VH CDR3, VL CDR1, VL CDR2, and VL CDR3, respectively, include the amino acid sequences of SEQ ID NO:338-343. In one embodiment, the VH and VL of the antibody or antigen binding fragment thereof, respectively, include the amino acid sequences of SEQ ID NO:228-229, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A014. In one embodiment, the VH CDR1, VH CDR2, VH CDR3, VL CDR1, VL CDR2, and VL CDR3, respectively, include the amino acid sequences of SEQ ID NO:344-349. In one embodiment, the VH and VL of the antibody or antigen binding fragment thereof, respectively, include the amino acid sequences of SEQ ID NO:230-231, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A015. In one embodiment, the VH CDR1, VH CDR2, VH CDR3, VL CDR1, VL CDR2, and VL CDR3, respectively, include the amino acid sequences of SEQ ID NO:350-355. In one embodiment, the VH and VL of the antibody or antigen binding fragment thereof, respectively, include the amino acid sequences of SEQ ID NO:232-233, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A016. In one embodiment, the VH CDR1, VH CDR2, VH CDR3, VL CDR1, VL CDR2, and VL CDR3, respectively, include the amino acid sequences of SEQ ID NO:356-361. In one embodiment, the VH and VL of the antibody or antigen binding fragment thereof, respectively, include the amino acid sequences of SEQ ID NO:234-235, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A017. In one embodiment, the VH CDR1, VH CDR2, VH CDR3, VL CDR1, VL CDR2, and VL CDR3, respectively, include the amino acid sequences of SEQ ID NO:362-367. In one embodiment, the VH and VL of the antibody or antigen binding fragment thereof, respectively, include the amino acid sequences of SEQ ID NO:236-237, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A018. In one embodiment, the VH CDR1, VH CDR2, VH CDR3, VL CDR1, VL CDR2, and VL CDR3, respectively, include the amino acid sequences of SEQ ID NO:368-373. In one embodiment, the VH and VL of the antibody or antigen binding fragment thereof, respectively, include the amino acid sequences of SEQ ID NO:238-239, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A019. In one embodiment, the VH CDR1, VH CDR2, VH CDR3, VL CDR1, VL CDR2, and VL CDR3, respectively, include the amino acid sequences of SEQ ID NO:374-379. In one embodiment, the VH and VL of the antibody or antigen binding fragment thereof, respectively, include the amino acid sequences of SEQ ID NO:240-241, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A020. In one embodiment, the VH CDR1, VH CDR2, VH CDR3, VL CDR1, VL CDR2, and VL CDR3, respectively, include the amino acid sequences of SEQ ID NO:380-385. In one embodiment, the VH and VL of the antibody or antigen binding fragment thereof, respectively, include the amino acid sequences of SEQ ID NO:242-243, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A021. In one embodiment, the VH CDR1, VH CDR2, VH CDR3, VL CDR1, VL CDR2, and VL CDR3, respectively, include the amino acid sequences of SEQ ID NO:386-391. In one embodiment, the VH and VL of the antibody or antigen binding fragment thereof, respectively, include the amino acid sequences of SEQ ID NO:244-245, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A022. In one embodiment, the VH CDR1, VH CDR2, VH CDR3, VL CDR1, VL CDR2, and VL CDR3, respectively, include the amino acid sequences of SEQ ID NO:392-397. In one embodiment, the VH and VL of the antibody or antigen binding fragment thereof, respectively, include the amino acid sequences of SEQ ID NO:246-247, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A023. In one embodiment, the VH CDR1, VH CDR2, VH CDR3, VL CDR1, VL CDR2, and VL CDR3, respectively, include the amino acid sequences of SEQ ID NO:398-403. In one embodiment, the VH and VL of the antibody or antigen binding fragment thereof, respectively, include the amino acid sequences of SEQ ID N0248-249, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A024. In one embodiment, the VH CDR1, VH CDR2, VH CDR3, VL CDR1, VL CDR2, and VL CDR3, respectively, include the amino acid sequences of SEQ ID NO:404-409. In one embodiment, the VH and VL of the antibody or antigen binding fragment thereof, respectively, include the amino acid sequences of SEQ ID NO:250-251, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A025. In one embodiment, the VH CDR1, VH CDR2, VH CDR3, VL CDR1, VL CDR2, and VL CDR3, respectively, include the amino acid sequences of SEQ ID NO:410-415. In one embodiment, the VH and VL of the antibody or antigen binding fragment thereof, respectively, include the amino acid sequences of SEQ ID NO:252-253, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A026. In one embodiment, the VH CDR1, VH CDR2, VH CDR3, VL CDR1, VL CDR2, and VL CDR3, respectively, include the amino acid sequences of SEQ ID NO:416-421. In one embodiment, the VH and VL of the antibody or antigen binding fragment thereof, respectively, include the amino acid sequences of SEQ ID NO:254-255, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A027. In one embodiment, the VH CDR1, VH CDR2, VH CDR3, VL CDR1, VL CDR2, and VL CDR3, respectively, include the amino acid sequences of SEQ ID NO:422-427. In one embodiment, the VH and VL of the antibody or antigen binding fragment thereof, respectively, include the amino acid sequences of SEQ ID NO:256-257, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A028. In one embodiment, the VH CDR1, VH CDR2, VH CDR3, VL CDR1, VL CDR2, and VL CDR3, respectively, include the amino acid sequences of SEQ ID NO:428-433. In one embodiment, the VH and VL of the antibody or antigen binding fragment thereof, respectively, include the amino acid sequences of SEQ ID NO:258-259, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A029. In one embodiment, the VH CDR1, VH CDR2, VH CDR3, VL CDR1, VL CDR2, and VL CDR3, respectively, include the amino acid sequences of SEQ ID NO:434-439. In one embodiment, the VH and VL of the antibody or antigen binding fragment thereof, respectively, include the amino acid sequences of SEQ ID NO:260-261, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A030. In one embodiment, the VH CDR1, VH CDR2, VH CDR3, VL CDR1, VL CDR2, and VL CDR3, respectively, include the amino acid sequences of SEQ ID NO:440-445. In one embodiment, the VH and VL of the antibody or antigen binding fragment thereof, respectively, include the amino acid sequences of SEQ ID NO:262-263, or those having at least 70%, 75%, 80%, 85%, 90% or 95% sequence identity thereto while retaining the corresponding CDR sequences.

In one embodiment, the VH CDR1, VH CDR2, VH CDR3, VL CDR1, VL CDR2, and VL CDR3 are those of antibody A031. In one embodiment, the VH CDR1, VH CDR2, VH CDR3, VL CDR1, VL CDR2, and VL CDR3, respectively, include the amino acid sequences of SEQ ID NO:446-451. In one embodiment, the VH and VL of the antibody or antigen binding fragment thereof, respectively, include the amino acid sequences of SEQ ID NO:264-265, or those having at least 70%, 75%, 80%, 85%, 90% or 95% sequence identity thereto while retaining the corresponding CDR sequences.

In certain aspects, the antibody or antigen-binding fragment thereof can include 1, 2, and/or 3 CDRs from the variable heavy chain or variable light chain as disclosed in Table 4. The CDR may be one that has been determined by Kabat, IMGT, or Chothia. In further embodiments, a polypeptide may have CDRs that have 1, 2, and/or 3 amino acid changes (addition of 1 or 2 amino acids, deletions or 1 or 2 amino acids or substitution) with respect to these 1, 2, or 3 CDRs.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the reference protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

### Nucleic Acids

Also provided herein are nucleic acid or polynucleotide molecules that encode the antibody or antigen-binding fragment thereof described herein. The nucleic acids may be present, for example, in whole cells, in a cell lysate, or in a partially purified or substantially pure form.

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### III. Treatments

As demonstrated in the experimental examples, antibodies or their antigen-binding fragments that bind to Domain A of a PLXDC protein can block Domain A's inhibitory function, leading to activation of PLXDC signaling. Such PLXDC signaling can cause necrosis of the targeted cell. As PLXDC is commonly expressed in pathogenic blood vessels the killing of which can help treating associated diseases, these antibodies and antigen-binding fragments thereof can be effective in treating such diseases.

Accordingly, in one embodiment, the present disclosure provides a method for inhibiting the growth of or killing cells in a pathogenic blood vessel with an antibody or antigen-binding fragment thereof disclosed herein. In some embodiments, the antibody or antigen-binding fragment thereof disclosed herein binds Domain A of a PLXDC protein.

Pathogenic blood vessels are blood vessels that are not involved in the vascularization of normal organs but in the pathogenic tissues, such as the new blood vessels that drive vision diseases or the blood vessels in tumors that tumor depend on to survive. “Pathogenic blood vessel,” in some embodiments, refers to an existing blood vessel that may have vascularized a diseased tissue, for instance, a tumor. In other embodiments, a pathogenic blood vessel may be a blood vessel that is a newly formed blood vessel involved in disease onset and/or progression of, for example, cancer, diabetic retinopathy, age-related macular degeneration (AMD), retinopathy of prematurity and/or any other diseases having etiologies associated with angiogenesis.

In some embodiments, the pathogenic blood vessel is in a patient in need of a treatment. In some embodiments, the pathogenic blood vessel is associated with a disease or disorder, such as diabetic retinopathy, age-related macular degeneration (AMD), retinopathy of prematurity, cancer and combinations thereof.

In some aspects, provided herein are methods of treating cancer, as well as other diseases and disorder characterized with pathogenic blood vessels expressing PLXDC. In some embodiments, the method entails administering an antibody or fragment disclosed herein directly to the subject (e.g., by administering the antibody or fragment to the subject locally or systemically).

In some aspects, provided herein are methods of treating cancer or inducing necrosis (e.g., coagulative necrosis) in a subject. In some embodiments, the method entails administering an antibody or fragment disclosed herein directly to the subject (e.g., by administering the antibody or fragment to the subject locally or systemically).

The present disclosure provides methods and compositions for treating pathogenic blood vessel disorders such as diabetic retinopathy, age-related macular degeneration (AMD), retinopathy of prematurity, or cancer. In some embodiments, the method entails administering an antibody or fragment disclosed herein directly to the subject (e.g., by administering the antibody or fragment to the subject locally or systemically).

As noted, the present technology not only can inhibit growth of new tumor blood vessels, but can also kill existing tumor blood vessels, thereby treating the tumor. In some embodiments, therefore, a tumor patient that can benefit from the present treatment is one that has a tumor that has undergone tumor angiogenesis. In some embodiments, the tumor comprises a vascularized tumor. In some embodiments, the tumor being treat has a diameter that is greater than about 0.1, 0.2, 0.3, 0.4, 0.5, 1, 1.5, 2, 2.5, 3, 4, 5, 6, 7, 8, 9 or 10 cm (or any derivable range therein). In some embodiments, the tumor already contains tumor blood vessels.

In some embodiments, the tumor does not have a known tumor surface marker as target for immunotherapy. In some embodiments, the tumor does not contain a mutant gene that serves as a target for tumor therapy. In some embodiments, the therapy of the present disclosure does not include inducing antibody-dependent cell-mediated cytotoxicity (ADCC). In some embodiments, a therapeutic agent of the present disclosure does not induce ADCC.

In some embodiments, the patient suffers from a cancer such as, polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, the methods described herein may be used to treat any cancerous or pre-cancerous tumor, such as a solid tumor. Cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma or immunoproliferative small intestinal disease.

In some embodiments, the subject has cancer, preferably comprising a solid tumor. An agent disclosed herein may be administered locally to the tumor. In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngreal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor. In some embodiments, a compound and/or composition described herein may be administered parenterally, at or near the site of a tumor, or distant from the site of the tumor.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

The administration of one or more antibodies or fragments as described herein may result in at least a 10% decrease (e.g., at least 15%, 20%, 25%, 30%, 35%, 40%, 45%, 50%, 55%, 60%, 65%, 70%, 75%, 80%, 85%, 90% or even 100% decrease in one or more symptoms of a disease or condition, such as a decrease in tumor size.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Ex Vivo Tumor Angiogenesis Model

This example tested an ex vivo tumor angiogenesis model, which demonstrates that agents that activate PLXDC can effectively kill existing blood vessels. The following protocol describes the Ex Vivo Tumor Angiogenesis Model.

### Protocol:

1. The day before the experiment, all necessary tools were sprayed with 70% ethanol and sterilized under UV light overnight, including blade, dissecting and micro-dissecting scissors and biceps. 24-well dishes were placed at 4° C. to pre-chill plates and Matrigel was thawed, 24 hours before tumor dissection.

2. 70% ethanol was sprayed on working bench. Two sterile petri dishes were prepared with 10 ml sterile PBS. The following steps 3 and 4 are necessary for mouse tumor models. For fresh human tumor, steps 3 and 4 are skipped.

3. The tumor-bearing mice were euthanized. 70% ethanol was sprayed on the mouse and the tumor was removed using sterilized dissecting tools (avoid the fur). The tumor was rinsed in petri dish with sterile PBS to remove ethanol and fur. The tumor was transferred to a new petri dish with PBS for dissection. The dish is placed on ice.

4. Pre-chilled sterile pipet tips were used to seed regular Matrigel in 24 well plates on ice. Matrigel (30 μl) was dropped in the middle of each well without touching the edge of the well (avoid introducing bubbles if possible).

5. The tumor was cut in halves using the sterile blade. Healthy tumor tissue that is not necrotic and is within the tumor capsule was identified and isolated. The healthy tumor tissue was cut into small pieces. For instance, a suitable size for the tumor tissue is 0.5 mm (H)×0.5 mm (L)×0.3 mm (D) with a total volume of 0.075 mm³.

6. Each tumor piece was gently transferred and embedded in the Matrigel drop in the 24-well plate. The embedded piece was placed in the bottom and middle of each Matrigel drop. The plate was kept on ice all the time.

7. After seeding the tumor pieces, plates were incubated in a 37° C. cell culture incubator without medium for 10 minutes in order for the Matrigel to solidify.

8. Endothelial Growth Medium (0.5 ml) was added to each well and incubated at 37° C. with 5% CO₂. Treatment was not added until the new endothelial cells was grown out of the tumor or were larger than 3 mm in diameter. This usually takes 4 days for the LL2 Lewis lung cancer model and 7 days for the CT26 colon cancer model. For human tumor models, the growth time is typically 2-3 weeks, depending on the tumor type. Media was changed every 4 days during prolonged culture. Typically, when the tumor tissue grows to 2 mm in diameter, it was good for drug testing. A size of about 3 mm in diameter can make visualization easier.

9. When the assay was ready to be analyzed for cell death (e.g., 48 hours after drug addition), the dye mixture was prepared by mixing 6 μl of green dye to stain live cells (5 mg/ml Fluorescein diacetate or FDA in DMSO) with 30 μl of red dye (2.5 mg/ml Propidium iodide or PI in PBS) to stain dead cells in an Eppendorf tube. The FDA dye needs to be stored frozen in a −20° C. freezer because it has a labile ester bond.

10. 1 μl of the dye mixture was added to each well of the 24-well dish. It is usually preferred to do one 24-well dish at a time given the amount of time needed to take pictures (the green dye is not as stable in the cells in the long term).

11. The dish was gently rocked a few times to mix the dye with the media in the wells and incubate the dish at 37° C. for 10 min (too long incubation can make the green signal too intense).

12. Each well was washed with 0.5 ml of sterile PBS and then 0.5 ml of phenol red free SFM was added to each well. Alternatively 0.5 ml of regular Endothelial Cell Growth Media can be added to each well if this well needs to be continuously maintained after the experiment.

13. An inverted microscope using the 2× objective lens was used to observe morphological changes in the experimental wells.

14. Taking pictures in the red and green channels would allow not only the recording of the results but also more accurate quantitation of the results. To take pictures for all the wells, first a well that has robust red and green signals was picked. A picture at the red channel using the optimal setting (remember this setting) was taken and then a picture at the green channel using another optimal setting (remember this setting) was taken. The final picture is the merged picture of the red and green channels. Pictures of all other wells were taken in each channel using the same settings so that different wells can be compared.

Using the ex vivo model described above, the inventors designed and screened antibodies of PLXDC1 for their ability to kill pathogenic blood vessels. The results are provided below.

### Example 2. Generation ofPLXDC-Activating Antibodies

This example describes the generation of monoclonal antibodies that bind and activate the PLXDC proteins.

At a first step, a customized human antibody library was created, which contained about 10 billion antibody clones. To screen for activating antibodies from this library, a screening assay was established with the human PLXDC 1 protein (biotinylated at a biotin/PLXDC 1 ratio of about 2.7) in the presence of an activating small molecule compound (e.g., compound 369 described in WO 2021/076930).

About 100 human antibody clones were identified with this method that preferentially bound to the small molecule-activated PLXDC 1. The VH/VL sequences of selected ones are presented in Table 3 below.

TABLE 3
Select PLXDC1-Activating Antibodies

| | | SEQ ID |
|---|---|---|
| Name | Sequence | NO: |
| 1-A1_VH | EVQLLESGGGLVQPGGSLRLSCAASGFTFTSYAMSWVRQAPGKGLEWVSAISAG | 4 |
| | GGYTYYADSVKGRFTISRDNSKNTLYLQMNSLRAEDTAVYYCARYDYNSYFDSW | |
| | GQGTLVTVSS | |
| 1-A1_VL | QSTQPPSVSGAPGQRVTISCTGSSSNIGAGYDVHWYQQLPGTAPKLLIYGNNNR | 5 |
| | PSGVPDRFSGSKSGTSASLAITGLQAEDEADYYCQSYDRSLSGWVFGGGTKLTV | |
| | L | |
| 1-A5_VH | QVQLVESGGGVVQPGRSLRLSCAASGYAFSSYGMHWVRQAPGKGLEWVAVISHS | 6 |
| | GSNKYYANSVKGRFTISRDNSKNTLYLQMNSLRAEDTAVYYCARGLAGWEVYPF | |
| | DVWGQGTLVTVSS | |
| 1-A5_VL | QSTQPPSVSGAPGQRVTISCTGSSSNIGAGFDVHWYQQLPGTAPKLLIYDNINR | 7 |
| | PSGVPDRFSGSKSGTSASLAITGLQAEDEADYYCQSYDYSLRVWVFGGGTKLTV | |
| | L | |
| 1- | EVQLLESGGGLVQPGGSLRLSCAASGFTFSNYAMSWVRQAPGKGLEWVSTISGG | 8 |
| H10_VH | GTYYADSVKGRFTISRDNSKNTLYLQMNSLRAEDTAVYYCARSRYWYTKLISYT | |
| | YGMDVWGQGTLVTVSS | |
| 1- | QSVLTQPPSVSGAPGQRVTISCTGSSSNIGATYDVHWYQQLPGTAPKLLIYVNN | 9 |
| H10_VL | NRPSGVPDRFSGSKSGTSASLAITGLQAEDEADYYCQSYDSSLRAWVFGGGTKL | |
| | TVI | |
| 2-B4_VH | QVQLVESGGGVVQPGRSLRLSCAASGFTFSNYGMHWVRQAPGKGLEWVAVISYH | 10 |
| | GRNEYYADSVKGRFTISRDNSKNTLYLQMNSLRAEDTAVYYCARGDDQDAFDPW | |
| | GQGTLVTVSS | |
| 2-B4_VL | DIQMTQSPSSLSASVGDRVTITCRASQSISNYLNWYQQKPGKAPKLLIYAASSL | 11 |
| | QSGVPSRFSGSGSGTDFTLTISSLQPEDFATYYCQQSFSAPYTFGQGTKVEIK | |
| 2-B5_VH | QVQLVESGGGVVQPGRSLRLSCAASGFIFSDYDMHWVRQAPGKGLEWVAVISHS | 12 |
| | GSNKYYADSVKGRFTISRDNSKNTLYLQMNSLRAEDTAVYYCARDRSVYLYYGY | |
| | HYYEGFDVWGQGTLVTVSS | |
| 2-B5_VL | EIVMTQSPATLSVSPGERATLSCRASQSVYNNLAWYQQKPGQAPRLLIYGASTR | 13 |
| | ATGIPARFSGSGSGTEFTLTISSLQSEDFAVYYCQQYNDLPLTFGQGTKVEIK | |
| 2-F7_VH | EVQLLESGGGLVQPGGSLRLSCAASGFTFSDYAMSWVRQAPGKGLEWVSSIYTS | 14 |
| | GGRTYYADSVKGRFTISRDNSKNTLYLQMNSLRAEDTAVYYCARGYRYFDHWGQ | |
| | GTLVTVSS | |
| 2-F7_VL | EIVMTQSPATLSVSPGERATLSCRASQSVRNNLAWYQQKPGQAPRLLIYGASTR | 15 |
| | ATGIPARFSGSGSGTEFTLTISSLQSEDFAVYYCQQYYTWPRTFGQGTKVEIK | |
| 2-F8_VH | EVQLLESGGGLVQPGGSLRLSCAASGFTFPNYAMSWVRQAPGKGLEWVSTIYGR | 16 |
| | GERTYYADSVKGRFTISRDNSKNTLYLQMNSLRAEDTAVYYCARVSVVWSYAFD | |
| | YWGQGTLVTVSS | |
| 2-F8_VL | DIQMTQSPSSLSASVGDRVTITCRASQDISTYLNWYQQKPGKAPKLLIYAASSL | 17 |
| | QSGVPSRFSGSGSGTDFTLTISSLQPEDFATYYCQQSYSYPLTFGQGTKVEIK | |
| 2-G4_VH | QLQLQESGPGLVKPSETLSLTCTVSGGSISSSNYYWGWIRQPPGKGLEWIGSVY | 18 |
| | YTGRTYYNPSLKSRVTISVDTSKNQFSLRLSSVTAADTAVYYCARVFPYGAVDV | |
| | WGQGTLVTVSS | |
| 2-G4_VL | EIVLTQSPGTLSLSPGERATLSCRASQSVSGSYLAWYQQKPGQAPRLLIYGASS | 19 |
| | RATGIPDRFSGSGSGTDFTLTISRLEPEDFAVYYCQQYGGSLPYTFGQGTKVEI | |
| | K | |
| 2-H2_VH | QVQLVESGGGVVQPGRSLRLSCAASGFTFSDYGMHWVRQAPGKGLEWVAVISYE | 20 |
| | GSNEYYADSVKGRFTISRDNSKNTLYLQMNSLRAEDTAVYYCARGSEGSGFDVW | |
| | GQGTLVTVSS | |
| 2-H2_VL | DIQMTQSPSSLSASVGDRVTITCRASQRISNYLNWYQQKPGKAPKLLIYAASSL | 21 |
| | QSGVPSRFSGSGSGTDFTLTISSLQPEDFATYYCQQSYSPPYTFGQGTKVEIK | |
| 2-H9_VH | EVQLLESGGGLVQPGGSLRLSCAASGFTFSTYAMSWVRQAPGKGLEWVSSIYGS | 22 |
| | GGKTYYADSVKGRFTISRDNSKNTLYLQMNSLRAEDTAVYYCARTDYSLYYQFD | |
| | YWGQGTLVTVSS | |
| 2-H9_VL | DIQMTQSPSSLSASVGDRVTITCRASQSISRYLNWYQQKPGKAPKLLIYAASSL | 23 |
| | QSGVPSRFSGSGSGTDFTLTISSLQPEDLATYYCQQSYSYPVTFGQGTKVEIK | |
| 3-A7_VH | QVQLVESGGGVVQPGRSLRLSCAASGFTFTNYGMHWVRQAPGKGLEWVAVISED | 24 |
| | GSNKYYANSVKGRFTISRDNSKNTLYLQMNSLRAEDTAVYYCARGGDEDGEDVW | |
| | GQGTLVTVSS | |
| 3-A7_VL | DIQMTQSPSSLSASVGDRVTITCRASQSISSYLNWYQQKPGKAPKLLIYAASSL | 25 |
| | QSGVPSRFSGSGSGTDFTLTISSLQPEDFATYYCQQSYRPPYTFGQGTKVEIK | |
| 3-A9_VH | EVQLLESGGGLVQPGGSLRLSCAASGFTFSNYAMSWVRQAPGKGLEWVSSITGS | 26 |
| | GEYTNYADSVKGRFTISRDNSKNTLYLQMNSLRAEDTAVYYCAREVADYWDGGV | |
| | YYYYDGGFDVWGQGTLVTVSS | |
| 3-A9_VL | EIVLTQSPGTLSLSPGERATLSCRASQSVSSNYLAWYQQKPGQAPRLLIYGASS | 27 |
| | RATGIPDRFSGSGSGTDFTLTISRLEPEDFAVYYCQQYGRSAPITFGQGTKVEI | |
| | K | |
| 3-B3_VH | EVQLLESGGGLVQPGGSLRLSCAASGFTFSSYAMSWVRQAPGKGLEWVSTISGT | 28 |
| | GGRTYYADSVKGRFTISRDNSKNTLYLQMNSLRAEDTAVYYCARVDHYSGYAFD | |
| | LWGQGTLVTVSS | |
| 3-B3_VL | DIQMTQSPSSLSASVGDRVTITCRASQSISSYLNWYQQKPGKAPKLLIYAASSL | 29 |
| | QSGVPSRFSGSGSGTDFTLTISSLQPEDFATYYCQQSYSYPLTFGQGTKVEIK | |
| 3-C4_VH | EVQLLESGGGLVQPGGSLRLSCAASGFTFSSYAMSWVRQAPGKGLEWVSTIYGG | 30 |
| | GSYTYYADSVKGRFTISRDNSKNTLYLQMNSLRAEDTAVYYCARAEVDDYWYLY | |
| | MDVWGQGTLVTVSS | |
| 3-C4_VL | DIQMTQSPSSLSASVGDRVTITCRASQDIGNYLNWYQQKPGKAPKLLIYAASSL | 31 |
| | QSGVPSRFSGSGSGTDFTLTISSLQPEDFATYYCQQSYTFPLTFGQGTKVEIK | |
| 3-C6_VH | EVQLLESGGGLVQPGGSLRLSCAASGFTFSTYAMSWVRQAPGKGLEWVSTIYGS | 32 |
| | GGRTYYADSVKGRFTISRDNSKNTLYLQMNSLRAEDTAVYYCARTDHYSYYVMD | |
| | YWGQGTLVTVSS | |
| 3-C6_VL | DIQMTQSPSSLSASVGDRVTITCRASQSISKYLNWYQQKPGKAPKLLIYSASSL | 33 |
| | QSGVPSRFSGSGSGTDFTLTISSLQPEDCATYYCQQSYSFPLTFGQGTKVEIK | |
| 3-C8_VH | EVQLLESGGGLVQPGGSLRLSCAASGFTFSDYAMSWVRQAPGKGLEWVSAIYGS | 34 |
| | GSYTYYADSVKGRFTISRDNSKNTLYLQMNSLRAEDTAVYYCARTAYSYPYVYF | |
| | DFWGQGTLVTVSS | |
| 3-C8_VL | DIQMTQSPSSLSASVGDRVTITCRASQTITSYLNWYQQKPGKAPKLLIYAASSL | 35 |
| | QSGVPSRFSGSGSGTDFTLTISSLQPEDFATYYCQQSYTYPLTFGQGTKVEIK | |
| 3-C9_VH | EVQLLESGGGLVQPGGSLRLSCAASGFTFGNYAMSWVRQAPGKGLEWVSVISGG | 36 |
| | GTNTYYADSVKGRFTISRDNSKNTLYLQMNSLRAEDTAVYYCARSDSYYGVYDG | |
| | YYYGMDVWGQGTLVTVSS | |
| 3-C9_VL | DIQMTQSPSSLSASVGDRVTITCRASQTISRYLNWHQQKPGKAPKLLIYAASSL | 37 |
| | QSGVPSRFSGSGSGTDFTLTISSLQPEDFATYYCQQTYSTPWTFGQGTKVEIK | |
| 3- | EVQLLESGGGLVQPGGSLRLSCAASGFTFSSYAMSWVRQAPGKGLEWVSTIYAS | 38 |
| C11_VH | GATTYYADSVKGRFTISRDNSKNTLYLQMNSLRAEDTAVYYCARLKLYHAAFDI | |
| | WGQGTLVTVSS | |
| 3- | DIQMTQSPSSLSASVGDRVTITCRASQDISSYLNWYQQKPGKAPKLLIYTASSL | 39 |
| C11_VL | QSGVPSRFSGSGSGTDFTLTISSLQPEDFATYYCQQSYSYPLTFGQGTKVEIK | |
| 3- | EVQLLESGGGLVQPGGSLRLSCAASGFTFRDYAMSWVRQAPGKGLEWVSTIYGS | 40 |
| C12_VH | GSRTYYADSVKGRFTISRDNSKNTLYLQMNSLRAEDTAVYYCARVDHVHGYAFD | |
| | YWGQGTLVTVSS | |
| 3- | DIQMTQSPSSLSASVGDRVTITCRASQSISKYLNWYQQKPGKAPKLLIYAASSL | 41 |
| C12_VL | QSGVPSRFSGSGSGTDFTLTISSLQPEDFATYYCQQSYSFPPTFGQGTKVEIK | |
| 3-D3_VH | EVQLLESGGGLVQPGGSLRLSCAASGFTFSNYAMSWVRQAPGKGLEWVSTIYES | 42 |
| | GGYTYYADSVKGRFTISRDNSKNTLYLQMNSLRAEDTAVYYCARVESYGYGEYI | |
| | TYNYYGFDVWGQGTLVTVSS | |
| 3-D3_VL | EIVLTQSPGTLSLSPGERATLSCRASQSVATGYLAWYQQKPGQAPRLLIYGASS | 43 |
| | RATGIPDRFSGSGSGTDFTLTISRLEPEDFAVYYCQQYESSPPFTFGQGTKVEI | |
| | K | |
| 3-D6_VH | QVQLVQSGAEVKKPGSSVKVSCKASGGTFTSYGIHWVRQAPGQGLEWMGRIVPI | 44 |
| | LGTTNYAQKFQGRVTITADESTSTAYMELSSLRSEDTAVYYCARDPAVTLDTYV | |
| | YADHGFDVWGQGTLVTVSS | |
| 3-D6_VL | EIVMTQSPATLSVSPGERATLSCRASQSVYNNLAWYQQKPGQAPRLLIYGASTR | 45 |
| | ATGIPARFSGSGSGTEFTLTISSLQSEDFAVYYCQQYNDWPLTFGQGTKVEIK | |
| 3-D7_VH | EVQLLESGGGLVQPGGSLRLSCAASGFTFKNYAMSWVRQAPGKGLEWVSGISEG | 46 |
| | GANTYYADSVKGRFTISRDNSKNTLYLQMNSLRAEDTAVYYCAREPPKYGAYSY | |
| | YGYGFDPWGQGTLVTVSS | |
| 3-D7_VL | EIVLTQSPGTLSLSPGERATLSCRASQSVGSNYLAWYQQKPGQAPRLLIYGASS | 47 |
| | RATGIPDRFSGSGSGTDFTLTISRLEPEDFAVYYCQQYGSSPYTFGQGTKVEIK | |
| 3- | EVQLLESGGGLVQPGGSLRLSCAASGFTFKGYAMSWVRQAPGKGLEWVSSISVS | 48 |
| D12_VH | GAGTYYADSVKGRFTISRDNSKNTLYLQMNSLRAEDTAVYYCARYVRGVSFDVW | |
| | GQGTLVTVSS | |
| 3- | EIVLTQSPGTLSLSPGERATLSCRASQSVSSSYLAWYQQKPGQAPRLLIYGASS | 49 |
| D12_VL | RATGIPDRFSGSGSGTDFTLTISRLEPEDFAVYYCQQYETSPPVTFGQGTKVEI | |
| | K | |
| 3-E2_VH | EVQLLESGGGLVQPGGSLRLSCAASGFTFSNYAMSWVRQAPGKGLEWVSAIGGS | 50 |
| | GGGTYYADSVKGRFTISRDNSKNTLYLQMNSLRAEDTAVYYCARYGWEYQAFDY | |
| | WGQGTLVTVSS | |
| 3-E2_VL | DIQMTQSPSSLSASVGDRVTITCRASQSISRYLNWYQQKPGKAPKLLIYAASSL | 51 |
| | QSGVPSRFSGSGSGTDFTLTISSLQPEDFATYYCQQSYSTPWTFGQGTKVEIK | |
| 3-E5_VH | EVQLLESGGGLVQPGGSLRLSCAASGFTFSNYAMSWVRQAPGKGLEWVSGITAG | 52 |
| | GGNTYYADSVKGRFTISRDNSKNTLYLQMNSLRAEDTAVYYCARIAGLFDGFDV | |
| | WGQGTLVTVSS | |
| 3-E5_VL | EIVLTQSPGTLSLSPGERATLSCRASQSVRSSYLAWYQQKPGQAPRLLIYGASS | 53 |
| | RATGIPDRFSGSGSGTDFTLTISRLEPEDFAVYYCQQYASLPLTFGQGTKVEIK | |
| 3-E7_VH | EVQLLESGGGLVQPGGSLRLSCAASGFTFSNYAMSWVRQAPGKGLEWVSGISAS | 54 |
| | GGGTYYADSVKGRFTISRDNSKNTLYLQMNSLRAEDTAVYYCARSSRYGDTFDV | |
| | WGQGTLVTVSS | |
| 3-E7_VL | EIVMTQSPATLSVSPGERATLSCRASLSVGSNLAWYQQKPGQAPRLLIYGASTR | 55 |
| | ATGIPARFSGSGSGTEFTLTISSLQSEDFAVYYCQQYNNLPLTFGQGTKVEIK | |
| 3-E8_VH | EVQLLESGGGLVQPGGSLRLSCAASGFTFTDYAMSWVRQAPGKGLEWVSGISVS | 56 |
| | GGYTYYADSVKGRFTISRDNSKNTLYLQMNSLRAEDTAVYYCARGLYAHHTYYY | |
| | GGFDYWGQGTLVTVSS | |
| 3-E8_VL | EIVMTQSPATLSVSPGERATLSCRASQSVGSNLAWYQQKPGQAPRLLIYGASTR | 57 |
| | ATGIPARFSGSGSGTEFTLTISSLQSEDFAVYYCQQYHNWPPTFGQGTKVEIK | |
| 3-E9_VH | EVQLLESGGGLVQPGGSLRLSCAASGFTFRSYAMSWVRQAPGKGLEWVSTIYGT | 58 |
| | GEDTYYADSVKGRFTISRDNSKNTLYLQMNSLRAEDTAVYYCARDYFWSPRGSI | |
| | YDYDYFDVWGQGTLVTVSS | |
| 3-E9_VL | DIQMTQSPSSLSASVGDRVTITCRASQSISRYLNWYQQKPGKAPKLLIYSASSL | 59 |
| | QSGVPSRFSGSGSGTDFTLTISSLQPEDFATYYCQQYFSAPYTFGQGTKVEIK | |
| 3-F5_VH | EVQLLESGGGLVQPGGSLRLSCAASGFTFKGYAMSWVRQAPGKGLEWVSSISVS | 60 |
| | GAGTYYADSVKGRFTISRDNSKNTLYLQMNSLRAEDTAVYYCARYVRGVSFDVW | |
| | GQGTLVTVSS | |
| 3-F5_VL | EIVLTQSPGTLSLSPGERATLSCRASQSVSSNYLAWYQQKPGQAPRLLIYGASS | 61 |
| | RATGIPDRFSGSGSGTDFTLTISRLEPEDFAVYYCQQYGSSPAVTFGQGTKVEI | |
| | K | |
| 3-F6_VH | EVQLLESGGGLVQPGGSLRLSCAASGFTFSDYAMSWVRQAPGKGLEWVSAISSS | 62 |
| | GGTTYYADSVKGRFTISRDNSKNTLYLQMNSLRAEDTAVYYCARDAGWSYYGLW | |
| | IYYYYMDVWGQGTLVTVSS | |
| 3-F6_VL | EIVLTQSPGTLSLSPGERATLSCRASQSVRSNYLAWYQQKPGQAPRLLIYGASS | 63 |
| | RATGIPDRFSGSGSGTDFTLTISRLEPEDFAVYYCQQYGASPQTFGQGTKVEIK | |
| 3- | EVQLLESGGGLVQPGGSLRLSCAASGFTFKDYAMSWVRQAPGKGLEWVSGISGS | 64 |
| F12_VH | GGYTYYADSVKGRFTISRDNSKNTLYLQMNSLRAEDTAVYYCARKLGGFDENYT | |
| | YGMDVWGQGTLVTVSS | |
| 3- | EIVLTQSPGTLSLSPGERATLSCRASQSVSSSYLAWYQQKPGQAPRLLIYGASS | 65 |
| F12_VL | RATGIPDRFSGSGSGTDFTLTISRLEPEDFAVYYCQQYGSSPEYTFGQGTKVEI | |
| | K | |
| 3-G4_VH | EVQLLESGGGLVQPGGSLRLSCAASGFTFSTYAMSWVRQAPGKGLEWVSGISAG | 66 |
| | GGARTYYADSVKGRFTISRDNSKNTLYLQMNSLRAEDTAVYYCARVHHEGYYSL | |
| | DVWGQGTLVTVSS | |
| 3-G4_VL | DIQMTQSPSSLSASVGDRVTITCRASQTISNYLNWYQQKPGKAPKLLIYAASSL | 67 |
| | QSGVPSRFSGSGSGTDFTLTISSLQPEDFATYYCQQSYTYPLTFGQGTKVEIK | |
| 3-G5_VH | EVQLLESGGGLVQPGGSLRLSCAASGFTFSSYAMSWVRQAPGKGLEWVSVIYGS | 68 |
| | GGRTYYADSVKGRFTISRDNSKNTLYLQMNSLRAEDTAVYYCARHDPRYQLDVW | |
| | GQGTLVTVSS | |
| 3-G5_VL | DIQMTQSPSSLSASVGDRVTITCRASQSIGSYLNWYQQKPGKAPKLLIYTASSL | 69 |
| | QSGVPSRFSGSGSGTDFTLTISSLQPEDFATYYCQQSYTYPLTFGQGTKVEIK | |
| 3-G6_VH | QVQLVESGGGVVQPGRSLRLSCAASGFTFSDYGMHWVRQAPGKGLEWVAVISYD | 70 |
| | GSRKYYANSVKGRFTISRDNSKNTLYLQMNSLRAEDTAVYYCARGSYSDGFDVW | |
| | GQGTLVTVSS | |
| 3-G6_VL | DIQMTQSPSSLSASVGDRVTITCRASQSISRYLNWYQQKTGKAPKLLIYGASSL | 71 |
| | QSGVPSRFSGSGSGTDFTLTISSLQPEDFATYYCQQSYSTPYTFGQGTKVEIK | |
| 3-G7_VH | QVQLVQSGAEVKKPGSSVKVSCKASGGTFSTEYISWVRQAPGQGLEWMGRIIPV | 72 |
| | LGITNYAQKFQGRVTITADESTSTAYMELSSLRSEDTAVYYCARHLGPPYLVTY | |
| | SHGFDVWGQGTLVTVSS | |
| 3-G7_VL | EIVMTQSPATLSVSPGERATLSCRASQSLGTNLAWYQQKPGQAPRLLIYGASTR | 73 |
| | ATGIPARFSGSGSGTEFTLTISSLQSEDFAVYYCQQYYEAPPTFGQGTKVEIK | |
| 3-G8_VH | EVQLLESGGGLVQPGGSLRLSCAASGFTFSNYAMSWVRQAPGKGLEWVSAIYGS | 74 |
| | GGKTYYADSVKGRFTISRDNSKNTLYLQMNSLRAEDTAVYYCARLKYGFYQFDY | |
| | WGQGTLVTVSS | |
| 3-G8_VL | DIQMTQSPSSLSASVGDRVTITCRASQSIRNYLNWYEQKPGKAPKLLIYAASSL | 75 |
| | QSGVPSRFSGSGSGTDFTLTISSLQPEDFATYYCQQSYSYPLTFGQGTKVEIK | |
| 3-H1_VH | EVQLLESGGGLVQPGGSLKLSCAASGFTFSNYAMSWVRQAPGKGLEWVSVIYAG | 76 |
| | GARTYYADSVKGRFTISRDNSKNTLYLQMNSLRAEDTAVYYCARWGGDGFYAMD | |
| | YWGQGTLVTVSS | |
| 3-H1_VL | DIQMTQSPSSLSASVGDRVTITCRASQTISTYLNWYQQKPGKAPKLLIYAASIL | 77 |
| | QSGVPSRFSGSGSGTDFTLTISSLQPEDFATYYCQQSYTVPLTFGQGTKVEIK | |
| 3-H4_VH | EVQLLESGGGLVQPGGSLRLSCAASGFTFSNYAMSWVRQAPGKGLEWVSAISGS | 78 |
| | GGTTYYADSVKGRFTISRDNSKNTLYLQMNSLRAEDTAVYYCARIKKYGSAGYT | |
| | VGFDSWGQGTLVTVSS | |
| 3-H4_VL | EIVMTQSPATLSVSPGERATLSCRASQSVGSYLAWYQQKPGQAPRLLIYGASTR | 79 |
| | ATGIPARFSGSGSGTEFTLTISSLQSEDFAVYYCQQYYNAPITFGQGTKVEIK | |
| 4-A7_VH | QLQLQESGPGLVKPSETLSLTCTVSGGSISSSSYYWGWIRQPPGKGLEWIGNIY | 80 |
| | YTGTTYYNPSLKSRVTISVDTSKNQFSLKLSSVTAADTAVYYCARIKVPWYYSS | |
| | YYFDYWGQGTLVTVSS | |
| 4-A7_VL | QSVLTQPPSVSGAPGQRVTISCTGSSSNIGAGYDVHWYQQLPGTAPKLLIYGNK | 81 |
| | NRPSGVPDRFSGSKSGTSASLAITGLQAEDEADYYCQSYDTSLSAWVFGGGTKL | |
| | TVL | |
| 4-A8_VH | EVQLLESGGGLVQPGGSLRLSCAASGFTFKNYAMSWVRQAPGKGLEWVSAISAS | 82 |
| | GGYTYYADSVKGRFTISRDNSKNTLYLQMNSLRAEDTAVYYCARGRWGAFDYWG | |
| | QGTLVTVSS | |
| 4-A8_VL | QSVLTQPPSVSGAPGQRVTISCTGSSSNIGSGYDVHWYQQLPGTAPKLLIYGNR | 83 |
| | NRPSGVPDRFSGSKSGTSASLAITGLQAEDEADYYCQSYDSSLKTWVFGGGTKL | |
| | TVL | |
| 4-B1_VH | EVQLLESGGGLVQPGGSLKLSCAASGFTFSDYAMSWVRQAPGKGLEWVSGISRG | 84 |
| | GARTYYADSVKGRFTISRDNSKNTLYLQMNSLRAEDTAVYYCARYLFGHYMDWG | |
| | QGTLVTVSS | |
| 4-B1_VL | QSVLTQPPSVSGAPGQRVTISCTGSSSNIGAGYDVHWYQQLPGTAPKLLIYGNT | 85 |
| | NRPSGVPDRFSGSKSGTSASLAITGLQAEDEADYYCQSYDGRLGVSVFGGGTKL | |
| | TVL | |
| 4-B2_VH | QVQLVESGGGVVQPGRSLRLSCAASGFTFSRYAMHWVRQAPGKGLEWVAVISHS | 86 |
| | GSTKYYANSVKGRFTISRDNSKNTLYLQMNSLRAEDTAVYYCARSRGWRGFDSW | |
| | GQGTLVTVSS | |
| 4-B2_VL | QSVLTQPPSVSGAPGQRVTISCTGSSSNIGAYVVHWYQQLPGTAPKLLIYGNIN | 87 |
| | RPSGVPDRFSGSKSGTSASLAITGLQAEDEADYYCQSYDRTLSSYVFGGGTKLT | |
| | VL | |
| 4- | QVQLVESGGGVVQPGRSLRLSCAASGFAFSGYGMHWVRQAPGKGLEWVAVISHH | 88 |
| B11_VH | GSYKYYANSVKGRFTISRDNSKNTLYLQMNSLRAEDTAVYYCARGGADVGYFYS | |
| | TFYYYMDVWGQGTLVTVSS | |
| 4- | QSVLTQPPSVSGAPGQRVTISCTGSSSNIGAAFDVHWYQQLPGTAPKLLIYDNY | 89 |
| B11_VL | NRPSGVPDRFSGSKSGTSASLAITGLQAEDEADYYCQSYDSSLSVWVFGGGTKL | |
| | TVL | |
| 4- | EVQLLESGGGLVQPGGSLRLSCAASGFTFSNYAMSWVRQAPGKGLEWVSAISRG | 90 |
| D12_VH | GGYTYYADSVKGRFTISRDNSKNTLYLQMNSLRAEDTAVYYCARWGGDGFYAMD | |
| | YWGQGTLVTVSS | |
| 4- | QSVLTQPPSVSGAPGQRVTISCTGSSSNIGAGYDVHWYQQLPGTAPKLLIYANN | 91 |
| D12_VL | NRPSGVPDRFSGSKSGTSASLAITGLQAEDEADYYCQSYDYSLSGWVFGGGTKL | |
| | TVL | |
| 4-F4_VH | QLQLQESGPGLVKPSETLSLTCTVSGGSISSRGYYWAWIKQPPGKGLEWIGSIY | 92 |
| | YSGSTYYNPSLKSRVTISVDTSKNQFSLKLSSVTAADTAVYYCARLVSYGAYYV | |
| | FDYWGQGTLVTVSS | |
| 4-F4_VL | QSVLTQPPSVSGAPGQRVTISCTGSSSNIGAGYDVHWYQQLPGTAPKLLIYGNR | 93 |
| | NRPSGVPDRFSGSKSGTSASLAITGLQAEDEADYYCQSYDTRLSAWVFGGGTKL | |
| | TVL | |
| 4- | EVQLLESGGGLVRPGGSLRLSCAASGFTFSSYAMSWVRQAPGKGLEWVSAISGS | 94 |
| G12_VH | GGSTYYADSVKGRFTISRDNSKNTLYLQMNSLRAEDTAVYYCARDKRLTYWGQG | |
| | TMVTVSS | |
| 4- | QSVVTQPPSVSAAPGQKVTISCSGSSSNIGNNYVSWYQQLPGTAPKLLIYDNNK | 95 |
| G12_VL | RPSGIPDRFSGSKSGTSATLGITGLQTGDEADYYCGTWDSSLSARVFGGGTKVT | |
| | VL | |
| 5-C9_VH | QVQLVQSGAEVKKPGSSVKVSCKASGGTFSSYAINWVRQAPGQGLEWMGRIIPL | 96 |
| | LETADYAQKFQGRVTITADESTSTAYMELSSLRSEDTAVYYCAREWPGEYFDVW | |
| | GQGTLVTVSS | |
| 5-C9_VL | EIVLTQSPGTLSLSPGERATLSCRASQSVASSYLAWYQQKPGQAPRLLIYGASS | 97 |
| | RATGIPDRFSGSGSGTDFTLTISRLEPEDFAVYYCQQYGSSQLTFGQGTKVEIK | |
| 5-E2_VH | QVQLVESGGGVVQPGRSLRLSCAASGFTFRDYGMHWVRQAPGKGLEWVAVISYH | 98 |
| | GRNEYYANSVKGRFTISRDNSKNTLYLQMNSLRAEDTAVYYCARGAYGDDFDVW | |
| | GQGTLVTVSS | |
| 5-E2_VL | DIQMTQSPSSLSASVGDRVTITCRASQSISSYLNWYQQKTGKAPKLLIYAASSL | 99 |
| | QSGVPSRFSGSGSGTDFTLTISSLQPEDFATYYCQQSYSAPYTFGQGTKVEIK | |
| 5- | EVQLLESGGGLVQPGGSLRLSCAASGFTFSNYAMSWVRQAPGKGLEWVSTISGG | 100 |
| E12_VH | GRTTNYADSVKGRFTISRDNSKNTLYLQMNSLRAEDTAVYYCARSTPIPSYYPY | |
| | IYSYYFDVWGQGTLVTVSS | |
| 5 | EIVMTQSPATLSVSPGERATLSCRASQSVSNNLAWYQQKPGQAPRLLIYGASTR | 101 |
| E12_VL | ATGIPARFSGSGSGTEFTLTISSLQSEDFAVYYCQQYGNYPPTFGQGTKVEIK | |
| 6-G4_VH | EVQLLESGGGLVQPGGSLRLSCAASGFTFSDYAMSWVRQAPGKGLEWVSSISSS | 102 |
| | GANTYYADSGKGRFTISRDNSKNILYLQMNSLRAEDTAVYYCARSVVTWVTYAF | |
| | DYWGQGTLVTVSS | |
| 6-G4_VL | QSVLTQPPSVSGAPGQRVTISCTGSSSNIGAGYDVHWYQQLPGTAPKLLIYDNT | 103 |
| | NRPSGVPDRFSGSKSGTSASLAITGLQAEDEADYYCQSYDSGLSGWVFGGGTKL | |
| | TVL | |
| 6-H5_VH | EVQLLESGGGLVQPGGSLRLSCAASGFTFSNYAMSWVRQAPGKGLEWVSAISAT | 104 |
| | GGATYYADSVKGRFTISRDNSKNTLYLQMNSLRAEDTAVYYCARSVRERYTYYM | |
| | DYWGQGTLVTVSS | |
| 6-H5_VL | QSVLTQPPSVSGAPGQRVTISCTGSSSNIGAGYDVHWYQQLPGTAPKLLIYDNT | 105 |
| | NRPSGVPDRFSGSKSGTSASLAITGLQAEDEADYYCQSYDVSLGVWVFGGGTKL | |
| | TVL | |
| 8-A2_VH | QVQLVQSGAEVKKPGSSVKVSCKASGGTFSNYYISWVRQAPGQGLEWMGGIIPV | 106 |
| | FGVAHYAQKFQGRVTITADESTSTAYMELSSLRSEDTAVYYCARGYPGEYFDYW | |
| | GQGTLVTVSS | |
| 8-A2_VL | EIVLTQSPGTLSLSPGERATLSCRASQSVSSSYLAWYQQKPGQAPRLLIYAASS | 107 |
| | RATGIPDRFSGSGSGTDFTLTISRLEPEDFAVYYCQHYGGSPGYTFGQGTKVEI | |
| | K | |
| 8-A4_VH | EVQLLESGGGLVQPGGSLRLSCAASGFTFGSYAMSWVRQAPGKGLEWVSSIYAG | 108 |
| | GGRTYYADSVKGRFTISRDNSKNTLYLQMNSLRAEDTAVYYCARWGGDGFYAMD | |
| | YWGQGTLVTVSS | |
| 8-A4_VL | DIQMTQSPSSLSASVGDRVTITCRASQTISNYLNWYQQKPGKAPKLLIYGASSL | 109 |
| | QSGVPSRFSGSGSGTDFTLTISSLQPEDFATYYCQQSFTFPLTFGQGTKVEIK | |
| 8-A6_VH | EVQLLESGGGLVQPGGSLRLSCAASGFTFSDYAMSWVRQAPGKGLEWVSTIYSG | 110 |
| | GVRTYYADSVKGRFTISRDNSKNTLYLQMNSLRAEDTAVYYCARGLVSFRGYAF | |
| | DYWGQGTLVTVSS | |
| 8-A6_VL | DIQMTQSPSSLSASVGDRVTITCRASQSISSYLNWYQQKPGKAPKLLIYAASSL | 111 |
| | QSGVPSRFSGSGSGTDFTLTISSLQPEDFATYYCQQSYTIPPTFGQGTKVEIK | |
| 8-A7_VH | EVQLLESGGGLVQPGGSLRLSCAASGFTFRNYAMSWVRQAPGKGLEWVSSISGG | 112 |
| | GINTYYADSVKGRFTISRDNSKNTLYLQMNSLRAEDTAVYYCARVRSYSGPGYF | |
| | DYWGQGTLVTVSS | |
| 8-A7_VL | EIVMTQSPATLSVSPGDRATLSCRASRSVSSNLAWYQQKPGQAPRLLIYGASTR | 113 |
| | ATGIPARFSGSGSGTEFTLTISSLQSEDFAVYYCQQYYNWPLTFGQGTKVEIK | |
| 8-A8_VH | EVQLLESGGGLVQPGGSLRLSCAASGFTFSNYAMSWVRQAPGKGLEWVSVIYGS | 114 |
| | GARTYYADSVKGRFTISRDNSKNTLYLQMNSLRAEDTAVYYCARISPHYDFDVW | |
| | GQGTLVTVSS | |
| 8-A8_VL | DIQMTQSPSSLSASVGDRVTITCRASQTISKYLNWYQQKPGKAPKLLIYAASSL | 115 |
| | QSGVPSRFSGSGSGTDFTLTISSLQPEDFATYYCQQSYSYPITFGQGTKVEIK | |
| 8-A9_VH | EVQLLESGGGLVQPGGSLRLSCAASGFTFSNYAMSWVRQAPGKGLEWVSAISGG | 116 |
| | GSRTYYADSVKGRFTISRDNSKNTLYLQMNSLRAEDTAVYYCAREARVLGYSLD | |
| | YWGQGTLVTVSS | |
| 8-A9_VL | DIQMTQSPSSLSASVGDRVTITCRASQRIGKYLNWYQQKPGKAPKLLIYAASSL | 117 |
| | QSGVPSRFSGSGSGTDFTLTISSLQPEDFATYYCQQSYSYPLTFGQGTKVEIK | |
| 8-C4_VH | EVQLLESGGGLVQPGGSLRLSCAASGFTFSNYAMSWVRQAPGKGLEWVSAIYGS | 118 |
| | GGRTYYADSVKGRFTISRDNSKNTLYLQMNSLRAEDTAVYYCARLRYGFYQFDY | |
| | WGQGTLVTVSS | |
| 8-C4_VL | DIQMTQSPSSLSASVGDRVTITCRASQSIRNYLNWYEQKPGKAPKLLIYAASSL | 119 |
| | QSGVPSRFSGSGSGTDFTLTISSLQPEDFATYYCQQSYSYPLTFGQGTKVEIK | |
| 8-C6_VH | EVQLLESGGGLVQPGGSLRLSCAASGFTFTDYAMSWVRQAPGKGLEWVSTIYSS | 120 |
| | GHRTYYADSVKGRFTISRDNSKNTLYLQMNSLRAEDTAVYYCARLYPLHSYAFD | |
| | HWGQGTLVTVSS | |
| 8-C6_VL | DIQMTQSHSSLSASVGDRVTITCRASQSISRYLNWYQQKPGKAPKLLIYAASSL | 121 |
| | QSGVPSRFSGSGSGTDFTLTISSLQPEDFATYYCQQSYSVPLTFGQGTKVEIK | |
| 8-C9_VH | EVQLLESGGGLVQPGGSLRLSCAASGFTFKSYAMSWVRQAPGKGLEWVSLISSS | 122 |
| | GENTYYADSVKGRFTISRDNSKNTLYLQMNSLRAEDTAVYYCARVRFGYGSWRY | |
| | RKYMDVWGQGTLVTVSS | |
| 8-C9_VL | DIQMTQSPSSLSASVGDRVTITCRASQSIATYLNWYQQKPGKAPKLLIYGASSL | 123 |
| | QSGVPSRFSGSGSGTDFTLTISSLQPEDFATYYCQQSYSTPLTFGQGTKVEIK | |
| 8- | EVQLLESGGGLVQPGGSLRLSCAASGFTFSSYAMSWVRQAPGKGLEWVSTISAS | 124 |
| C10_VH | GATYYADSVKGRFTISRDNSKNTLYLQMNSLRAEDTAVYYCARGPYSYYYYSFD | |
| | YWGQGTLVTVSS | |
| 8- | DIQMTQSPSSLSASVGDRVTITCRASQTISTYLNWYQQKPGKAPKLLIYAASSL | 125 |
| C10_VL | QSGVPSRFSGSGSGTDFTLTISSLQPEDFATYYCQQSYRYPLTFGQGTKVEIK | |
| 8- | EVQLLESGGGLVQPGGSLRLSCAASGFTFSSYAMSWVRQAPGKGLEWVSTIYVG | 126 |
| C11_VH | GHRTYYADSVKGRFTISRDNSKNTLYLQMNSLRAEDTAVYYCARVGWSWGFDPW | |
| | GQGTLVTVSS | |
| 8- | DIQMTQSPSSLSASVGDRVTITCRASQTISKYLNWYQQKPGKAPKLLIYSASSL | 127 |
| C11_VL | QSGVPSRFSGSGSGTDFTLTISSLQPEDFATYYCQQSYTYPLTFGQGTKVEIK | |
| 8- | EVQLVESGGGLVKPGGSLRLSCAASGFTFTNAWMSWVRQAPGKGLEWVGRIKSI | 128 |
| D11_VH | TEGGTTDYAAPVKGRFTISRDDSKNTLYLQMNSLKTEDTAVYYCARVWHTDDYY | |
| | EGFDVWGQGTLVTVSS | |
| 8- | EIVLTQSPGTLSLSPGERATLSCRASQSVSGSYLAWYQQKPGQAPRLLIYDASS | 129 |
| D11_VL | RATGIPDRFSGSGSGTDFTLTISRLEPEDFAVYYCQQYGTTPQTFGQGTKVEIK | |
| 8-D4_VH | EVQLLESGGGLVQPGGSLRLSCAASGFTFRSYAMSWVRQAPGKGLEWVSTIYGS | 130 |
| | GARTYYADSVKGRFTISRDNSKNTLYLQMNSLRAEDTAVYYCARWGGDGFYAMD | |
| | YWGQGTLVTVSS | |
| 8-D4_VL | DIQMTQSPSSLSASVGDRVTITCRASQSIYTYLNWYQQKPGKAPKLLIYAASSL | 131 |
| | QSGVPSRFSGSGSGTDFTLTISSLQPEDFATYYCQQSYSFPLTFGQGTKVEIK | |
| 8-D7_VH | EVQLLESGGGLVQPGGSLRLSCAASGFTFSSYAMSWVRQAPGKGLEWVSGISTS | 132 |
| | GGTTYYADSVKGRFTISRDNSKNTLYLQMNSLRAEDTAVYYCARVVGRAVEDIW | |
| | GQGTLVTVSS | |
| 8-D7_VL | EIVLTQSPGTLSLSPGERATLSCRASQSVSSTYLAWYQQKPGQAPRLLIYAASS | 133 |
| | RATGIPDRFSGSGSGTDFTLTISRLEPEDFAVYYCQQYAALPITFGQGTKVEIK | |
| 8-E2_VH | QVQLVQSGAEVKKPGSSVKVSCKASGGTFSDEAIHWVRQAPGQGLEWMGRIIPV | 134 |
| | LGIASYAQKFQGRVTITADESTSTAYMELSSLRSEDTAVYYCARGLSRKAFYFD | |
| | YWGQGTLVTVSS | |
| 8-E2_VL | DIQMTQSPSSLSASVGDRVTITCRASQTIGNYLNWYQQKPGKAPKLLIYVASSL | 135 |
| | QSGVPSRFSGSGSGTDFTLTISSLQPEDFATYYCQQSYRYPYTFGQGTKVEIK | |
| 8-E3_VH | EVQLLESGGGLVQPGGSLRLSCAASGFTFSNYAMSWVRQAPGKGLEWVSTIYRG | 136 |
| | AGTYYADSVKGRFTISRDNSKNTLYLQMNSLRAEDTAVYYCARDASWYGPFSYY | |
| | YYYGFDVWGQGTLVTVSS | |
| 8-E3_VL | EIVMTQSPATLSVSPGERATLSCRASQSVYTNLAWYQQKPGQAPRLLIYDASTR | 137 |
| | ATGIPARFSGSGSGTEFTLTISSLQSEDFAVYYCQQYNDWPLTFGQGTKVEIK | |
| 8-E9_VH | EVQLLESGGGLVQPGGSLRLSCAASGFTFSDYAMSWVRQAPGKGLEWVSIITES | 138 |
| | GVNTYYADSVKGRFTISRDNSKNTLYLQMNSLRAEDTAVYYCARHPSVGSASRS | |
| | YYFDVWGQGTLVTVSS | |
| 8-E9_VL | EIVLTQSPGTLSLSPGERATLSCRASQSVGSSYLAWYQQKPGQAPRLLIYAASS | 139 |
| | RATGIPDRFSGSGSGTDFTLTISRLEPEDFAVYYCQQYYSAPLTFGQGTKVEIK | |
| 8-F1_VH | EVQLLESGGGLVQPGGSLRLSCAASGFTFTDYAMSWVRQAPGKGLEWVSGISGS | 140 |
| | GGVGTYYADSVKGRFTISRDNSKNTLYLQMNSLRAEDTAVYYCARGEYGSSIAY | |
| | SYYYGFDVWGQGTLVTVSS | |
| 8-F1_VL | EIVMTQSPATLSVSPGERATLSCRASQSVYNNLAWYQQKPGQAPRLLIYDASTR | 141 |
| | ATGIPARFSGSGSGTEFTLTISSLQSEDFAVYYCQQYNDWPYTFGQGTKVEIK | |
| 8-F2_VH | EVQLLESGGGLVQPGGSLRLSCAASGFTFSNYAMSWVRQAPGKGLEWVSAISGG | 142 |
| | GSRTYYADSVKGRFTISRDNSKNTLYLQMNSLRAEDTAVYYCAREARVLGYSLD | |
| | YWGQGTLVTVSS | |
| 8-F2_VL | DIQMTQSPSSLSASVGDRVTITCRASQRIGKYLNWYQQKPGKAPKLLIYAASSL | 143 |
| | QSGVPSRFSGSGSGTDFTLTISSLQPEDFATYYCQQSYSYPLTFGQGTKVEIK | |
| 8-F4_VH | EVQLLESGGGLVQPGGSLRLSCAASGFTFSGYAMSWVRQAPGKGLEWVSAIRGG | 144 |
| | GGTYYADSVKGRFTISRDNSKNTLYLQMNSLRAEDTAVYYCARVGPLRLYGFDY | |
| | WGQGTLVTVSS | |
| 8-F4_VL | DIQMTQSPSSLSASVGDRVTITCRASQSISNYLNWYQQKPGKAPKLLIYAASSL | 145 |
| | QSGVPSRFSGSGSGTDFTLTISSLQPEDFATYYCQQSYSYPLTFGQGTKVEIK | |
| 8-F6_VH | EVQLLESGGGLVQPGGSLRLSCAASGFTFSSYAMSWVRQAPGKGLEWVSVISGS | 146 |
| | GGNTYYADSVKGRFTISRDNSKNTLYLQMNSLRAEDTAVYYCAREVRHDSYYYY | |
| | YYSGMDVWGQGTLVTVSS | |
| 8-F6_VL | EIVMTQSPATLSVSPGERATLSCRASQSVYNNLAWYQQKPGQAPRLLIYDASTR | 147 |
| | ATGIPARFSGSGSGTEFTLTISSLQSEDFAVYYCQQYSDYPFTFGQGTKVEIK | |
| 8- | QVQLVQSGAEVKKPGSSVKVSCKASGGTFSNYAINWVRQAPGQGLEWMGRIIPT | 148 |
| F11_VH | LGIANYAQKFQGRVTITADESTSTAYMELSSLRSEDTAVYYCARAAYFAGPAAR | |
| | YGYFDIWGQGTLVTVSS | |
| 8- | EIVLTQSPGTLSLSPGERATLSCRASQSVRSNYLAWYQQKPGQAPRLLIYDASS | 149 |
| F11_VL | RATGIPDRFSGSGSGTDFTLTISRLEPEDFAVYYCQQYAYAPWTFGQGTKVEIK | |
| 8- | EVQLLESGGGLVQPGGSLRLSCAASGFTFRGYAMSWVRQAPGKGLEWVSSISVS | 150 |
| G12_VH | GAGTYYADSVKGRFTISRDNSKNTLYLQMNSLRAEDTAVYYCARYVRGVSFDVW | |
| | GQGTLVTVSS | |
| 8- | EIVLTQSPGTLSLSPGERATLSCRASQSVSSNYLAWYQQKPGQAPRLLIYGASS | 151 |
| G12_VL | RATGIPDRFSGSGSGTDFTLTISRLEPEDFAVYYCQQYGSSPAVTFGQGTKVEI | |
| | K | |
| 8-H7_VH | EVQLLESGGGLVQPGGSLRLSCAASGFTFSNYAMSWVRQAPGKGLEWVSAIYGS | 152 |
| | GGRTYYADSVKGRFTISRDNSKNTLYLQMNSLRAEDTAVYYCARLRYGFYQFDY | |
| | WGQGTLVTVSS | |
| 8-H7_VL | DIQMTQSPSSLSASVGDRVTITCRASQSIRNYLNWYEQKPGKAPKLLIYAASSL | 153 |
| | QSGVPSRFSGSGSGTDFTLTISSLQPEDFATYYCQQSYSYPLTFGQGTKVEIK | |
| 9-A6_VH | EVQLLESGGGLVQPGGSLRLSCAASGFTFSDYAMSWVRQAPGKGLEWVSTIYSG | 154 |
| | GVRTYYADSVKGRFTISRDNSKNTLYLQMNSLRAEDTAVYYCARGLVSFRGYAF | |
| | DYWGQGTLVTVSS | |
| 9-A6_VL | DIQMTQSPSSLSASVGDRVTITCRASQSISSYLNWYQQKPGKAPKLLIYAASSL | 155 |
| | QSGVPSRFSGSGSGTDFTLTISSLQPEDFATYYCQQSYTIPPTFGQGTKVEIK | |
| 9-B3_VH | QVQLVESGGGVVQPGRSLRLSCAASGFTFSDYGMHWVRQAPGKGLEWVAVISYD | 156 |
| | GSRKYYANSVKGRFTISRDNSKNTLYLQMNSLRAEDTAVYYCARGSYSDGFDVW | |
| | GQGTLVTVSS | |
| 9-B3_VL | DIQMTQSPSSLSASVGDRMTITCRASQSISNYLNWYQQKPGKAPKLLIYAASSL | 157 |
| | QSGVPSRFSGSGSGTDFTLTISSLQPEDFATYYCQQSYAPPYTFGQGTKVEIK | |
| 9- | EVQLLESGGGLVQPGGSLRLSCAASGFTFSSYAMSWVRQAPGKGLEWVSGISES | 158 |
| B10_VH | GGGTYYADSVKGRFTISRDNSKNTLYLQMNSLRAEDTAVYYCARLVPWQLAFDV | |
| | WGQGTLVTVSS | |
| 9- | EIVMTQSPATLSVSPGERATLSCRASQSVYNNLAWYQQKPGQAPRLLIYDASTR | 159 |
| B10_VL | ATGIPARFSGSGSGTEFTLTISSLQSEDFAVYYCQQYYYWPITFGQGTKVEIK | |
| 9-C2_VH | EVQLLESGGGLVQPGGSLRLSCAASGFTFSSYAMSWVRQAPGKGLEWVSVISGS | 160 |
| | GGNTYYADSVKGRFTISRDNSKNTLYLQMNSLRAEDTAVYYCAREVRHDSYYYY | |
| | YYSGMDVWGQGTLVTVSS | |
| 9-C2_VL | EIVMTQSPATLSVSPGERATLSCRASQSVYNNLAWYQQKPGQAPRLLIYDASTR | 161 |
| | ATGIPARFSGSGSGTEFTLTISSLQSEDFAVYYCQQYSDYPFTFGQGTKVEIK | |
| 9-D8_VH | QVQLVQSGAEVKKPGSSVKVSCKASGGTFSSEAISWVRQAPGQGLEWMGRIIPI | 162 |
| | SGRPNYAQKFQGRVTITADESTSTAYMELSSLRSEDTAVYYCAREASYYVAGYY | |
| | RYYGFDVWGQGTLVTVSS | |
| 9-D8_VL | EIVMTQSPATLSVSPGERATLSCRASRSLSNNLAWYQQKPGQAPRLLIYDASTR | 163 |
| | ATGIPARFSGSGSGTEFTLTISSLQSEDFAVYYCQQYNAWPYTFGQGTKVEIK | |
| 9-E4_VH | EVQLLESGGGLVQPGGSLRLSCAASGFTFSSYAMSWVRQAPGKGLEWVSVISGS | 164 |
| | GGNTYYADSVKGRFTISRDNSKNTLYLQMNSLRAEDTAVYYCAREVRHDSYYYY | |
| | YYSGMDVWGQGTLVTVSS | |
| 9-E4_VL | EIVMTQSPATLSVSPGERATLSCRASQSVYNNLAWYQQKPGQAPRLLIYDASTR | 165 |
| | ATGIPARFSGSGSGTEFTLTISSLQSEDFAVYYCQQYSDYPFTFGQGTKVEIK | |
| 9-E5_VH | QVQLVESGGGLVKPGGSLRLSCAASGFTFSNAWMSWVRQAPGKGLEWVGRIKSK | 166 |
| | IEGGTTDYAPPVKGRFTISRDDSKNTLYLQMNSLKTEDTAVYYCARDPTWYVSG | |
| | HYYGFDVWGQGTLVTVSS | |
| 9-E5_VL | DIQMTQSPSSLSASVGDRVTITCRASQSIANYLNWYQQKPGKAPKLLIYAASSL | 167 |
| | QSGVPSRFSGSGSGTDFTLTISSLQPEDFATYYCQQYFSTPPTFGQGTKVEIK | |
| 9-G1_VH | EVQLLESGGGLVQPGGSLRLSCAASGFTFGNYAMSWVRQAPGKGLEWVSTISAG | 168 |
| | GSRTYYADSVKGRFTISRDNSKNTLYLQMNSLRAEDTAVYYCARGWYPILYYAF | |
| | DYWGQGTLVTVSS | |
| 9-G1_VL | DIQMTQSPSSLSASVGDRVTITCRASQSIGNYLNWYQQKPGKAPKLLIYSASSL | 169 |
| | QSGVPSRFSGSGSGTDFTLTISSLQPEDFATYYCQQTYTIPLTFGQGTKVEIK | |
| 9-G3_VH | EVQLLESGGGLVQPGGSLRLSCAASGFTFSSYAMSWVRQAPGKGLEWVSTIYRS | 170 |
| | GGRTYYADSVKGRFTISRDNSKNTLYLQMNSLRAEDTAVYYCARGAYPLGSYYG | |
| | FDPWGQGTLVTVSS | |
| 9-G3_VL | DIQMTQSPSSLSASVGDRVTITCRASQSIATYLNWYQQKPGKAPKLLIYAASSL | 171 |
| | QSGVPSRFSGSGSGTDFTLTISSLQPEDFATYYCQQSYTYPVTFGQGTKVEIK | |
| 9-H9_VH | EVQLLESGGGLVQPGGSLRLSCAASGFTFSNYAMSWVRQAPGKGLEWVSTIYGS | 172 |
| | GVRTYYADGVKGRFTISRDNSKNTLYLQMNSLRAEDTAVYYCARGWIPHSFYAF | |
| | DYWGQGTLVTVSS | |
| 9-H9_VL | DIQMTQSPSSLSASVGDRVTITCRASQSISSYLNWYQQKPGKAPKLLIYGASSL | 173 |
| | QSGVPSRFSGSGSGTDFTLTISSLQPEDFATYYCQQSFTAPVTFGQGTKVEIK | |
| 2-C8_VH | EVQLLESGGGLVQPGGSLRLSCAASGFTFTNYAMSWVRQAPGKGLEWVSSISGS | 174 |
| | GISTYYADSVKGRFTISRDNSKNTLYLQMNSLRAEDTAVYYCARGPGPGYWYAF | |
| | DVWGQGTLVTVSS | |
| 2-C8_VL | DIQMTQSPSSLSASVGDRVTITCRASQTISRYLNWYQQKPGKAPKLLIYAASSL | 175 |
| | QSGVPSRFSGSGSGTDFTLTISSLQPEDFATYYCQQSYTYPITFGQGTKVEIK | |
| 8-D9_VH | QVQLVQSGAEVKKPGSSVKVSCKASGGTFSDEAIHWVRQAPGQGLEWMGRIIPV | 176 |
| | LGIASYAQKFQGRVTITADESTSTAYMELSSLRSEDTAVYYCARGLSRRAFYFD | |
| | YWGQGTLVTVSS | |
| 8-D9_VL | DIQMTQSPSSLSASVGDRVTITCRASQTIGNYLNWYQQKPGKAPKLLIYVASSL | 177 |
| | QSGVPSRFSGSGSGTDFTLTISSLQPEDFATYYCQQSYRYPYTFGQGTKVEIK | |
| 10- | EVQLLESGGGLVQPGGSLRLSCAASGFTFTNYAMSWVRQAPGKGLEWVSSISGS | 178 |
| A9_VH | GISTYYADSVKGRFTISRDNSKNTLYLQMNSLRAEDTAVYYCARGPGPGYWYAF | |
| | DVWGQGTLVTVSS | |
| 10- | DIQMTQSPSSLSASVGDRVTITCRASQTISRYLNWYQQKPGKAPKLLIYSASSL | 179 |
| A9_VL | QSGVPSRFSGSGSGTDFTLTISSLQPEDFATYYCQQSYTYPITFGQGTKVEIK | |
| 10- | EVQLLESGGGLVQPGGSLRLSCAASGFTFSDYAMSWVRQAPGKGLEWVSTIYSG | 180 |
| B2_VH | GVRTYYADSVKGRFTISRDNSKNTLYLQMNSLRAEDTAVYYCARGLVSFKGYAF | |
| | DYWGQGTLVTVSS | |
| 10- | DIQMTQSPSSLSASVGDRVTITCRASQSISSYLNWYQQKPGKAPKLLIYAASSL | 181 |
| B2_VL | QSGVPSRFSGSGSGTDFTLTISSLQPEDFATYYCQQSYTIPPTFGQGTKVEIK | |
| 10- | EVQLLESGGGLVQPGGSLRLSCAASGFTFSSYAMSWVRQAPGKGLEWVSAIYGA | 182 |
| B5_VH | GGKTYYADSVKGRFTISRDNSKNTLYLQMNSLRAEDTAVYYCARISHGWGFDVW | |
| | GQGTLVTVSS | |
| 10- | DIQMTQSPSSLSASVGDRVTITCRASQDISNYLNWYQQKPGKAPKLLIYAASSL | 183 |
| B5_VL | QSGVPSRFSGSGSGTDFTLTISSLQPEDFATYYCQQSYTYPLTFGQGTKVEIK | |
| 10- | EVQLLESGGGLVQPGGSLRLSCAASGFTFSNYAMSWVRQAPGKGLEWVSVIYGS | 184 |
| C1_VH | GTRTYYADSVKGRFTISRDNSKNTLYLQMNSLRAEDTAVYYCARVASSYHYAFD | |
| | YWGQGTLVTVSS | |
| 10- | DIQMTQSPSSLSASVGDRVTITCRASQSISTYLNWYQQKPGKAPKLLIYAASSL | 185 |
| C1_VL | QSGVSSRFSGSGSGTDFTLTISSLQPEDFATYYCQQSYTFPLTFGQGTKVEIK | |
| 10- | EVQLLESGGGLVQPGGSLRLSCAASGFTFSNYAMSWVRQAPGKGLEWVSVIYGS | 186 |
| C3_VH | GGRTYYADSVKGRFTISRDNSKNTLYLQMNSLRAEDTAVYYCARVEDTSYYGMD | |
| | YWGQGTLVTVSS | |
| 10- | DIQMTQSPSSLSASVGDRVTITCRASQTIGIYLNWYQQKPGKAPTLLIYAASSL | 187 |
| C3_VL | QSGVPSRFSGSGSGTDFTLTISSLQPEDFATYYCQQSYTYPLTFGQGTKVEIK | |
| 10- | EVQLLESGGGLVQPGGSLRLSCAASGFTFSNYAMSWVRQAPGKGLEWVSAIYGS | 188 |
| C9_VH | GGRTYYADSVKGRFTISRDNSKNTLYLQMNSLRAEDTAVYYCARLEAGFYSLDI | |
| | WGQGTLVTVSS | |
| 10- | DIQMTQSPSSLSASVGDRVTITCRASQTIGTYLNWYQQKPGKAPKLLIYAASSL | 189 |
| C9_VL | QSGVPSRFSGSGSGTDFTLTISSLQPEDFATYYCQQSYSYPLTFGQGTKVEIK | |
| 10- | EVQLLESGGGLVQPGGSLKLSCAASGFTFGSYAMSWVRQAPGKGLEWVSVISSG | 190 |
| C11_VH | GSYTYYADSVKGRFTISRDNSKNTLYLQMNSLRAEDTAVYYCARVSGVVGALDI | |
| | WGQGTLVTVSS | |
| 10- | EIVMTQSPATLSVSPGERATLSCRASQSVSNNLAWYQQKPGQAPRLLIYGASTR | 191 |
| C11_VL | ATGIPARFSGSGSGTEFTLTISSLQSEDFAVYYCQQYNNWPYTFGQGTKVEIK | |
| 10- | EVQLLESGGGLVQPGGSLRLSCAASGFTFSNYAMSWVRQAPGKGLEWVSTIYSS | 192 |
| D7_VH | GSRTYYADSVKGRFTISRDNSKNTLYLQMNSLRAEDTAVYYCARWGGDGFYAMD | |
| | YWGQGTLVTVSS | |
| 10- | DIQMTQSPSSLSASVGDRVTITCRASQSISTYLNWYQQKPGKAPKLLIYAASSL | 193 |
| D7_VL | QSGVPSRFSGSGSGTDFTLTISSLQPEDFATYYCQQSYTVPLTFGQGTKVEIK | |
| 10- | EVQLLESGGGLVQPGGSLRLSCAASGFTFSDYAMSWVRQAPGKGLEWVSTIYSG | 194 |
| E1_VH | GVRTYYADSVKGRFTISRDNSKNTLYLQMNSLRAEDTAVYYCARGLVSFKGYAF | |
| | DYWGQGTLVTVSS | |
| 10- | DIQMTQSPSSLSASVGDRVTITCRASQSISSYLNWYQQKPGKAPKLLIYAASSL | 195 |
| E1_VL | QSGVPSRFSGSGSGTDFTLTISSLQPEDFATYYCQQSYTIPPTFGQGTKVEIK | |
| 10- | EVQLLESGGGLVQPGGSLRLSCAASGFTFSSYAMSWVRQAPGKGLEWVSAIYGS | 196 |
| E6_VH | GGDTYYADSVKGRFTISRDNSKNTLYLQMNSLRAEDTAVYYCARTEYDGYFDVW | |
| | GQGTLVTVSS | |
| 10- | DIQMTQSPSSLSASVGDRVTITCRASQDIYNYLNWYQQKPGKAPKLLIYGASSL | 197 |
| E6_VL | QSGVPSRFSGSGSGTDFTLTISSLQPEDFATYYCQQSDGFPPTFGQGTKVEIK | |
| 10- | EVQLLESGGGLVQPGGSLRLSCAASGFTFTDYAMSWVRQAPGKGLEWVSVIYGS | 198 |
| E7_VH | GGRTYYADSVKGRFTISRDNSKNTLYLQMNSLRAEDTAVYYCARVEDSHYSFDV | |
| | WGQGTLVTVSS | |
| 10- | DIQMTQSPSSLSASVGDRVTITCRASQSISNYLNWYQQKPGKAPKLLIYAASSL | 199 |
| E7_VL | QSGVPSRFSGSGSGTDFTLTISSLQPEDFATYYCQQSYTVPWTFGQGTKVEIK | |
| 10- | QVQLVESGGGVVQPGRSLRLSCAASGFPFSDYAMHWLRQAPGKGLEWVAVISYD | 200 |
| F11_VH | GNIEYYANSVKGRFTISRDNSKNTLYLQMNSLRAEDTAVYYCARDRIGSSTYYG | |
| | YYNGFDVWGQGTLVTVSS | |
| 10- | EIVMTQSPATLSVSPGERATLSCRASQSVSSNLAWYQQKPGQAPRLLIYGASTR | 201 |
| F11_VL | ATGIPARFSGSGSGTEFTLTISSLQSEDFAVYYCQQYNDYPLTFGQGTKVEIK | |
| 10- | EVQLLESGGGLVQPGGSLRLSCAASGFTFSNYAMSWVRQAPGKGLEWVSAIKGS | 202 |
| F12_VH | GGGTYYADSVKGRFTISRDNSKNTLYLQMNSLRAEDTAVYYCARGGYLWLDPWG | |
| | QGTLVTVSS | |
| 10- | EIVMTQSPATLSVSPGERATLSCRASQSISSNLAWYQQKPGQAPRLLIYDASTR | 203 |
| F12_VL | ATGIPARFSGSGSGTEFTLTISSLQSEDFAVYYCQQYYNWPLTFGQGTKVEIK | |
| 10- | EVQLLESGGGLVQPGGSLRLSCAASGFTFSNYAMSWVRQAPGKGLEWVSAISGS | 204 |
| G5_VH | GTYTYYADSVKGRFTISRDNSKNTLYLQMNSLRAEDTAVYYCARVRISGYGPGY | |
| | FGGFDVWGQGTLVTVSS | |
| 10- | EIVMTQSPATLSVSPGERATLSCRASQSVYSNLAWYQQKPGQAPRLLIYGASTR | 205 |
| G5_VL | ATGIPARFSGSGSGTEFTLTISSLQSEDFAVYYCQQYSNWPLTFGQGTKVEIK | |
| 11- | EVQLLESGGGLVQPGGSLRLSCAASGFTFSRYAMSWVRQAPGKGLEWVSIISGT | 206 |
| B6_VH | GGNTYYADSVKGRFTISRDNSKNTLYLQMNSLRAEDTAVYYCARIDTFAYYYSL | |
| | YRAFDLWGQGTLVTVSS | |
| 11- | EIVMTQSPATLSVSPGERATLSCRASQSVGGNLAWYQQKPGQAPRLLIYDASTR | 207 |
| B6_VL | ATGIPARFSGSGSGTEFTLTISSLQSEDFAVYYCQQYNDWPLTFGQGTKVEIK | |
| 11- | EVQLLESGGGLVQPGGSLRLSCAASGFTFSDYAMSWVRQAPGKGLEWVSSISEG | 208 |
| D8_VH | GGSTYYADSVKGRFTISRDNSKNTLYLQMNSLRAEDTAVYYCARTKRYFAGGSY | |
| | YWMDVWGQGTLVTVSS | |
| 11- | DIQMTQSPSSLSASVGDRVTITCRASQRISSYLNWYQQKPGKAPKLLIYAASSL | 209 |
| D8_VL | QSGVPSRFSGSGSGTDFALTISSLQPEDFATYYCQQSYSYPITFGQGTKVEIK | |
| 11- | EVQLLESGGGLVQPGGSLRLSCAASGFTFSSYAMSWVRQAPGKGLEWVSVISGG | 210 |
| D10_VH | SGHTTYYADSVKGRFTISRDNSKNTLYLQMNSLRAEDTAVYYCARHGGGRFAMD | |
| | VWGQGTLVTVSS | |
| 11- | EIVLTQSPGTLSLSPGERATLSCRASQSVASPYLAWYQQKPGQAPRLLIYGASS | 211 |
| D10_VL | RATGIPDRFSGSGSGTDFTLTISRLEPEDFAVYYCQQYYESPITFGQGTKVEIK | |
| 11- | EVQLLESGGGLVQPGGSLRLSCAASGFTFSTYAMSWVRQAPGKGLEWVSSISGS | 212 |
| D11_VH | GGKTYYADSVKGRFTISRDNSKNTLYLQMNSLRAEDTAVYYCARDIYGITYDGY | |
| | TYYDGMDVWGQGTLVTVSS | |
| 11- | DIQMTQSPSSLSASVGDRVTITCRASQSIYNYLNWHQQKPGKAPKLLIYAASSL | 213 |
| D11_VL | QSGVPSRFSGSGSGTDFTLTISSLQPEDFATYYCQQSDSPPWTFGQGTKVEIK | |
| 11- | QVQLVQSGAEVKKPGSSVKVSCKASGGTFSNYYISWVRQAPGQGLEWMGGIIPV | 214 |
| G2_VH | FGVAHYAQKFQGRVTITADESTSTAYMELSSLRSEDTAVYYCARGYPGEYFDYW | |
| | GQGTLVTVSS | |
| 11- | EIVLTQSPGTLSLSPGERATLSCRASQSVSSSYLAWYQQKPGQAPRLLIYAASS | 215 |
| G2_VL | RATGIPDRFSGSGSGTDFTLTISRLEPEDFAVYYCQHYGGSPGYTFGQGTKVEI | |
| | K | |
| 11- | EVQLLESGGGLVQPGGSLRLSCAASGFTFSNYAMSWVRQAPGKGLEWVSTISGS | 216 |
| G5_VH | GANTYYADSVKGRFTISRDNSKNTLYLQMNSLRAEDTAVYYCARHHDIAPSWIY | |
| | YFDYWGQGTLVTVSS | |
| 11- | EIVLTQSPGTLSLSPGERATLSCRARQSVPSNYLAWYQQKPGQAPRLLIYGASS | 217 |
| G5_VL | RATGIPDRFSGSGSGTDFTLTISRLEPEDFAVYYCQQYYSSPLTFGQGTKVEIK | |
| 11- | EVQLLESGGGLVQPGGSLRLSCAASGFTFPDYAMSWVRQAPGKGLEWVSTIYAG | 218 |
| G9_VH | GGRTYYADSVKGRFTISRDNSKNTLYLQMNSLRAEDTAVYYCARGLTRHYGYSF | |
| | DHWGQGTLVTVSS | |
| 11- | DIQMTQSPSSLSASVGDRVTITCRASQTISSYLNWYQQKPGKAPKLLIYSASSL | 219 |
| G9_VL | QSGVPSRFSGSGSGTDFTLTISSLQPEDFATYYCQQSYTFPLTFGQGTKVEIK | |
| 11- | EVQLLESGGGLVQPGGSLRLSCAASGFTFSNYAMSWVRQAPGKGLEWVSSIYGS | 220 |
| H8_VH | GHRTYYADSVKGRFTISRDNSKNTLYLQMNSLRAEDTAVYYCARVASSAEYAFD | |
| | HWGQGTLVTVSS | |
| 11- | DIQMTQSPSSLSASVGDRVTITCRASQSISNYLNWYQQKSGKAPKLLIYAASSL | 221 |
| H8_VL | QSGVPSRFSGSGSGTDFTLTISSLQPEDFATYYCQQSYTVPLTFGQGTKVEIK | |
| 11- | EVQLLESGGGLVQPGGSLRLSCAASGFTFKNYAMSWVRQAPGKGLEWVSGISEG | 222 |
| H11_VH | GANTYYADSVKGRFTISRDNSKNTLYLQMNSLRAEDTAVYYCAREPPKYGAYSY | |
| | YGYGFDPWGQGTLVTVSS | |
| 11- | EIVLTQSPGTLSLSPGERATLSCRASQSVSSSYLAWYQQKPGQAPRLLIYGASS | 223 |
| H11_VL | RATGIPDRFSGSGSGTDFTLTISRLEPEDFAVYYCQQYSSSPTYTFGQGTKVEI | |
| | K | |

### Example 3. Killing of Tumor Endothelial Cells by PLXDC-Activating Antibodies

This example tested some of the identified antibodies for their activity in activating PLXDC signaling.

It was first shown that antibodies 3-G7 and 8-C9 activated the promotor activity in PLXDC1-expressing cells, but not in cells without PLXDC1.

The killing of human tumor endothelial cells was also tested ex vivo. With the ex vivo model of tumor angiogenesis demonstrated in Example 1, antibodies were added after tumor endothelial cells grew out of the tumor. Incubation of human lung tumor endothelial cells with control IgG (500 nM) did not lead to the death of tumor endothelial cells expressing PLXDC1. Incubation of human lung tumor endothelial cells with antibody 3-G7 (500 nM) led to the death of tumor endothelial cells expressing PLXDC1, as evident by comparing the tumor endothelial cell signal between day 1 and day 8. Incubation of human lung tumor endothelial cells with 8-C9 also led to the death of tumor endothelial cells expressing PLXDC1, as evident by comparing the tumor endothelial cell signal between day 1 and day 8.

### Example 4. Analysis of Domain A Functions

This example analyzed the function of Domain A of PLXDC1.

First, this example generated PLXDC1 knockout mice and found that these knockout mice, in contrast to wild-type mice, no longer respond that antibodies that target PLXDC1. Specifically, antibodies that target PLXDC1 killed tumor blood vessels expressing PLXDC1 to lead to tumor killing, but the same tumor model in a PLXDC1 knockout mice did not respond to the antibodies (tumor blood vessels and tumors were not killed).

Next, a PLXDC1 mutant with Domain A deleted was tested in cells. Wild-type PLXDC1 and a mutant version with Domain A deleted (PLXDC1-dA) were expressed in adherent and non-adherent cells. In adherent cells, expression of PLXDC1 did not cause cell death (FIG. 2). By contrast, expression of Domain A-deleted PLXDC1 (PLXDC1-dA) led to pronounced cell death. In nonadherent cells, neither the expression of PLXDC1 nor PLXDC1-dA caused cell death (FIG. 2). Therefore, this result shows that in adherent, but not in nonadherent cells, PLXDC1-dA triggered significant cell death, leading to greatly reduced cell number.

This assay, therefore, demonstrates that removal of Domain A can trigger cell death in a ligand-independent manner. Domain A deletion leads to receptor activation and triggers downstream cell death pathway. This pathway applies to adherent cells such as endothelial cells but not to nonadherent cells such as blood cells. This demonstrates domain A's function as an inhibitory domain for receptor activation.

Given that Domain A was identified as an inhibitory domain of the PLXDC protein, the antibodies of Table 3 were tested for their ability to bind Domain A. At least ten of them were able to bind PLXDC1 but not the mutant with Domain A deleted. The Domain A-binding antibodies are 3-C11, 4-A8, 5-E2, 2-H2, 3-G6, 3-G7, 8-A7, 8-A9, 8-C9 and 8-D9.

### Example 5. Generation of Domain A-Targeted Antibodies

This example screened for additional antibodies that specifically bind to Domain A and are capable of activating PLXDC signaling.

Using the antibody screening strategy described in Example 2, this example further identified 21 top antibodies that activated PLXDC1 by binding to domain A of PLXDC1. Along with the 10 Domain A-binding antibodies from Table 3, all of the 31 Domain A-binding antibodies are listed in Table 4 below, with all of their CDR sequences listed in Table 5.

TABLE 4
Domain A-Binding PLXDC1-Activating Antibodies

| | | SEQ ID |
|---|---|---|
| Name | Sequence (or name of antibody from Table 3) | NO: |
| A001 VH | 3-C11_VH | 38 |
| A001 VL | 3-C11_VL | 39 |
| A002 VH | 4-A8_VH | 82 |
| A002 VL | 4-A8_VL | 83 |
| A003 VH | 5-E2_VH | 98 |
| A003 VL | 5-E2_VL | 99 |
| A004 VH | 2-H2_VH | 20 |
| A004 VL | 2-H2_VL | 21 |
| A005 VH | 3-G6_VH | 70 |
| A005 VL | 3-G6_VL | 71 |
| A006 VH | 3-G7_VH | 72 |
| A006 VL | 3-G7_VL | 73 |
| A007 VH | 8-A7_VH | 112 |
| A007 VL | 8-A7_VL | 113 |
| A008 VH | 8-A9_VH | 116 |
| A008 VL | 8-A9_VL | 117 |
| A009 VH | 8-C9_VH | 122 |
| A009 VL | 8-C9_VL | 123 |
| A010 VH | 8-D9_VH | 176 |
| A010 VL | 8-D9_VL | 177 |
| A011 VH | QVQLVESGGGVVQPGRSLRLSCAASGFSFSDYGMHWVRQAPGKGLEWVAVISYE | 224 |
| | GSNEYYADSVKGRFTISRDNSKNTLYLQMNSLRAEDTAVYYCARGSEGSGLDVW | |
| | GQGTLVTVSS | |
| A011 VL | DIQMTQSPSSLSASVGDRVTITCRASQRIYNYLNWYQQKPGKAPKLLIYAASSL | 225 |
| | QSGVPSRFSGSSSGTDFTLTISSLQPEDFATYYCQQAYSPPYTFGQGTKVEIK | |
| A012 VH | EVQLLESGGGLVQPGGSLRLSCAASGFTFSNYAMSWVRQAPGKGLEWVSAISGG | 226 |
| | GSRTFYADSVKGRFTISRDNSKNTLYLQMDSLRAEDTAVYYCAREARVLGYSED | |
| | YWGQGTLVTVSS | |
| A012 VL | DIQMTQSPSSLSASVGDRVTITCRASQRIGTYLNWYQQKPGKAPKLLIYAASSL | 227 |
| | QSGVPSRFSGSGSGTDFTLTISSLQPEDFATYYCQQSYSYPLTFGQGTKVEIK | |
| A013 VH | QVQLVESGGGVVQPGRSLRLSCAASGFTFSDYGMHWVRQAPGKGLEWVAVISYE | 228 |
| | GSNEYYADSVKGRFTISRDNSKNTLYLQMNSLRAEDTAVYYCARGSDGDGFDVW | |
| | GQGTLVTVSS | |
| A013 VL | DIQMTQSPSSLSASVGDRVTITCRASQRIANYLNWYQQKPGKAPKLLIYAASSL | 229 |
| | QSGVPSRFSGSGSGTDFTLTISSLQPEDFATYYCQQSYSPPYTFGQGTKVEIK | |
| A014 VH | QVQLVESGGGVVQPGRSLRLSCAASGFTFSDYGMHWVRQAPGKGLEWVAVISYE | 230 |
| | GSREYYADSVKGRFTISRDNSKNTLYLQMNSLRAEDTAVYYCARGSEGDGFDVW | |
| | GQGTLVTVSS | |
| A014 VL | DIQMTQSPSSLSASVGDRVTITCRASQRISNYLNWYQQKPGKAPKLLIYAASSL | 231 |
| | QSGVPSRFSGSGSGTDFTLTISSLQPEDFATYYCQQSYSAPYTFGQGTKVEIK | |
| A015 VH | QVQLVESGGGVVQPGRSLRLSCAASGFTEDDYGMHWVRQAPGKGLEWVAVISYE | 232 |
| | GSNQYYADSVKGRFTISRDNSKNTLYLQMNSLRAEDTAVYYCARGSDGSGFDVW | |
| | GQGTLVTVSS | |
| A015 VL | DIQMTQSPSSLSASVGDRVTITCRASQDISNYLNWYQQKPGKAPKLLIYAASSL | 233 |
| | QSGVPSRFSGSGSGTDFTLTISSLQPEDFATYYCQQAYSPPYTFGQGTKVEIK | |
| A016 VH | QVQLVESGGGVVQPGRSLRLSCAASGFAFSDYGMHWVRQAPGKGLEWVAVISYE | 234 |
| | GSREYYADSVKGRFTISRDNSKNTLYLQMNSLRAEDTAVYYCARGSEGDGFDVW | |
| | GQGTLVTVSS | |
| A016 VL | DIQMTQSPSSLSASVGDRVTITCRASQRIYNYLNWYQQKPGKAPKLLIYAASSL | 235 |
| | QSGVPSRFSGSGSGTDFTLTISSLQPEDFATYYCQQSYSPPYTFGQGTKVEIK | |
| A017 VH | QVQLVESGGGVVQPGRSLRLSCAASGFAFSDYGMHWVRQAPGKGLEWVAVISYE | 236 |
| | GNNEYYADSVKGRFTISRDNSKNTLYLQMNSLRAEDTAVYYCARGSDGDGFDVW | |
| | GQGTLVTVSS | |
| A017 VL | DIQMTQSPSSLSASVGDRVTITCRASQRIGNYLNWYQQKPGKAPKLLIYAASSL | 237 |
| | QSGVPSRFSGSGSGTDFTLTISSLQPEDFATYYCQQSYSPPYTFGQGTKVEIK | |
| A018 VH | EVQLLESGGGLVQPGGSLRLSCAASGFTFSAYAMSWVRQAPGKGLEWVSTISGG | 238 |
| | GSRTFYADSVKGRFTISRDNSKNTLYLQMNSLRAEDTAVYYCAREAVVLGYSLD | |
| | YWGQGTLVTVSS | |
| A018 VL | DIQMTQSPSSLSASVGDRVTITCRASQGIGKYLNWYQQKPGKAPKLLIYAASSL | 239 |
| | QSGVPSRFSGSGSGTDFTLTISSLQPEDFATYYCQQSYSYPLTFGQGTKVEIK | |
| A019 VH | EVQLLESGGGLVQPGGSLRLSCAASGFTFSNYAMSWVRQAPGKGLEWVSIISGG | 240 |
| | GSRTYYADSVKGRFTISRDNSKNTLYLQMNSLRAEDTAVYYCAREARVLGYSLD | |
| | YWGQGTLVTVSS | |
| A019 VL | DIQMTQSPSSLSASVGDRVTITCRASQRISKYLNWYQQKPGKAPKLLIYAASSL | 241 |
| | QSGVPSRFSGSGSGTDFTLTISSLQPEDFATYYCQQSFSYPLTFGQGTKVEIK | |
| A020 VH | EVQLLESGGGLVQPGGSLRLSCAASGFTFSGYAMSWVRQAPGKGLEWVSIISGG | 242 |
| | GSRTYYADSVKGRFTISRDNSKNTLYLQMNSLRAEDTAVYYCAREARVLGYSLD | |
| | YWGQGTLVTVSS | |
| A020 VL | DIQMTQSPSSLSASVGDRVTITCRASQRISKYLNWYQQKPGKAPKLLIYAASSL | 243 |
| | QSGVPSRFSGSGSGTDFTLTISSLQPEDFATYYCQQSYIYPLTFGQGTKVEIK | |
| A021 VH | EVQLLESGGGLVQPGGSLRLSCAASGFTFSTYAMSWVRQAPGKGLEWVSIISGG | 244 |
| | GSRTYYADSVKGRFTVSRDNSKNTLYLQMNSLRAEDTAVYYCAREHRVLGYSLD | |
| | YWGQGTLVTVSS | |
| A021 VL | DIQMTQSPSSLSASVGDRVTITCRASQRISKYLNWYQQKPGKAPKLLIYAASSL | 245 |
| | QSGVPSRFSGSGSGTDFTLTISSLQPEDFATYYCQQSYIYPLTFGQGTKVEIK | |
| A022 VH | EVQLLESGGGLVQPGGSLRLSCAASGFTFGNYAMSWVRQAPGKGLEWVSIISGG | 246 |
| | GSRTYYADSVKGRFTISRDNSKNTLYLQMNSLRAEDTAVYYCAREARVLGYSLD | |
| | YWGQGTLVTVSS | |
| A022 VL | DIQMTQSPSSLSASVGDRVTITCRASQRISKYLNWYQQKPGKAPKLLIYAASSL | 247 |
| | QSGVPSRFSGSGSGTDFTLTISSLQPEDFATYYCQQSYIYPLTFGQGTKVEIK | |
| A023 VH | EVQLLESGGGLVQPGGSLRLSCAASGFTFGNYAMSWVRQAPGKGLEWVSAISGG | 248 |
| | GSRTFYADSVKGRFTISRDNSKNTLYLQMNSLRAEDTAVYYCAREARVLFYSLD | |
| | YWGQGTLVTVSS | |
| A023 VL | DIQMTQSPSSLSASVGDRVTITCRASQRIGIYLNWYQQKPGKAPKLLIYAASSL | 249 |
| | QSGVPSRFSGSGSGTDFTLTISSLQPEDFATYYCQQSYSYPLTFGQGTKVEIK | |
| A024 VH | QVQLVESGGGVVQPGRSLRLSCAASGFTFSDYGMHWVRQAPGKGLEWVAVISYE | 250 |
| | GSREYYADSVKGRFTISRDNSKNTLYLQMNSLRAEDTAVYYCARGSEGDGFDVW | |
| | GQGTLVTVSS | |
| A024 VL | DIQMTQSPSSLSASVGDRVTITCRASQRIYNYLNWYQQKPGKAPKLLIYAASSL | 251 |
| | QSGVPSRFSGSGSGTDFTLTISSLQPEDFATYYCQQGYSPPYTFGQGTKVEIK | |
| A025 VH | QVQLVESGGGVVQPGRSLRLSCAASGFIFSDYGMHWVRQAPGKGLEWVAVISYE | 252 |
| | GSREYYADSVKGRFTISRDNSKNTLYLQMNSLRAEDTAVYYCARGSEGDGFDVW | |
| | GQGTLVTVSS | |
| A025 VL | DIQMTQSPSSLSASVGDRVTITCRASQRISSYLNWYQQKPGKAPKLLIYAASSL | 253 |
| | QSGVPSRFSGSGSGTDFTLTISSLQPEDFATYYCQQSYSAPYTFGQGTKVEIK | |
| A026 VH | QVQLVESGGGVVQPGRSLRLSCAASGFTFDDYGMHWVRQAPGKGLEWVAVISYE | 254 |
| | GSNQYYADSVKGRFTISRDNSKNTLYLQMNSLRAEDTAVYYCARGSEGDGEDVW | |
| | GQGTLVTVSS | |
| A026 VL | DIQMTQSPSSLSASVGDRVTITCRASQRIGNYLNWYQQKPGKAPKLLIYAASSL | 255 |
| | QSGVPSRFSGSGSGTDFTLTISSLQPEDFATYYCQQGYSPPYTFGQGTKVEIK | |
| A027 VH | QVQLVESGGGVVQPGRSLRLSCAASGFIFSDYGMHWVRQAPGKGLEWVAVISYE | 256 |
| | GSREYYADSVKGRFTISRDNSKNTLYLQMNSLRAEDTAVYYCARGSEGDGFDVW | |
| | GQGTLVTVSS | |
| A027 VL | DIQMTQSPSSLSASVGDRVTITCRASQRISNYLNWYQQKPGKAPKLLIYAASSL | 257 |
| | QSGVPSRFSGSGSGTDFTLTISSLQPEDFATYYCQQGYSPPYTFGQGTKVEIK | |
| A028 VH | QVQLVESGGGVVQPGRSLRLSCAASGFIFSDYGMHWVRQAPGKGLEWVAVISYE | 258 |
| | GSREYYADSVKGRFTISRDNSKNTLYLQMNSLRAEDTAVYYCARGSEGDGFDVW | |
| | GQGTLVTVSS | |
| A028 VL | DIQMTQSPSSLSASVGDRVTITCRASQRISKYLNWYQQKPGKAPKLLIYAASSL | 259 |
| | QSGVPSRFSGSGSGTDFTLTISSLQPEDFATYYCQQSYSPPYTFGQGTKVEIK | |
| A029 VH | QVQLVESGGGVVQPGRSLRLSCAASGFTFGDYGMHWVRQAPGKGLEWVAVISYE | 260 |
| | GSREYYADSVKGRFTISRDNSKNTLYLQMNSLRAEDTAVYYCARGSEGDGFDVW | |
| | GQGTLVTVSS | |
| A029 VL | DIQMTQSPSSLSASVGDRVTITCRASQRIYNYLNWYQQKPGKAPKLLIYAASSL | 261 |
| | QSGVPSRFSGSGSGTDFTLTISSLQPEDFATYYCQQSYSPPYTFGQGTKVEIK | |
| A030 VH | QVQLVESGGGVVQPGRSLRLSCAASGFIFSDYGMHWVRQAPGKGLEWVAVISYE | 262 |
| | GSREYYADSVKGRFTISRDNSKNTLYLQMNSLRAEDTAVYYCARGSEGDGFDVW | |
| | GQGTLVTVSS | |
| A030 VL | DIQMTQSPSSLSASVGDRVTITCRASQTISNYLNWYQQKPGKAPKLLIYAASSL | 263 |
| | QSGVPSRFSGSGSGTDFTLTISSLQPEDFATYYCQQSYSPPYTFGQGTKVEIK | |
| A031 VH | EVQLLESGGGLVQPGGSLRLSCAASGFTFSNYAMSWVRQAPGKGLEWVSAISGG | 264 |
| | GSYTYYADSVKGRFTISRDNSKNTLYLQMNSLRAEDTAVYYCAREERVLGYSLD | |
| | YWGQGTLVTVSS | |
| A031 VL | DIQMTQSPSSLSASVGDRVTITCRASQRIYKYLNWYQQKPGKAPKLLIYAASSL | 265 |
| | QSGVPSRFSGSGSGTDFTLTISSLQPEDFATYYCQQSFSYPLTFGQGTKVEIK | |

TABLE 5
CDR Sequences

| Antibody | Domain | Sequence | SEQ ID NO: |
|---|---|---|---|
| A001 | VH CDR1 | SYAMS | 266 |
| | VH CDR2 | TIYASGATTYYADSVKG | 267 |
| | VH CDR3 | LKLYHAAFDI | 268 |
| | VL CDR1 | RASQDISSYLN | 269 |
| | VL CDR2 | TASSLQS | 270 |
| | VL CDR3 | QQSYSYPLT | 271 |
| A002 | VH CDR1 | NYAMS | 272 |
| | VH CDR2 | AISASGGYTYYADSVKG | 273 |
| | VH CDR3 | GRWGAFDY | 274 |
| | VL CDR1 | TGSSSNIGSGYDVH | 275 |
| | VL CDR2 | GNRNRPS | 276 |
| | VL CDR3 | QSYDSSLKTWV | 277 |
| A003 | VH CDR1 | DYGMH | 278 |
| | VH CDR2 | VISYHGRNEYYANSVKG | 279 |
| | VH CDR3 | GAYGDDFDV | 280 |
| | VL CDR1 | RASQSISSYLN | 281 |
| | VL CDR2 | AASSLQS | 282 |
| | VL CDR3 | QQSYSAPYT | 283 |
| A004 | VH CDR1 | DYGMH | 284 |
| | VH CDR2 | VISYEGSNEYYADSVKG | 285 |
| | VH CDR3 | GSEGSGFDV | 286 |
| | VL CDR1 | RASQRISNYLN | 287 |
| | VL CDR2 | AASSLQS | 288 |
| | VL CDR3 | QQSYSPPYT | 289 |
| A005 | VH CDR1 | DYGMH | 290 |
| | VH CDR2 | VISYDGSRKYYANSVKG | 291 |
| | VH CDR3 | GSYSDGFDV | 292 |
| | VL CDR1 | RASQSISRYLN | 293 |
| | VL CDR2 | GASSLQS | 294 |
| | VL CDR3 | QQSYSTPYT | 295 |
| A006 | VH CDR1 | TEYIS | 296 |
| | VH CDR2 | RIIPVLGITNYAQKFQG | 297 |
| | VH CDR3 | HLGPPYLVTYSHGFDV | 298 |
| | VL CDR1 | RASQSLGTNLA | 299 |
| | VL CDR2 | GASTRAT | 300 |
| | VL CDR3 | QQYYEAPPT | 301 |
| A007 | VH CDR1 | NYAMS | 302 |
| | VH CDR2 | SISGGGINTYYADSVKG | 303 |
| | VH CDR3 | VRSYSGPGYFDY | 304 |
| | VL CDR1 | RASRSVSSNLA | 305 |
| | VL CDR2 | GASTRAT | 306 |
| | VL CDR3 | QQYYNWPLT | 307 |
| A008 | VH CDR1 | NYAMS | 308 |
| | VH CDR2 | AISGGGSRTYYADSVKG | 309 |
| | VH CDR3 | EARVLGYSLDY | 310 |
| | VL CDR1 | RASQRIGKYLN | 311 |
| | VL CDR2 | AASSLQS | 312 |
| | VL CDR3 | QQSYSYPLT | 313 |
| A009 | VH CDR1 | SYAMS | 314 |
| | VH CDR2 | LISSSGENTYYADSVKG | 315 |
| | VH CDR3 | VRFGYGSWRYRKYMDV | 316 |
| | VL CDR1 | RASQSIATYLN | 317 |
| | VL CDR2 | GASSLQS | 318 |
| | VL CDR3 | QQSYSTPLT | 319 |
| A010 | VH CDR1 | DEAIH | 320 |
| | VH CDR2 | RIIPVLGIASYAQKFQG | 321 |
| | VH CDR3 | GLSRRAFYFDY | 322 |
| | VL CDR1 | RASQTIGNYLN | 323 |
| | VL CDR2 | VASSLQS | 324 |
| | VL CDR3 | QQSYRYPYT | 325 |
| A011 | VH CDR1 | DYGMH | 326 |
| | VH CDR2 | VISYEGSNEYYADSVKG | 327 |
| | VH CDR3 | GSEGSGLDV | 328 |
| | VL CDR1 | RASQRIYNYLN | 329 |
| | VL CDR2 | AASSLQS | 330 |
| | VL CDR3 | QQAYSPPYT | 331 |
| A012 | VH CDR1 | NYAMS | 332 |
| | VH CDR2 | AISGGGSRTFYADSVKG | 333 |
| | VH CDR3 | EARVLGYSFDY | 334 |
| | VL CDR1 | RASQRIGTYLN | 335 |
| | VL CDR2 | AASSLQS | 336 |
| | VL CDR3 | QQSYSYPLT | 337 |
| A013 | VH CDR1 | DYGMH | 338 |
| | VH CDR2 | VISYEGSNEYYADSVKG | 339 |
| | VH CDR3 | GSDGDGFDV | 340 |
| | VL CDR1 | RASQRIANYLN | 341 |
| | VL CDR2 | AASSLQS | 342 |
| | VL CDR3 | QQSYSPPYT | 343 |
| A014 | VH CDR1 | DYGMH | 344 |
| | VH CDR2 | VISYEGSREYYADSVKG | 345 |
| | VH CDR3 | GSEGDGFDV | 346 |
| | VL CDR1 | RASQRISNYLN | 347 |
| | VL CDR2 | AASSLQS | 348 |
| | VL CDR3 | QQSYSAPYT | 349 |
| A015 | VH CDR1 | DYGMH | 350 |
| | VH CDR2 | VISYEGSNQYYADSVKG | 351 |
| | VH CDR3 | GSDGSGFDV | 352 |
| | VL CDR1 | RASQDISNYLN | 353 |
| | VL CDR2 | AASSLQS | 354 |
| | VL CDR3 | QQAYSPPYT | 355 |
| A016 | VH CDR1 | DYGMH | 356 |
| | VH CDR2 | VISYEGSREYYADSVKG | 357 |
| | VH CDR3 | GSEGDGFDV | 358 |
| | VL CDR1 | RASQRIYNYLN | 359 |
| | VL CDR2 | AASSLQS | 360 |
| | VL CDR3 | QQSYSPPYT | 361 |
| A017 | VH CDR1 | DYGMH | 362 |
| | VH CDR2 | VISYEGNNEYYADSVKG | 363 |
| | VH CDR3 | GSDGDGFDV | 364 |
| | VL CDR1 | RASQRIGNYLN | 365 |
| | VL CDR2 | AASSLQS | 366 |
| | VL CDR3 | QQSYSPPYT | 367 |
| A018 | VH CDR1 | AYAMS | 368 |
| | VH CDR2 | TISGGGSRTFYADSVKG | 369 |
| | VH CDR3 | EAVVLGYSLDY | 370 |
| | VL CDR1 | RASQGIGKYLN | 371 |
| | VL CDR2 | AASSLQS | 372 |
| | VL CDR3 | QQSYSYPLT | 373 |
| A019 | VH CDR1 | NYAMS | 374 |
| | VH CDR2 | IISGGGSRTYYADSVKG | 375 |
| | VH CDR3 | EARVLGYSLDY | 376 |
| | VL CDR1 | RASQRISKYLN | 377 |
| | VL CDR2 | AASSLQS | 378 |
| | VL CDR3 | QQSFSYPLT | 379 |
| A020 | VH CDR1 | GYAMS | 380 |
| | VH CDR2 | IISGGGSRTYYADSVKG | 381 |
| | VH CDR3 | EARVLGYSLDY | 382 |
| | VL CDR1 | RASQRISKYLN | 383 |
| | VL CDR2 | AASSLQS | 384 |
| | VL CDR3 | QQSYIYPLT | 385 |
| A021 | VH CDR1 | TYAMS | 386 |
| | VH CDR2 | IISGGGSRTYYADSVKG | 387 |
| | VH CDR3 | EHRVLGYSLDY | 388 |
| | VL CDR1 | RASQRISKYLN | 389 |
| | VL CDR2 | AASSLQS | 390 |
| | VL CDR3 | QQSYIYPLT | 391 |
| A022 | VH CDR1 | NYAMS | 392 |
| | VH CDR2 | IISGGGSRTYYADSVKG | 393 |
| | VH CDR3 | EARVLGYSLDY | 394 |
| | VL CDR1 | RASQRISKYLN | 395 |
| | VL CDR2 | AASSLQS | 396 |
| | VL CDR3 | QQSYIYPLT | 397 |
| A023 | VH CDR1 | NYAMS | 398 |
| | VH CDR2 | AISGGGSRTFYADSVKG | 399 |
| | VH CDR3 | EARVLFYSLDY | 400 |
| | VL CDR1 | RASQRIGIYLN | 401 |
| | VL CDR2 | AASSLQS | 402 |
| | VL CDR3 | QQSYSYPLT | 403 |
| A024 | VH CDR1 | DYGMH | 404 |
| | VH CDR2 | VISYEGSREYYADSVKG | 405 |
| | VH CDR3 | GSEGDGFDV | 406 |
| | VL CDR1 | RASQRIYNYLN | 407 |
| | VL CDR2 | AASSLQS | 408 |
| | VL CDR3 | QQGYSPPYT | 409 |
| A025 | VH CDR1 | DYGMH | 410 |
| | VH CDR2 | VISYEGSREYYADSVKG | 411 |
| | VH CDR3 | GSEGDGFDV | 412 |
| | VL CDR1 | RASQRISSYLN | 413 |
| | VL CDR2 | AASSLQS | 414 |
| | VL CDR3 | QQSYSAPYT | 415 |
| A026 | VH CDR1 | DYGMH | 416 |
| | VH CDR2 | VISYEGSNQYYADSVKG | 417 |
| | VH CDR3 | GSEGDGFDV | 418 |
| | VL CDR1 | RASQRIGNYLN | 419 |
| | VL CDR2 | AASSLQS | 420 |
| | VL CDR3 | QQGYSPPYT | 421 |
| A027 | VH CDR1 | DYGMH | 422 |
| | VH CDR2 | VISYEGSREYYADSVKG | 423 |
| | VH CDR3 | GSEGDGFDV | 424 |
| | VL CDR1 | RASQRISNYLN | 425 |
| | VL CDR2 | AASSLQS | 426 |
| | VL CDR3 | QQGYSPPYT | 427 |
| A028 | VH CDR1 | DYGMH | 428 |
| | VH CDR2 | VISYEGSREYYADSVKG | 429 |
| | VH CDR3 | GSEGDGFDV | 430 |
| | VL CDR1 | RASQRISKYLN | 431 |
| | VL CDR2 | AASSLQS | 432 |
| | VL CDR3 | QQSYSPPYT | 433 |
| A029 | VH CDR1 | DYGMH | 434 |
| | VH CDR2 | VISYEGSREYYADSVKG | 435 |
| | VH CDR3 | GSEGDGFDV | 436 |
| | VL CDR1 | RASQRIYNYLN | 437 |
| | VL CDR2 | AASSLQS | 438 |
| | VL CDR3 | QQSYSPPYT | 439 |
| A030 | VH CDR1 | DYGMH | 440 |
| | VH CDR2 | VISYEGSREYYADSVKG | 441 |
| | VH CDR3 | GSEGDGFDV | 442 |
| | VL CDR1 | RASQTISNYLN | 443 |
| | VL CDR2 | AASSLQS | 444 |
| | VL CDR3 | QQSYSPPYT | 445 |
| A031 | VH CDR1 | NYAMS | 446 |
| | VH CDR2 | AISGGGSYTYYADSVKG | 447 |
| | VH CDR3 | EERVLGYSLDY | 448 |
| | VL CDR1 | RASQRIYKYLN | 449 |
| | VL CDR2 | AASSLQS | 450 |
| | VL CDR3 | QQSFSYPLT | 451 |

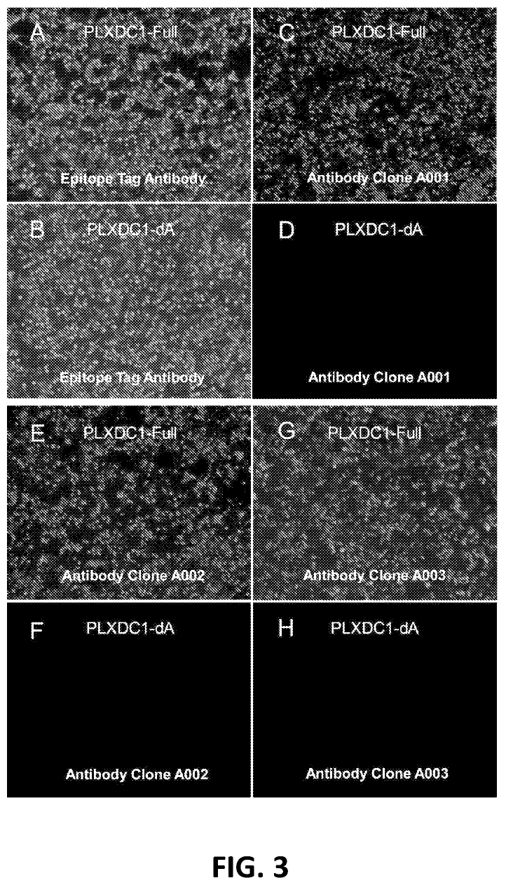

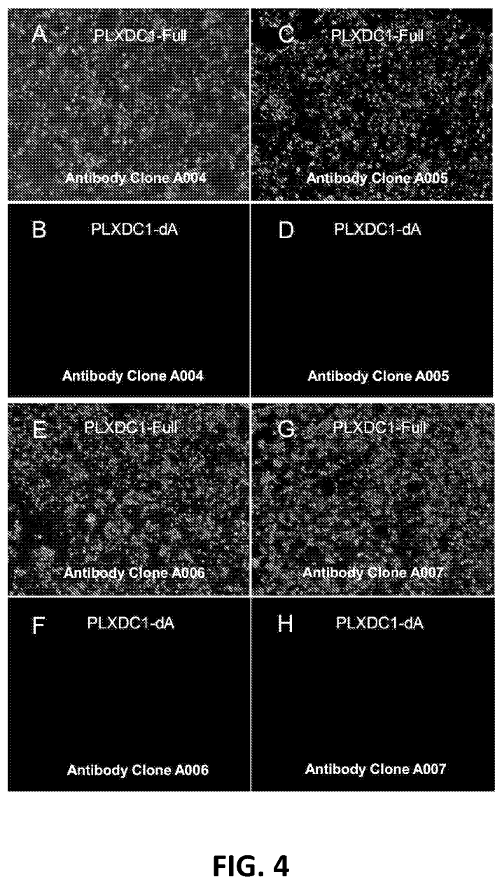

Seven of these antibodies, A001-A007, were tested for the binding specificity to Domain A. As shown in FIG. 3-4, these antibodies bound to the full PLXDC1 protein (PLXDC1-Full) but failed to bind to mutant with Domain A deleted (PLXDC1-dA).

Full length PLXDC1 (PLXDC1-Full) and domain A-deleted PLXDC1 (PLXDC1-dA) were transfected into a cell line that does not express PLXDC1. Both full length PLXDC1 and Domain A deleted PLXDC1 (PLXDC1-dA) have an epitope tag on the N-terminus. Staining using antibody against this epitope tag demonstrates that both full length PLXDC1 and Domain A-deleted PLXDC1 were well expressed (FIG. 3A-B). However, antibodies A001, A002 and A003 only bound to full length PLXDC1 but did not bind to Domain A-deleted PLXDC1 (FIG. 3C-H).

Likewise, antibodies A004, A005, A006 and A007 only bound to full length PLXDC1, but did not bind to Domain A-deleted PLXDC1 (FIG. 4A-H). This experiment indicates that these antibodies bind to PLXDC1 through Domain A.

The ability of the agonist small molecule (compound 369) in facilitating antibody binding to Domain A is demonstrated with two of the selected antibodies, A006 and A009. As shown in FIG. 5, these activating antibodies bound to standalone PLXDC1 at low nM affinity (lower curve in each panel. When compound 369 was added, however, both antibodies exhibited greatly higher affinity (upper curve in each panel).

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A method for inhibiting the growth of or killing cells in a pathogenic blood vessel in a patient in need thereof, comprising administering to the patient an antibody or antigen-binding fragment thereof that binds Domain A of a plexin domain-containing (PLXDC) protein.

2. The method of claim 1, wherein the antibody or antigen-binding fragment thereof inhibits dimerization of the Domain A.

3. The method of claim 2, wherein the antibody or antigen-binding fragment thereof binds to at least an amino acid residue involved in Domain A dimerization.

4. The method of any preceding claim, wherein the antibody or antigen-binding fragment thereof does not bind any one of Domains B-E of the PLXDC protein.

5. The method of any preceding claim, wherein the antibody or antigen-binding fragment thereof activates PLXDC signaling upon binding to the PLXDC protein.

6. The method of any preceding claim, wherein the antibody or antigen binding fragment thereof binds to the PLXDC protein with a higher affinity in the presence of a small molecule compound that binds and activates the PLXDC protein, as compared to when the small molecule compound is not present.

7. The method of any preceding claim, wherein the antibody or antigen binding fragment thereof is not capable of mediating antibody-dependent cell-mediated cytotoxicity (ADCC).

8. The method of any preceding claim, wherein the antibody is a bispecific antibody that further has a second specificity to an immune cell.

9. The method of any preceding claim, wherein the PLXDC protein is PLXDC1 or PLXDC2.

10. The method of any preceding claim, wherein the antibody or antigen binding fragment thereof is an antibody selected from Table 4 or an antigen binding fragment thereof, is an antibody or antigen binding fragment thereof that includes the complementarity-determining regions (CDR) VH CDR1, VH CDR2, VH CDR3, VL CDR1, VL CDR2 and VL CDR3 of an antibody selected from Table 4, or is an antibody or antigen binding fragment thereof that competes with an antibody selected from Table 4 in binding to PLXDC1.

11. The method of any preceding claim, wherein the antibody or antigen binding fragment thereof inhibits dimerization of the Domain A and does not include all of the CDRs of any one of antibodies A001 to A010.

12. The method of any preceding claim, wherein the patient has a disorder selected from the group consisting of diabetic retinopathy, age-related macular degeneration (AMD), retinopathy of prematurity, cancer and combinations thereof.

13. A recombinant antibody or antigen-binding fragment thereof that binds Domain A of a plexin domain-containing (PLXDC) protein.

14. The recombinant antibody or antigen-binding fragment thereof of claim 13, which inhibits dimerization of the Domain A.

15. The recombinant antibody or antigen-binding fragment thereof of claim 14, which binds to at least an amino acid residue involved in Domain A dimerization.

16. The recombinant antibody or antigen-binding fragment thereof of any one of claims 13-15, which does not bind any one of Domains B-E of the PLXDC protein.

17. The recombinant antibody or antigen-binding fragment thereof of any one of claims 13-16, which activates PLXDC signaling upon binding to the PLXDC protein.

18. The recombinant antibody or antigen-binding fragment thereof of any one of claims 13-17, which binds to the PLXDC protein with a higher affinity in the presence of a small molecule compound that binds and activates the PLXDC protein, as compared to when the small molecule compound is not present.

19. The recombinant antibody or antigen-binding fragment thereof of any one of claims 13-18, which is not capable of mediating antibody-dependent cell-mediated cytotoxicity (ADCC).

20. The recombinant antibody or antigen-binding fragment thereof of any one of claims 13-19, wherein the antibody is a bispecific antibody that further has a second specificity to an immune cell.

21. The recombinant antibody or antigen-binding fragment thereof of any one of claims 13-20, wherein the PLXDC protein is PLXDC1 or PLXDC2.

22. The recombinant antibody or antigen-binding fragment thereof of any one of claims 13-21, which is an antibody selected from Table 4 or an antigen binding fragment thereof, is an antibody or antigen binding fragment thereof that includes the complementarity-determining regions (CDR) VH CDR1, VH CDR2, VH CDR3, VL CDR1, VL CDR2 and VL CDR3 of an antibody selected from Table 4, or is an antibody or antigen binding fragment thereof that competes with an antibody selected from Table 4 in binding to PLXDC1.

23. The recombinant antibody or antigen-binding fragment thereof of any one of claims 13-22, which inhibits dimerization of the Domain A and does not include all of the CDRs of any one of antibodies A001 to A010.
